(19) United States
(12) Patent Application Publication
(10) Pub. No.: US 2024/0158503 A1
(43) Pub. Date: May 16, 2024

(54) ANTI-VSIG4 ANTIBODY OR ANTIGEN BINDING FRAGMENT AND USES THEREOF

(71) Applicants: PIERRE FABRE MEDICAMENT, LAVAUR (FR); Y-BIOLOGICS INC., DAEJEON (KR)

(72) Inventors: Noureddine LOUKILI, ORNEX (FR); Florence BAYCHELIER-TINE, Saint-Julien-en-Genevois (FR); Pierre FERRE, TOULOUSE (FR); Bum-Chan PARK, DAEJEON (KR); Jae Eun PARK, DAEJEON (KR); Hyun Mi LEE, DAEJEON (KR); Soo Young KIM, DAEJEON (KR); Hyun Ju LEE, DAEJEON (KR)

(73) Assignees: PIERRE FABRE MEDICAMENT, LAVAUR (FR); Y-BIOLOGICS INC., DAEJEON (KR)

(21) Appl. No.: 18/280,068

(22) PCT Filed: Mar. 3, 2022
(86) PCT No.: PCT/EP2022/055456

§ 371 Date: Sep. 1, 2023

(30) Foreign Application Priority Data
Mar. 3, 2021 (EP) 21305253.3

(51) Int. Cl. C07K 16/28; A61K 39/395; A61K 47/68; A61P 35/00; G01N 33/532; G01N 33/574

(52) U.S. Cl. CPC C07K 16/2827; A61K 39/3955; A61K 47/6849; A61P 35/00; G01N 33/532; G01N 33/57492; C07K 2317/21; C07K 2317/24; C07K 2317/622; G01N 2333/70532

(57) ABSTRACT

New anti-VSIG4 (V-set Ig domain-containing 4) antibodies or an antigen-binding fragments are disclosed. Uses of these antibodies, including methods of treatment, are also provided.

### TECHNICAL FIELD

The present invention relates to anti-VSIG4 (V-set Ig domain-containing 4) antibodies or an antigen-binding fragments and uses thereof.

### BACKGROUND

Macrophages are phagocytes and antigen presenting cells that differentiate from monocytes in circulating peripheral blood. They have an important role in our immune system by activating T lymphocytes, with particular relevance to cancer biology. Macrophages are important tumour-infiltrating cells and play pivotal roles in tumour growth and metastasis. In particular, tumour-associated macrophages (TAMs) can directly support tumour growth and suppress the tumour immune responses. TAMs contribute to creating an immunosuppressive tumour microenvironment through multiple routes, including triggering of inhibitory immune checkpoints in T cells. TAMs have been primarily described as having an M2-like phenotype and favour tumour growth, angiogenesis, and metastasis. A high density of TAMs with a M2 phenotype in the TME correlates with a poor survival in cancer. Switching TAMs to a predominantly M1 phenotype has thus been proposed as a key anti-cancer immunotherapeutic treatment strategy (Mills et al. (2016) Cancer Res 76: 513-516; Mantovani et al. (2017) Nat Rev Clin Oncol. 14(7): 399-416; Belgiovine et al. (2020) J Clin Med. 9(10):3226, Zhang et al. (2020) Pharmacol Res. 161: 105111; Pan et al. (2020) Front Immunol. 11: 583084; Zhou et al. (2020) Front Oncol. 10:188; Xiang et al. (2021) Signal Transduct Target Ther. 6(1): 75; Reis-Sobreiro et al. (2021) Cells. 10(9): 2364; Hourani et al. (2021) Front Oncol. 11: 788365; Jiang & Li (2022) Front Immunol. 13: 835932).

Immune checkpoint inhibitors as third-generation anti-cancer immunotherapeutic agents were first approved in 2010 by the Food and Drug Administration, and, starting from the clinical treatment for melanoma, a stream of research results showing remarkable therapeutic effects in anti-cancer therapy for lung cancer, liver cancer, or the like has continuously been published ever since. In the most recent 10 years, immune checkpoint inhibitors have become an important topic all over the world. As the anti-cancer immunotherapeutic agent is an antibody which is produced such that cancer cells are attacked by T cells, research results demonstrating that a remarkable effect is exhibited even in combination therapy with conventional anti-cancer agents are reported. As of today, various immune checkpoint proteins are known including CTLA-4 (cytotoxic T-lymphocyte-associated protein 4), PD-1 (programmed cell death protein 1), PD-L1 (programmed death-ligand 1), PD-L2 (programmed death-ligand 2), TIM-3 (T cell immunoglobulin and mucin-domain containing-3), LAG-3 (lymphocyte activation gene 3), TIGIT (T cell immunoreceptor with immunoglobulin and immunoreceptor tyrosine-based inhibitory motif domains), PSG-L1 (P-selectin glycoprotein-ligand 1), and VISTA (v-domain Ig-containing suppressor of T cell activation).

V-set Ig domain-containing 4 (VSIG4, CRIg or Z39Ig) is a B7 family-related protein which includes PDL1, VISTA and CTLA4 ligand. VSIG4 is highly expressed in tissue-resident macrophage and tumour-associated macrophage (TAM) (Lee et al. (2006) J Leukoc Biol. 80(4): 922-928; Xia et al. (2020) JCI Insight. 5(18): e141115). VSIG4 and B7 family protein share a conserved amino acid sequence. In humans, there are two different forms of the VSIG4 protein. The long form contains both a constant (C2-type) and a variable (V-type) immunoglobulin domain, whilst the short form only comprises the V-type immunoglobulin domain, with no C2-type (Vogt L. et al. (2006) J Clin Invest. 116: 2817-2826; Helmy K Y. et al. (2006) Cell 124: 915-927).

Notably, induction of VSIG4 expression is associated with several inflammatory diseases and cancers in both mice and humans (see e.g., He et al. (2008) Mol. Immunol. 45(16): 4041-4047; Xu T. et al. (2015) Am J Transl Res. 7: 1172-1180; Byun et al. (2017) Int J Gynecol Cancer. 27(5): 872-878; Zhu et al. (2018) Cancer Manag Res. 10: 3697-3705; Hu et al. (2019) Biomed Res Int. 2019: 2506843; Kim et al. (2020) J Immunol 204(Suppl. 1) 243.4). VSIG4 functions as an immune checkpoint regulator, suppressing T-lymphocyte function and promoting cancer development and progression (Zhang et al. (2016) Oncol Rep. 36(5): 2967-2975; Bianchi-Frias et al. (2019) Mol Cancer Res. 17(1): 321-331). Notably, it was reported that VSIG4 expression on macrophages is associated with the regulation of anti-tumour immunity such as development of lung cancer (Liao Y. et al. (2014) Lab Invest. 94: 706-715). In addition, VSIG4 is known to inhibit the alternative complement pathway of complement activity by binding to the subunit C3b of a convertase, thereby participating in pathogen clearance.

Antibodies directed against VSIG4 have been previously described (see e.g., WO 2020/069507). However, these antibodies only bind one of the two forms of the protein, thereby mediating only partial inhibition of its activity. Other antibodies (WO 2019/005817; Wen et al. (2017) Immunobiology. 222(6): 807-813; WO 2008/137338) block interaction between VSIG4 and C3b and are thus useful in fighting infection. However, there is no suggestion that these antibodies could be used in cancer treatment.

Thus, there is still a need to provide new anti-VSIG4 antibodies which can establish optimal anti-tumour immunity.

### DESCRIPTION OF THE INVENTION

### Objective

The object of the present disclosure is to provide a novel antibody for VSIG4, or an antigen-binding fragment thereof.

An additional object of the present disclosure is thus to provide a composition for cancer treatment comprising the aforementioned antibodies or antigen-binding fragments.

### Technical Methods to Achieve the Above Object

To achieve the above object, the present disclosure provides a monoclonal antibody specifically binding to VSIG4, or an antigen-binding fragment thereof. The antibody disclosed herein binds both the long and the short forms of VSIG4, leading to efficient suppression of VSIG4-mediated anti-inflammatory signals and reversion of the VSIG4-mediated inhibition of T-cell activation. In one aspect, the antibody disclosed herein induces repolarisation of TAM to tumour suppressive M1 macrophages, leading to T cell proliferation and tumour suppression. The anti-VSIG4 antibody disclosed herein thus activates an immune response in a patient in need thereof, thereby conferring protective anti-tumour immunity to the patient.

The present disclosure provides in particular an anti-VSIG4 monoclonal antibody, or an antigen-biding fragment thereof, having three heavy-chain CDRs and three light-chain CDRs, wherein the sequences of the CDRs are selected in the group of sequences set forth in SEQ ID NOs: 3-14. More specifically, the antibody disclosed herein comprises three heavy-chain CDRs and three light-chain CDRs as set forth in Table 2.

The present disclosure further provides an anti-VSIG4 monoclonal antibody, or an antigen-biding fragment thereof, comprising any one heavy chain variable region selected from the group consisting of the amino acid sequences of SEQ ID NOs: 45 and 47; and any one light chain variable region selected from the group consisting of the amino acid sequences of SEQ ID NOs: 46 and 48, and an antigen-binding fragment of the monoclonal antibody.

In addition, the present disclosure further provides a polynucleotide encoding the heavy chain variable region and light chain variable region of the monoclonal antibody or an antigen-binding fragment thereof.

In addition, the present disclosure further provides an expression vector comprising the polynucleotide.

In addition, the present disclosure further provides a host cell transformed with the expression vector.

In addition, the present disclosure further provides a method for producing a monoclonal antibody specifically binding to VSIG4 or an antigen-binding fragment thereof by culturing the transformant.

In addition, the present disclosure further provides a composition for stimulating an immune response comprising as an effective ingredient a monoclonal antibody specifically binding to VSIG4, or an antigen-binding fragment thereof.

In addition, the present disclosure further provides a pharmaceutical composition for treating cancer comprising as an effective ingredient a monoclonal antibody specifically binding to VSIG4, or an antigen-binding fragment thereof.

In addition, the present disclosure further provides a method for treating cancer including administering the pharmaceutical composition for treating cancer to an individual.

In addition, the present disclosure further provides an antibody-drug conjugate having a drug linked to the monoclonal antibody specifically binding to VSIG4 or an antigen-binding fragment thereof.

In addition, present disclosure further provides a CAR (chimeric antigen receptor) protein including i) above antibodies; ii) a transmembrane domain, and; iii) CAR (chimeric antigen receptor) with an intracellular signalling domain characterised by causing T cell activation according to binding of above i) antibody to an antigen.

In addition, the present disclosure still further provides a multi-specific antibody comprising with a monoclonal antibody specifically binding to VSIG4 or an antigen-binding fragment thereof.

### Benefit of the Invention

As the novel antibody of the present invention binding to VSIG4, and an antigen-binding fragment thereof can bind to VSIG4 to inhibit the activity of VSIG4, it is expected that they can be advantageously used for the development of various immunotherapeutic agents for a disorder relating to VSIG4.

### BRIEF DESCRIPTION OF THE DRAWINGS

FIG. 1 shows the structure and the expression of hVSIG4(S) and hVSIG4(L). (A) diagram illustrating the structure of the two forms of the VSIG4 protein (after Small et al., Swiss Med Wkly. (2016) 146:w14301). (B) Result of western blot for testing the expression of hVSIG4(L) and hVSIG4(S) in macrophages: rechVSIG4: recombinant hVSIG4 (long and short); 264, 265 and 266: donors from whom PBMC were isolated. AF4646: polyclonal anti-VSIG4 antibody (R&D Systems, Minneapolis, MN, USA).

FIG. 2 shows that activation of CD4⁺ T cells is inhibited by hVSIG4(S) and hVSIG4(L). CD4⁺ cells were contacted with anti CD3 OKT3 antibody (BioxCell ref BE0001-2 clone OKT3) in the presence of recombinant proteins (hVSIG4(L)-Fc, hVGIG4(S)-Fc, PDL1-Fc (R&D Systems 156-B7) or an isotype control hIgG1 (c9G4)). CD4⁺ T cells proliferation (A) and IFNγ release (B) were determined by flow cytometry.

FIG. 3 is a diagram illustrating the method disclosed herein for screening monoclonal antibodies specifically binding to VSIG4.

FIG. 4 is a diagram illustrating the expression vector for VSIG4 antigen protein.

FIG. 5 shows the result of SDS-PAGE of purified VSIG4 antigen protein.

FIG. 6 shows the result of polyphage ELISA for testing the specificity of positive poly scFv-phage antibody pool, which has been obtained through the panning process of each round (i.e., first, second, and third round), for an antigen.

FIG. 7 shows the result of ELISA for selecting positive phages with excellent binding affinity for antigen VSIG4.

FIG. 8 shows the result of SDS-PAGE analysis of recombinant human anti-VSIG4 monoclonal antibodies SA2281 and SA2297.

FIG. 9 shows the result of FACS analysis of transformed cells overexpressing human VSIG4 by using anti-human VSIG4 antibody linked with an APC fluorescent material.

FIG. 10 shows the result of FACS analysis of the binding specificity of cells overexpressing human VSIG4 for human VSIG4 antibodies SA2281 (left panel) and SA2297 (right panel). (A) HEK293E: Non-specific binding test. (B) hVSIG4/HEK293E: Specific binding to cell surfaced VSIG4.

FIG. 11 shows the binding of the human monoclonal anti-VSIG4 antibodies SA2281 and SA2297 to hVSIG4(S) and hVSIG4(L). (A) Binding to hVSIG4(S) and hVSIG4(L) was assayed by ELISA with the original scFv versions of the human anti-VSIG4 antibodies SA2281 and SA2297. (B) Binding to hVSIG4(S) and hVSIG4(L) was assayed by western blotting with the full-length human anti-VSIG4 antibodies SA2281 and SA2297. NRH: Non-reduced, heated; RH: Reduced, heated.

FIG. 12 shows that murine m6H8 and its humanised version hz6H8-A2 bind to hVSIG4(L) but not hVSIG4(S). (A) Western blot: rechVSIG4: recombinant hVSIG4 (long and short); 264, 265 and 266: donors from whom PBMS were isolated. AF4646: polyclonal anti-VSIG4 antibody (R&D Systems, Minneapolis, MN, USA). (B) ELISA with hVSIG4-His (short form) and hVSIG4 Fc (long form): m9G4: isotype control, goat IgG control: negative control.

FIG. 13 shows the result of the evaluation of anti-VSIG4 antibodies effect on VSIG4 interaction with C3b (A) or iC3b (B). Experimental points are the means of the 3 independent experiments (bars: SD).

FIG. 14 is a diagram illustrating the method disclosed herein for testing the full-length human monoclonal anti-VSIG4 antibodies in an inflammatory assay.

FIG. 15 is a diagram illustrating the method disclosed herein for testing the full-length human monoclonal anti-VSIG4 antibodies in an immunosuppression assay.

FIG. 16 is a diagram illustrating the method disclosed herein for testing the full-length human monoclonal anti-VSIG4 antibodies in a tripartite coculture assay.

### DETAILED DESCRIPTION

The present invention will become more fully understood from the detailed description given herein and from the accompanying drawings, which are given by way of illustration only and do not limit the intended scope of the invention.

### Definitions

Unless specifically defined, all technical and scientific terms used herein have the same meaning as commonly understood by a skilled artisan in chemistry, biochemistry, cellular biology, molecular biology, and medical sciences.

The term “about” or “approximately” refers to the normal range of error for a given value or range known to the person of skills in the art. It usually means within 20%, such as within 10%, or within 5% (or 1% or less) of a given value or range.

As used herein, “administer” or “administration” refers to the act of injecting or otherwise physically delivering a substance as it exists outside the body (e.g., an anti-VSIG4 antibody provided herein) into a patient, such as by mucosal, intradermal, intravenous, intramuscular delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof. The administration route of the composition of the present invention can be any of various routes including oral and parenteral routes as long as it allows delivery of the composition to a target tissue. Specifically, the administration can be made by a common method via oral, colorectal, topical, intravenous, intraperitoneal, intramuscular, intraarterial, percutaneous, intranasal, inhaling, intraocular, or intradermal route.

The terms “antibody” and “immunoglobulin” or “Ig” are used interchangeably herein. These terms are used herein in the broadest sense and specifically cover monoclonal antibodies (including full length monoclonal antibodies) of any isotype such as IgG, IgM, IgA, IgD, and IgE, polyclonal antibodies, multispecific antibodies, chimeric antibodies, and antibody fragments, provided that said fragments retain the desired biological function. These terms are intended to include a polypeptide product of B cells within the immunoglobulin class of polypeptides that is capable of binding to a specific molecular antigen and is composed of two identical pairs of polypeptide chains inter-connected by disulfide bonds, wherein each pair has one heavy chain (about 50-70 kDa) and one light chain (about 25 kDa) and each amino-terminal portion of each chain includes a variable region of about 100 to about 130 or more amino acids and each carboxy-terminal portion of each chain includes a constant region (See, Borrebaeck (ed.) (1995) Antibody Engineering, Second Ed., Oxford University Press.; Kuby (1997) Immunology, Third Ed., W.H. Freeman and Company, New York). Each variable region of each heavy and light chain is composed of three complementarity-determining regions (CDRs), which are also known as hypervariable regions and four frameworks (FRs), the more highly conserved portions of variable domains, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. In some embodiments, the specific molecular antigen can be bound by an antibody provided herein includes the target VSIG4 polypeptide, fragment or epitope. An antibody reactive with a specific antigen can be generated by recombinant methods such as selection of libraries of recombinant antibodies in phage or similar vectors, or by immunising an animal with the antigen or an antigen-encoding nucleic acid.

Antibodies also include, but are not limited to, synthetic antibodies, monoclonal antibodies, recombinantly produced antibodies, multispecific antibodies (including bi-specific antibodies), human antibodies, humanised antibodies, camelised antibodies, chimeric antibodies, intrabodies, anti-idiotypic (anti-Id) antibodies, and functional fragments of any of the above, which refers a portion of an antibody heavy or light chain polypeptide that retains some or all of the biological function of the antibody from which the fragment was derived. The antibodies provided herein can be of any type (e.g., IgG, IgE, IgM, IgD, IgA and IgY), any class (e.g., IgG1, IgG2, IgG3, IgG4, IgA1 and IgA2), or any subclass (e.g., IgG2a and IgG2b) of immunoglobulin molecule.

The terms “anti-VSIG4 antibodies” “antibodies that bind to VSIG4,” “antibodies that bind to a VSIG4 epitope,” and analogous terms are used interchangeably herein and refer to antibodies that bind to a VSIG4 polypeptide, such as a VSIG4 antigen or epitope. Such antibodies include polyclonal and monoclonal antibodies, including chimeric, humanised, and human antibodies. An antibody that binds to a VSIG4 antigen may be cross-reactive with related antigens. In some embodiments, an antibody that binds to VSIG4 does not cross-react with other antigens such as e.g., other peptides or polypeptides belonging to the B7 superfamily. An antibody that binds to VSIG4 can be identified, for example, by immunoassays, BIAcore, or other techniques known to those of skill in the art. An antibody binds to VSIG4, for example, when it binds to VSIG4 with higher affinity than to any cross-reactive antigen as determined using experimental techniques, such as radioimmunoassays (RIA) and enzyme-linked immunosorbent assays (ELISAs), for example, an antibody that specifically binds to VSIG4. Typically, a specific or selective reaction will be at least twice background signal or noise and may be more than 10 times background. See, e.g., Paul, ed., 1989, Fundamental Immunology Second Edition, Raven Press, New York at pages 332-336 for a discussion regarding antibody specificity. In some embodiments, an antibody “which binds” an antigen of interest is one that binds the antigen with sufficient affinity such that the antibody is useful as a diagnostic and/or therapeutic agent in targeting a cell or tissue expressing the antigen, and does not significantly cross-react with other proteins. In such embodiments, the extent of binding of the antibody to a “non-target” protein will be less than about 10% of the binding of the antibody to its particular target protein as determined by fluorescence activated cell sorting (FACS) analysis or radioimmunoprecipitation (RIPA). With regard to the binding of an antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-Labelled target. In this case, specific binding is indicated if the binding of the Labelled target to a probe is competitively inhibited by excess unlabelled target. The term “specific binding” or “specifically binds to” or is “specific for” a particular polypeptide or an epitope on a particular polypeptide target as used herein can be exhibited, for example, by a molecule having a K_{D} for the target of at least about 10⁻⁴ M, alternatively at least about 10⁻⁵ M, alternatively at least about 10⁻⁶ M, alternatively at least about 10⁻⁷ M, alternatively at least about 10⁻⁸ M, alternatively at least about 10⁻¹ M, alternatively at least about 10⁻¹⁰ M, alternatively at least about 10⁻¹¹ M, alternatively at least about 10⁻¹² M, or greater. In some embodiments, the term “specific binding” refers to binding where a molecule binds to a particular polypeptide or epitope on a particular polypeptide without substantially binding to any other polypeptide or polypeptide epitope. In some embodiments, an antibody that binds to VSIG4 has a dissociation constant (K_{D}) of ≤1 μM, ≤100 nM, ≤10 nM, ≤1 nM, or ≤0.1 nM.

As used herein, the term “antigen” refers to a predetermined antigen to which an antibody can selectively bind. The target antigen may be a polypeptide, carbohydrate, nucleic acid, lipid, hapten or other naturally occurring or synthetic compound. In some embodiments, the target antigen is a polypeptide, including, for example, a VSIG4 polypeptide.

The term “antigen binding fragment,” “antigen binding domain,” “antigen binding region,” and similar terms refer to that portion of an antibody which comprises the amino acid residues that interact with an antigen and confer on the binding agent its specificity and affinity for the antigen (e.g., the complementarity determining regions (CDRs)). By the expression “antigen-binding fragment” of an antibody, it is intended to indicate any peptide, polypeptide, or protein retaining the ability to bind to the target (also generally referred to as antigen) of the said antibody, generally the same epitope, and comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, or at least 200 contiguous amino acid residues, of the amino acid sequence of the antibody. In a particular embodiment, the said antigen-binding fragment comprises at least one CDR of the antibody from which it is derived. Still in a preferred embodiment, the said antigen binding fragment comprises 2, 3, 4 or 5 CDRs, more preferably the 6 CDRs of the antibody from which it is derived.

The “antigen-binding fragments” can be selected, without Limitation, in the group consisting of Fab, Fab′, (Fab′)₂, Fv, scFv (sc for single chain), Bis-scFv, scFv-Fc fragments, Fab2, Fab3, minibodies, diabodies, triabodies, tetrabodies, and nanobodies, and fusion proteins with disordered peptides such as XTEN (extended recombinant polypeptide) or PAS motifs, and any fragment of which the half-life time would be increased by chemical modification, such as the addition of poly(alkylene) glycol such as poly(ethylene) glycol (“PEGylation”) (pegylated fragments called Fv-PEG, scFv-PEG, Fab-PEG, F(ab′)₂-PEG or Fab′-PEG) (“PEG” for Poly(Ethylene) Glycol), or by incorporation in a liposome, said fragments having at least one of the characteristic CDRs of the antibody according to the invention. Among the antibody fragments, Fab has a structure including variable regions of light chain and heavy chain, a constant region of a light chain, and the first constant region of a heavy chain (CH1), and it has one antigen binding site. Fab′ is different from Fab in that it has a hinge region including one or more cysteine residues at C terminus of heavy chain CH1 domain. F(ab′)2 antibody is generated as the cysteine residues of the hinge region of Fab′ form a disulfide bond. Fv is a minimum antibody fragment which has only a heavy chain variable region and a light chain variable region, and a recombination technique for producing the Fv fragment is described in International Publication WO 88/10649 or the like. In double chain Fv (dsFv), the heavy chain variable region and light chain variable region are linked to each other via a disulfide bond, and, in single chain Fv (scFv), the heavy chain variable region and light chain variable region are covalently linked to each other via a peptide linker in general. Those antibody fragments can be obtained by using a proteinase (e.g., Fab can be obtained by restriction digestion of whole antibody with papain, and F(ab′)2 fragment can be obtained by restriction digestion with pepsin), and it can be preferably produced by genetic engineering techniques. Preferably, said “antigen-binding fragments” will be constituted or will comprise a partial sequence of the heavy or light variable chain of the antibody from which they are derived, said partial sequence being sufficient to retain the same specificity of binding as the antibody from which it is descended and a sufficient affinity, preferably at least equal to 1/100, in a more preferred manner to at least 1/10, of the affinity of the antibody from which it is descended, with respect to the target. Such antibody fragments can be found described in, for example, Harlow and Lane, Antibodies: A Laboratory Manual, Cold Spring Harbor Laboratory, New York (1989); Myers (ed.), Molec. Biology and Biotechnology: A Comprehensive Desk Reference, New York: VCH Publisher, Inc.; Huston et al., Cell Biophysics, 22:189-224 (1993); Plückthun and Skerra, Meth. Enzymol., 178:497-515 (1989) and in Day, E. D., Advanced Immunochemistry, Second Ed., Wiley-Liss, Inc., New York, NY (1990).

The terms “binds” or “binding” as used herein refer to an interaction between molecules to form a complex which, under physiologic conditions, is relatively stable. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions or forces. The strength of the total non-covalent interactions between a single antigen-binding site on an antibody and a single epitope of a target molecule, such as VSIG4, is the affinity of the antibody or functional fragment for that epitope. The ratio of association (k₁) to dissociation (k₋₁) of an antibody to a monovalent antigen (k₁/k₋₁) is the association constant K, which is a measure of affinity. The value of K varies for different complexes of antibody and antigen and depends on both k₁ and k₋₁. The association constant K for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent VSIG4, come in contact with antibodies containing multiple binding sites, the interaction of antibody with antigen at one site will increase the probability of a reaction at a second site. The strength of such multiple interactions between a multivalent antibody and antigen is called the avidity. The avidity of an antibody can be a better measure of its binding capacity than is the affinity of its individual binding sites. For example, high avidity can compensate for low affinity as is sometimes found for pentameric IgM antibodies, which can have a lower affinity than IgG, but the high avidity of IgM, resulting from its multivalence, enables it to bind antigen effectively. Methods for determining whether two molecules bind are well known in the art and include, for example, equilibrium dialysis, surface plasmon resonance, and the like. In a particular embodiment, said antibody, or antigen-binding fragment thereof, binds to VSIG4 with an affinity that is at least two-fold greater than its affinity for binding to a non-specific molecule such as BSA or casein. In a more particular embodiment, said antibody, or antigen-binding fragment thereof, binds only to VSIG4.

As used herein, the term “biological sample” or “sample” refers to a sample that has been obtained from a biological source, such as a patient or subject. A “biological sample” as used herein refers notably to a whole organism or a subset of its tissues, cells or component parts (e.g. blood vessel, including artery, vein and capillary, body fluids, including but not limited to blood, serum, mucus, lymphatic fluid, synovial fluid, cerebrospinal fluid, saliva, amniotic fluid, amniotic cord blood, urine, vaginal fluid and semen). “Biological sample” further refers to a homogenate, lysate or extract prepared from a whole organism or a subset of its tissues, cells or component parts, or a fraction or portion thereof. Lastly, “biological sample” refers to a medium, such as a nutrient broth or gel in which an organism has been propagated, which contains cellular components, such as proteins or nucleic acid molecules.

As described herein, the term “biopanning” indicates a process of selecting, from a phage library displaying a peptide on a phage coat, only the phages which display on a surface a peptide having a property of binding to a target molecule (e.g., antibody, enzyme, and cell surface receptor). In one embodiment, biopanning as used herein comprises four steps, wherein the first step is a step of preparing a phage library, the second a capturing step, involving contacting the phage library with the target molecule, the third a washing step, involving removing the phages which are not bound to the target molecule, and the fourth an elution step, whereby the phages of interest are recovered. An example of biopanning is shown in the examples of the present disclosure.

The term “block,” or a grammatical equivalent thereof, when used in the context of an antibody refers to an antibody that prevents or stops a biological activity of the antigen to which the antibody binds. A blocking antibody includes an antibody that combines with an antigen without eliciting a reaction, but that blocks another protein from later combining or complexing with that antigen. The blocking effect of an antibody can be one which results in a measurable change in the antigen's biological activity.

The terms “cell proliferative disorder” and “proliferative disorder” refer to disorders that are associated with some degree of abnormal cell proliferation. In some embodiments, the cell proliferative disorder is a tumour or cancer. “Tumour,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumour” are not mutually exclusive as referred to herein. The terms “cancer” and “cancerous” refer to or describe the physiological condition in mammals that is typically characterised by unregulated cell growth. A “cancer” as used herein is any malignant neoplasm resulting from the undesired growth, the invasion, and under certain conditions metastasis of impaired cells in an organism. The cells giving rise to cancer are genetically impaired and have usually lost their ability to control cell division, cell migration behaviour, differentiation status and/or cell death machinery. Most cancers form a tumour but some hematopoietic cancers, such as leukaemia, do not. Thus, a “cancer” as used herein may include both benign and malignant cancers. The term “cancer” as used herein refers in particular to any cancer that can be treated by the human antibody of the present disclosure without any Limitation. Examples thereof include liver cancer, breast cancer, kidney cancer, brain tumour, biliary tract cancer, oesophageal cancer, stomach cancer, colon cancer, colorectal cancer, nasopharyngeal cancer, larynx cancer, lung cancer, ascending colon cancer, cervical cancer, thyroid cancer, leukaemia, Hodgkin disease, lymphoma, and multiple myeloma blood cancer, but are not limited thereto.

A “chemotherapeutic agent” is a chemical or biological agent (e.g., an agent, including a small molecule drug or biologic, such as an antibody or cell) useful in the treatment of cancer, regardless of mechanism of action. Chemotherapeutic agents include compounds used in targeted therapy and conventional chemotherapy. Chemotherapeutic agents include, but are not limited to, alkylating agents, anti-metabolites, anti-tumour antibiotics, mitotic inhibitors, chromatin function inhibitors, anti-angiogenesis agents, anti-ooestrogens, anti-androgens or immunomodulators.

The term “chimeric” antibody refers to an antibody in which a portion of the heavy and/or light chain is derived from a particular source or species, while the remainder of the heavy and/or light chain is derived from a different source or species. In an embodiment, a “chimeric antibody” is an antibody in which the constant region, or a portion thereof, is altered, replaced, or exchanged, so that the variable region is linked to a constant region of a different species, or belonging to another antibody class or subclass. In another embodiment, a “chimeric antibody” refers to an antibody in which the variable region, or a portion thereof, is altered, replaced, or exchanged, so that the constant region is linked to a variable region of a different species, or belonging to another antibody class or subclass.

As used herein, a “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains (Kabat et al. (1977) J. Biol. Chem. 252:6609-6616; Kabat (1978) Adv. Prot. Chem. 32:1-75). The Kabat CDRs are based on sequence variability and are the most commonly used (Kabat et al. (1991) Sequences of Proteins of Immunological Interest, 5th Ed. Public Health Service, National Institutes of Health, Bethesda, MD). Chothia refers instead to the location of the structural loops (Chothia and Lesk (1987) J Mol. Biol. 196:901-917). CDR region sequences also have been defined structurally by Chothia as those residues that are not part of the conserved β-sheet framework, and thus are able to adopt different conformations (Chothia and Lesk (1987) J. Mol. Biol. 196:901-917). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). Both terminologies are well recognised in the art. CDR region sequences have also been defined by AbM, Contact and IMGT. The AbM hypervariable regions represent a compromise between the Kabat CDRs and Chothia structural loops, and are used by Oxford Molecular's AbM antibody modelling software. The “contact” hypervariable regions are based on an analysis of the available complex crystal structures. Recently, a universal numbering system has been developed and widely adopted, ImMunoGeneTics (IMGT) Information System® (Lefranc et al. (2003) Dev. Comp. Immunol. 27(1):55-77). The IMGT universal numbering has been defined to compare the variable domains whatever the antigen receptor, the chain type, or the species [Lefranc M.-P. (1997) Immunol. Today 18: 509; Lefranc M.-P. (1999) The Immunologist 7: 132-136]. In the IMGT universal numbering, the conserved amino acids always have the same position, for instance cysteine 23 (1st-CYS), tryptophan 41 (CONSERVED-TRP), hydrophobic amino acid 89, cysteine 104 (2nd-CYS), phenylalanine or tryptophan 118 (J-PHE or J-TRP). The IMGT universal numbering provides a standardised delimitation of the framework regions (FR1-IMGT: positions 1 to 26, FR2-IMGT: 39 to 55, FR3-IMGT: 66 to 104 and FR4-IMGT: 118 to 128) and of the complementarity determining regions: CDR1-IMGT: 27 to 38, CDR2-IMGT: 56 to 65 and CDR3-IMGT: 105 to 117. As gaps represent unoccupied positions, the CDR-IMGT lengths (shown between brackets and separated by dots, e.g. [8.8.13]) become crucial information. The IMGT universal numbering is used in 2D graphical representations, designated as IMGT Colliers de Perles [Ruiz, M. and Lefranc, M.-P., Immunogenetics, 53: 857-883 (2002); Kaas, Q. and Lefranc, M.-P., Current Bioinformatics, 2: 21-30 (2007)], and in 3D structures in IMGT/3Dstructure-DB [Kaas, Q., Ruiz, M. and Lefranc, M.-P., T cell receptor and MHC structural data. Nucl. Acids. Res., 32: D208-D210 (2004)]. The positions of CDRs within a canonical antibody variable domain have been determined by comparison of numerous structures (Al-Lazikani et al., J. Mol. Biol. 273:927-948 (1997); Morea et al., Methods 20:267-279 (2000)). Because the number of residues within a hypervariable region varies in different antibodies, additional residues relative to the canonical positions are conventionally numbered with a, b, c and so forth next to the residue number in the canonical variable domain numbering scheme (Al-Lazikani et al., supra (1997)). Such nomenclature is similarly well known to those skilled in the art.

Hypervariable regions may comprise “extended hypervariable regions” as follows: 24-36 or 24-34 (L1), 46-56 or 50-56 (L2) and 89-97 or 89-96 (L3) in the VL and 26-35 or 26-35A (H1), 50-65 or 49-65 (H2) and 93-102, 94-1 02, or 95-102 (H3) in the VH. The variable domain residues are 25 numbered according to Kabat et al., supra, for each of these definitions. As used herein, the terms “HVR” and “CDR” are used interchangeably.

As used herein, a “checkpoint inhibitor” refers to a molecule, such as e.g., a small molecule, a soluble receptor, or an antibody, which targets an immune checkpoint and blocks the function of said immune checkpoint. More specifically, a “checkpoint inhibitor” as used herein is a molecule, such as e.g., a small molecule, a soluble receptor, or an antibody, that is capable of inhibiting or otherwise decreasing one or more of the biological activities of an immune checkpoint. In some embodiments, an inhibitor of an immune checkpoint protein (e.g., an antagonistic antibody provided herein) can, for example, act by inhibiting or otherwise decreasing the activation and/or cell signalling pathways of the cell expressing said immune checkpoint protein (e.g., a T cell), thereby inhibiting a biological activity of the cell relative to the biological activity in the absence of the antagonist. Example of immune checkpoint inhibitors include small molecule drugs, soluble receptors, and antibodies.

The term “constant region” or “constant domain” refers to a carboxy terminal portion of the light and heavy chain which is not directly involved in binding of the antibody to antigen but exhibits various effector function, such as interaction with the Fc receptor. The terms refer to the portion of an immunoglobulin molecule having a more conserved amino acid sequence relative to the other portion of the immunoglobulin, the variable domain, which contains the antigen binding site. The constant domain contains the CH1, CH2 and CH3 domains of the heavy chain and the CL domain of the light chain.

As described herein, a “cytotoxic agent” refers to an agent which, when administered to a subject, treats or prevents the development of cell proliferation, preferably the development of cancer in the subject's body, by inhibiting or preventing a cellular function and/or causing cell death. The cytotoxic agent that can be used in the present antibody-drug conjugate includes any agent, part thereof, or residue having cytotoxic effect or inhibitory effect on cell proliferation. Examples of such agents include (i) chemotherapeutic agent capable of functioning as a microtubulin inhibitor, a mitotic inhibitor, a topoisomerase inhibitor, or a DNA interchelator; (ii) protein toxin capable of functioning enzymatically; and (iii) radioisotopes (radioactive nuclide). The cytotoxic agent may be conjugated to an antibody, such as e.g., an anti-VSIG4 antibody, to form an immunoconjugate. Preferably, the cytotoxic agent is released from the antibody under specific conditions, e.g., under acidic conditions, thereby affecting therapeutically the target cells, e.g., by preventing the proliferation thereof or by displaying a cytotoxic effect.

The term “decreased”, as used herein, refers to the level of a biomarker, e.g., VSIG4, of a subject at least 1-fold (e.g., 1, 2, 3, 4, 5, 10, 20, 30, 40, 50, 60, 70, 80, 90, 100, 1000, 10,000-fold or more) lower than its reference value. “Decreased”, as it refers to the level of a biomarker, e.g., VSIG4, of a subject, signifies also at least 5% lower (e.g., 5%, 6%, 7%, 8%, 9%, 10%, 15%, 20%, 25%, 30%, 35%, 40%, 45%, 50%, 55%, 60%, 65%, 70%, 75%, 80%, 85%, 90%), 95%), 99%), or 100%) than the level in the reference sample or with respect to the reference value for said marker.

The term “detecting” as used herein encompasses quantitative or qualitative detection.

The term “detectable probe” or “detectable agent,” as used herein, refers to a composition that provides a detectable signal. The term refers to a substance that can be used to ascertain the existence or presence of a desired molecule, such as an antibody provided herein, in a sample or subject. A detectable agent can be a substance that is capable of being visualised or a substance that is otherwise able to be determined and/or measured (e.g., by quantitation). The term includes, without Limitation, any fluorophore, chromophore, radiolabel, enzyme, antibody or antibody fragment, and the like, that provide a detectable signal via its activity.

As used herein, “diagnosis” or “identifying a subject having” refers to a process of identifying a disease, condition, or injury from its signs and symptoms. A diagnosis is notably a process of determining if an individual is afflicted with a disease or ailment (e.g., cancer). Cancer is diagnosed for example by detecting either the presence of a marker associated with cancer such as, e.g., VSIG4.

The term “encode” or grammatical equivalents thereof as it is used in reference to nucleic acid molecule refers to a nucleic acid molecule in its native state or when manipulated by methods well known to those skilled in the art that can be transcribed to produce mRNA, which is then translated into a polypeptide and/or a fragment thereof. The antisense strand is the complement of such a nucleic acid molecule, and the encoding sequence can be deduced therefrom.

An “effective amount” or “therapeutically effective amount” of an agent, e.g., a pharmaceutical formulation, refers to an amount effective, at dosages and for periods of time necessary, to elicit the desired biological response in a subject. Such response includes alleviation of the symptoms of the disease or disorder being treated, prevention, inhibition or a delay in the recurrence of symptom of the disease or of the disease itself, an increase in the longevity of the subject compared with the absence of the treatment, or prevention, inhibition or delay in the progression of symptom of the disease or of the disease itself. An “effective amount” is in particular the amount of the agent effective to achieve the desired therapeutic or prophylactic result More specifically, an “effective amount” as used herein is an amount of the agent that confers a therapeutic benefit. A therapeutically effective amount is also one in which any toxic or detrimental effects of the agent are outweighed by the therapeutically beneficial effects.

An effective amount can be administered in one or more administrations, applications or dosages. Such delivery is dependent on a number of variables including the time period for which the individual dosage unit is to be used, the bioavailability of the agent, the route of administration, etc. In some embodiments, effective amount also refers to the amount of an antibody (e.g., an anti-VSIG4 antibody) provided herein to achieve a specified result (e.g., inhibition of an immune checkpoint biological activity, such as modulating T cell activation). In some embodiments, this term refers to the amount of a therapy (e.g., an immune checkpoint inhibitor such as e.g., an anti-VSIG4 antibody) which is sufficient to reduce and/or ameliorate the severity and/or duration of a given disease, disorder or condition and/or a symptom related thereto. This term also encompasses an amount necessary for the reduction or amelioration of the advancement or progression of a given disease, disorder or condition, reduction or amelioration of the recurrence, development or onset of a given disease, disorder or condition, and/or to improve or enhance the prophylactic or therapeutic effect(s) of another therapy (e.g., a therapy other than said immune checkpoint inhibitor). In the context of cancer therapy, a therapeutic benefit means for example any amelioration of cancer, including any one of, or combination of, halting or slowing the progression of cancer (e.g., from one stage of cancer to the next), halting or delaying aggravation or deterioration of the symptoms or signs of cancer, reducing the severity of cancer, inducing remission of cancer, inhibiting tumour cell proliferation, tumour size, or tumour number, or reducing levels of biomarker(s) indicative of the cancer. In some embodiments, the effective amount of an antibody is from about 0.1 mg/kg (mg of antibody per kg weight of the subject) to about 100 mg/kg. In some embodiments, an effective amount of an antibody provided therein is about 0.1 mg/kg, about 0.5 mg/kg, about 1 mg/kg, 3 mg/kg, 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg about 90 mg/kg or about 100 mg/kg (or a range therein).

The term “epitope” as used herein refers to the region of an antigen, such as VSIG4 polypeptide or VSIG4 polypeptide fragment, to which an antibody binds. Preferably, an epitope as used herein is a localised region on the surface of an antigen, such as VSIG4 polypeptide or VSIG4 polypeptide fragment, that is capable of being bound to one or more antigen binding regions of an antibody, and that has antigenic or immunogenic activity in an animal, such as a mammal (e.g., a human), that is capable of eliciting an immune response. An epitope having immunogenic activity is a portion of a polypeptide that elicits an antibody response in an animal. An epitope having antigenic activity is a portion of a polypeptide to which an antibody binds as determined by any method well known in the art, for example, by an immunoassay. Antigenic epitopes need not necessarily be immunogenic. Epitopes usually consist of chemically active surface groupings of molecules such as amino acids and have specific three-dimensional structural characteristics as well as specific charge characteristics. In certain embodiments, epitopes may include determinants that are chemically active surface groupings of molecules such as sugar side chains, phosphoryl groups, or sulfonyl groups, and, in certain embodiments, may have specific three-dimensional structural characteristics, and/or specific charge characteristics. An epitope can be formed by contiguous residues or by non-contiguous residues brought into close proximity by the folding of an antigenic protein. Epitopes formed by contiguous amino acids are typically retained on exposure to denaturing solvents, whereas epitopes formed by non-contiguous amino acids are typically lost under said exposure. Generally, an antigen has several or many different epitopes and reacts with many different antibodies. The determination of the epitope bound by an antibody may be performed by any epitope mapping technique known to a person skilled in the art.

The terms “full-length antibody,” “intact antibody” or “whole antibody” are used interchangeably to refer to an antibody in its substantially intact form, as opposed to an antibody fragment. Specifically, “full-length antibodies” as used herein include those with heavy and light chains including an Fc region. The constant domains may be native sequence constant domains (e.g., human native sequence constant domains) or amino acid sequence variants thereof. In some cases, the intact antibody may have one or more effector functions.

The term “glycosylation” described herein means a processing method for delivering a glycosyl group to a protein. Glycosylation is effected by binding of a glycosyl group to a serine, a threonine, an asparagine, or a hydroxylysine residue of a target protein as mediated by a glycosyl transferase. The glycosylated protein not only can be used as a constitutional material of a living tissue but also plays an important role in cell recognition on a cell surface. As such, according to the present invention, by changing the glycosylation or pattern of the glycosylation of the monoclonal antibody of the present invention or an antigen-binding fragment thereof, an enhanced effect of the antibody can be obtained.

The term “heavy chain” when used in reference to an antibody refers to a polypeptide chain of about 50-70 kDa, wherein the amino-terminal portion includes a variable region of about 120 to 130 or more amino acids and a carboxy-terminal portion that includes a constant region. The constant region can be one of five distinct types, referred to as alpha (α), delta (δ), epsilon (ε), gamma (γ) and mu (μ), based on the amino acid sequence of the heavy chain constant region. The distinct heavy chains differ in size: α, δ and γ contain approximately 450 amino acids, while p and E contain approximately 550 amino acids. When combined with a light chain, these distinct types of heavy chains give rise to five well known classes of antibodies, IgA, IgD, IgE, IgG and IgM, respectively, including four subclasses of IgG, namely IgG1, IgG2, IgG3 and IgG4. A heavy chain can be a human heavy chain.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is an antibody that possesses an amino-acid sequence corresponding to that of an antibody produced by a human and/or has been made using any of the techniques for making human antibodies as disclosed herein. This definition of a human antibody specifically excludes a humanised antibody comprising non-human antigen-binding residues. Human antibodies can be produced using various techniques known in the art, including phage-display libraries, as disclosed herein. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can also be prepared by administering the antigen to a transgenic animal that has been modified to produce such antibodies in response to antigenic challenge, but whose endogenous loci have been disabled, e.g., immunised xenomice (see, e.g., U.S. Pat. Nos. 6,075,181 and 6,150,584 regarding XENOMOUSE™ technology). See also, for example, Li et al., Proc. Nati. Acad. Sci. USA, 103:3557-3562 (2006) regarding human antibodies generated via a human B-cell hybridoma technology.

A “humanised” antibody refers to a chimeric antibody that contains minimal sequence derived from non-human immunoglobulin. In one embodiment, a humanised antibody is a human immunoglobulin (recipient antibody) in which residues from a CDR of the recipient are replaced by residues from a CDR of a non-human species (donor antibody) such as mouse, rat, rabbit, or nonhuman primate having the desired specificity, affinity, and/or capacity. In some instances, some of the skeleton segment residues (called FR for framework) can be modified to preserve binding affinity, according to techniques known by a man skilled in the art (Jones et al., Nature, 321:522-525, 1986). In some embodiments, FR residues of the human immunoglobulin are replaced by corresponding non-human residues. In certain embodiments, a humanised antibody will comprise substantially all of at least one, and typically two, variable domains, in which all or substantially all of the CDRs correspond to those of a non-human antibody, and all or substantially all of the FRs correspond to those of a human antibody. A humanised antibody optionally may comprise at least a portion of an antibody constant region (Fc), typically that of a human immunoglobulin. A “humanised form” of an antibody, e.g., a non-human antibody, refers to an antibody that has undergone humanisation. The goal of humanisation is a reduction in the immunogenicity of a xenogenic antibody, such as a murine antibody, for introduction into a human, while maintaining the full antigen binding affinity and specificity of the antibody. For further details, see, e.g., Jones et al, Nature 321: 522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & t Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409

As used herein, “identifying” as it refers to a subject that has a condition refers to the process of assessing a subject and determining that the subject has a condition, for example, suffers from cancer.

As used herein, the terms “immune checkpoint” or “immune checkpoint protein” refer to certain proteins made by some types of immune system cells, such as T cells, and some cancer cells. Such proteins regulate T cell function in the immune system. Notably, they help keep immune responses in check and can keep T cells from killing cancer cells. Said immune checkpoint proteins achieve this result by interacting with specific ligands which send a signal into the T cell and essentially switch off or inhibit T cell function. Inhibition of these proteins results in restoration of T cell function and an immune response to the cancer cells. Examples of checkpoint proteins include, but are not limited to CTLA-4, PDL1, PDL2, PD1, B7-H3, B7-H4, BTLA, HVEM, TIGIT, TIM3, GAL9, LAG3, VISTA, KIR, 2B4 (belongs to the CD2 family of molecules and is expressed on all NK, γδ, and memory CD8+ (αβ) T cells), CD160 (also referred to as BY55), CGEN-15049, CHK1 and CHK2 kinases, IDO1, A2aR, and various B7 family ligands.

The term “increased”, as used herein, refers to the level of a biomarker, e.g. VSIG4, of a subject at least 1-fold (e.g. 1, 2, 3, 4, 5, 10, 20, 30, 40, 50, 60, 70, 80, 90, 100, 1000, 10,000-fold or more) greater than its reference value. “Increased”, as it refers to the level of a biomarker, e.g., VSIG4, of a subject, signifies also at least 5% greater (e.g., 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or 100%) than the level in the reference sample or with respect to the reference value for said marker.

As used herein, an “inhibitor” or “antagonist” refers to a molecule that is capable of inhibiting or otherwise decreasing one or more of the biological activities of a target protein, such as any one of the immune checkpoint proteins described above.

An “isolated” antibody is one which has been separated from a component of its natural environment. In some embodiments, an antibody is purified to greater than 95% or 99% purity as determined by, for example, electrophoresis (e.g., SDS-PAGE, isoelectric focusing (IEF), capillary electrophoresis) or chromatography (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “K_{D}” used herein means a dissociation constant of a specific antibody-antigen interaction and is used as an indicator for measuring the affinity of an antibody for an antigen. Lower K_{D} means higher affinity of an antibody for an antigen.

As intended herein, the “level” of a biomarker, e.g., VSIG4, consists of a quantitative value of the biomarker in a sample, e.g., in a sample collected from a cancer-suffering patient. In some embodiments, the quantitative value does not consist of an absolute value that is actually measured, but rather consists of a final value resulting from taking into consideration of a signal to noise ratio occurring with the assay format used, and/or taking into consideration of calibration reference values that are used to increase reproducibility of the measures of the level of a cancer marker, from assay-to-assay. In some embodiments, the “level” of a biomarker, e.g. VSIG4, is expressed as arbitrary units, since what is important is that the same kind of arbitrary units are compared (i) from assay-to-assay, or (ii) from one cancer-suffering patient to others, or (iii) from assays performed at distinct time periods for the same patient, or (iv) between the biomarker level measured in a patient's sample and a predetermined reference value (which may also be termed a “cut-off” value herein).

The term “light chain” when used in reference to an antibody refers to a polypeptide chain of about 25 kDa, wherein the amino-terminal portion includes a variable region of about 100 to about 110 or more amino acids and a carboxy-terminal portion that includes a constant region. The approximate length of a light chain is 211 to 217 amino acids. There are two distinct types, referred to as kappa (κ) of lambda (λ) based on the amino acid sequence of the constant domains. Light chain amino acid sequences are well known in the art. A light chain can be a human light chain.

A “macrophage” as used herein is a myeloid immune cell which is part of the mononuclear phagocyte system. Macrophages are present in virtually all tissues of the body, where they differentiate into tissue-resident macrophages, thus forming a network of specialised cells, including alveolar macrophages (lung), red pulp macrophages (spleen), Kupffer cells (liver), microglia (brain), Langerhans cells (epidermis), osteo-clasts (bone), and histiocytes (connective tissue). Macrophages are phagocytes and antigen presenting cells that differentiate from monocytes in circulating peripheral blood. They also produce many important cytokines at all stages of the immune response, including the tissue repair phase. They play an important role in both innate and adaptive immunity by activating T lymphocytes.

Mature macrophages can be divided into two populations, according to their activation state and functions, i.e., M1-type (classically activated macrophage) and M2-type (alternatively activated macrophage). M1 macrophages, also referred to as “killer macrophages”, are characterised by the production of high levels of pro-inflammatory cytokines, an ability to mediate resistance to pathogens, strong microbicidal properties, high production of reactive nitrogen and oxygen intermediates, and promotion of Th1 responses. They can express one or more of CD80, CD86, CD197, HLA-DR, and CD40 on their cell membrane; advantageously they express more than one of these markers. M1 polarisation is triggered by LPS, IFN-γ and granulocyte-macrophage colony stimulating factor (GM-CSF), and results in secretion of proinflammatory cytokines such as IL-1β, TNF-α, IL-12p70, IL-6, IL-18 and IL-23.

Macrophages that activate Th2 T lymphocytes provide an anti-inflammatory response and are denoted M2 macrophages. M2 macrophage activation is induced by fungal cells, immune complexes, helminth infections, complement components, apoptotic cells, macrophage colony stimulating factor (MCSF), IL-4, IL-13, IL-10 and TGF-β. M2 macrophages play an important role in wound healing and tissue repair. In addition, they have pro-tumoural functions, including e.g., tumour invasion, metastasis, tumour cell proliferation, tumour growth, tumour survival, neo-angiogenesis, suppression of adaptive or innate immunity and extracellular matrix remodelling. M2 macrophages mainly secrete Arginase-I, IL-10 and TGF-β and other anti-inflammatory cytokines, which have the function of reducing inflammation and contributing to tumour growth and immunosuppressive function. Cell surface markers associated with the M2 phenotype include CD163, CD206, CD200R, CD209. M2 macrophages thus express one or more of these markers; advantageously they express more than one of these markers.

As used herein, the term “tumour associated macrophages” (TAMs) generally refers to macrophages that exist in the microenvironment of a cancer, for example, a tumour. Originating from both tissue resident macrophages and circulating monocytes, TAMs may be composed of multiple distinct populations with overlapping features that depend on a variety of factors including location in the microenvironment, stage of the tumour, and type of cancer. TAM subsets may advantageously be classified as tumouricidal vs. tumour-promoting, often referred as M1/M2 macrophages, based on the expression of specific markers. M1 TAMs possess proinflammatory and antitumour activities (anti-tumourigenic) whilst M2 are mainly involved in suppressing inflammation and promoting tumour growth (pro-tumourigenic). TAMs are predominantly polarised to M2 and thus favour tumour growth, angiogenesis, and metastasis.

TAMs perform a prominent role in modulating immune responses to tumours. The mechanisms of immunosuppression employed by TAMs are targeted toward inhibiting the activity of the adaptative immune system, namely T-cells, and NK cells. TAMs do so by direct cell-cell interaction with target cells, or through secreted factors. Preferably, TAMs utilise at least one of the four following distinct functions to suppress T-cell mediated immunity: (1) signalling via immune checkpoint inhibitors such as e.g., PD-1 and CTLA-4; (2) depriving the local environment of nutrients necessary for T-cell activation and function; (3) generation of nitric oxygen and reactive nitrogen species, by iNOS expression; and (4) production of reactive oxygen species. More preferably, these effects ultimately lead to a decrease in the effect and numbers of anti-tumour T-cells whilst enhancing the population of tumour-supporting regulatory T-cells.

As used herein, the term “monoclonal antibody” designates an antibody arising from a nearly homogeneous antibody population, wherein population comprises identical antibodies except for a few possible naturally-occurring mutations which can be found in minimal proportions. A monoclonal antibody arises from the growth of a single cell clone, such as a hybridoma, and is characterised by heavy chains of one class and subclass, and light chains of one type. As used herein, a monoclonal antibody shows specific binding to a single antigenic site (i.e., single epitope) when the antibody is presented to it. The monoclonal antibody can be produced by various methods that are well known in the corresponding technical area.

As described herein, the term “PEGylation” means a processing method for increasing the retention time of an antibody in blood by introducing polyethylene glycol to the aforementioned monoclonal antibody or an antigen-binding fragment thereof. Specifically, according to PEGylation of polymer nanoparticles with polyethylene glycol, hydrophilicity on a nanoparticle surface is enhanced, and, accordingly, fast degradation in living body can be prevented due to so-called stealth effect which prevents recognition by immune activity including macrophage in a human body to cause phagocytosis and digestion of pathogens, waste products, and foreign materials introduced from an outside. As such, the retention time of an antibody in blood can be increased by PEGylation. The PEGylation employed in the present disclosure can be carried out by a method by which an amide group is formed based on a bond between the carboxyl group of hyaluronic acid and the amine group of polyethylene glycol, but it is not limited thereto, and the PEGylation can be carried out by various methods. At that time, as for the polyethylene glycol to be used, polyethylene glycol having molecular weight of 100 to 1,000 and a linear or branched structure is preferably used, although it is not particularly limited thereto.

As used herein, the “percentage identity” or “% identity” between two sequences of nucleic acids or amino acids refers to the percentage of identical nucleotides or amino acid residues between the two sequences to be compared, obtained after optimal alignment, this percentage being purely statistical and the differences between the two sequences being distributed randomly along their length. The comparison of two nucleic acid or amino acid sequences is traditionally carried out by comparing the sequences after having optimally aligned them, said comparison being able to be conducted by segment or by using an “alignment window”. Optimal alignment of the sequences for comparison can be carried out, in addition to comparison by hand, by means of methods known by a man skilled in the art.

For the amino acid sequence exhibiting at least 70%, at least 75%, at least 80%, at least 85%, at least 90%, at least 91%, at least 92%, at least 93%, at least 94%, at least 95%, at least 96%, at least 97%, at least 98%, or at least 99% identity with a reference amino acid sequence, preferred examples include those containing the reference sequence, certain modifications, notably a deletion, addition or substitution of at least one amino acid, truncation or extension. In the case of substitution of one or more consecutive or non-consecutive amino acids, substitutions are preferred in which the substituted amino acids are replaced by “equivalent” amino acids. Here, the expression “equivalent amino acids” is meant to indicate any amino acids likely to be substituted for one of the structural amino acids without however modifying the biological activities of the corresponding antibodies and of those specific examples defined below. Equivalent amino acids can be determined either on their structural homology with the amino acids for which they are substituted or on the results of comparative tests of biological activity between the various antibodies likely to be generated.

As a non-limiting example, Table 1 below summarises the possible substitutions likely to be carried out without resulting in a significant modification of the biological activity of the corresponding modified antigen binding protein; inverse substitutions are naturally possible under the same conditions.

TABLE 1

| Original residue | Substitution(s) |
|---|---|
| Ala (A) | Val, Gly, Pro |
| Arg (R) | Lys, His |
| Asn (N) | Gln |
| Asp (D) | Glu |
| Cys (C) | Ser |
| Gln (Q) | Asn |
| Glu (E) | Asp |
| Gly (G) | Ala |
| His (H) | Arg |
| Ile (I) | Leu |
| Leu (L) | Ile, Val, Met |
| Lys (K) | Arg |
| Met (M) | Leu |
| Phe (F) | Tyr |
| Pro (P) | Ala |
| Ser (S) | Thr, Cys |
| Thr (T) | Ser |
| Trp (W) | Tyr |
| Tyr (Y) | Phe, Trp |
| Val (V) | Leu, Ala |

The term “pharmaceutically acceptable” as used herein means being approved by a regulatory agency of the Federal or a state government, or listed in the U.S. Pharmacopeia, European Pharmacopeia or other generally recognised Pharmacopeia for use in animals, and more particularly in humans. More specifically, when referring to a carrier, the expression “pharmaceutically acceptable” means that the carrier(s) is compatible with the other ingredient(s) of the composition and is not deleterious to the recipient thereof. Accordingly, as used herein, the expression “pharmaceutically acceptable carrier” refers to a carrier or a diluent which does not inhibit the biological activity and characteristics of a compound for administration without stimulating a living organism. The type of carrier can be selected based upon the intended route of administration. The amount of each carriers used may vary within ranges conventional in the art. As a pharmaceutically acceptable carrier in the composition which is prepared as a liquid solution, physiological saline, sterilised water, buffered saline, albumin injection solution, dextrose solution, maltodextrin solution, glycerol, and a mixture of one or more of them can be used as a sterilised carrier suitable for a living organism. If necessary, common additives like anti-oxidant, buffer solution, and bacteriostat may be added. Furthermore, by additionally adding a diluent, a dispersant, a surfactant, a binder, or a lubricant, the composition can be prepared as a formulation for injection like aqueous solution, suspension, and emulsion, a pill, a capsule, a granule, or a tablet.

As used herein, the term “polyclonal antibody” refers to an antibody which was produced among or in the presence of one or more other, non-identical antibodies. In general, polyclonal antibodies are produced from a B-lymphocyte in the presence of several other B-lymphocytes producing non-identical antibodies. Usually, polyclonal antibodies are obtained directly from an immunised animal.

The term “reference value”, as used herein, refers to the expression level of a biomarker under consideration (e.g., VSIG4) in a reference sample. A “reference sample”, as used herein, means a sample obtained from subjects, preferably two or more subjects, known to be free of the disease or, alternatively, from the general population. The suitable reference expression levels of biomarker can be determined by measuring the expression levels of said biomarker in several suitable subjects, and such reference levels can be adjusted to specific subject populations. The reference value or reference level can be an absolute value; a relative value; a value that has an upper or a lower limit; a range of values; an average value; a median value, a mean value, or a value as compared to a particular control or baseline value. A reference value can be based on an individual sample value such as, for example, a value obtained from a sample from the subject being tested, but at an earlier point in time. The reference value can be based on a large number of samples, such as from population of subjects of the chronological age matched group, or based on a pool of samples including or excluding the sample to be tested.

A “subject” which may be subjected to the methodology described herein may be any of mammalian animals including human, dog, cat, cattle, goat, pig, swine, sheep and monkey. A human subject can be known as a patient. In one embodiment, “subject” or “subject in need” refers to a mammal that is suffering from cancer or is suspected of suffering from cancer or has been diagnosed with cancer. As used herein, a “cancer-suffering subject” refers to a mammal that is suffering from cancer or has been diagnosed with cancer. A “control subject” refers to a mammal that is not suffering from cancer, and is not suspected of suffering from cancer.

As used herein, “treating” a disease in a subject or “treating” a subject having a disease refers to subjecting the subject to a pharmaceutical treatment, e.g., the administration of a drug, such that the extent of the disease is decreased or prevented. For example, treating results in the reduction of at least one sign or symptom of the disease or condition. Treatment includes (but is not limited to) administration of a composition, such as a pharmaceutical composition, and may be performed either prophylactically, or subsequent to the initiation of a pathologic event. Treatment can require administration of an agent and/or treatment more than once.

The “variable region” or “variable domain” of an antibody refers to the amino-terminal domains of the heavy or light chain of the antibody. The variable domain of the heavy chain may be referred to as “V_{H}.” The variable domain of the light chain may be referred to as “V_{L}.” These domains are generally the most variable parts of an antibody and contain the antigen-binding sites.

The term “vector” refers to a substance that is used to introduce a nucleic acid molecule into a host cell. In particular, a “vector,” as used herein, is a nucleic acid molecule capable of propagating another nucleic acid molecule to which it is linked. One example of vector is a “plasmid”, which refers to a circular double stranded DNA loop into which additional DNA segments may be ligated. Another example of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. The term “vector” thus includes the vector as a self-replicating nucleic acid structure as well as the vector incorporated into the genome of a host cell into which it has been introduced. Vectors applicable for use include, for example, expression vectors, plasmids, phage vectors, viral vectors, episomes and artificial chromosomes, which can include selection sequences or markers operable for stable integration into a host cell's chromosome.

Certain vectors are capable of directing the expression of genes to which they are operatively linked. Such vectors are referred to herein as “recombinant expression vectors” (or simply, “expression vectors”). In general, expression vectors of utility in recombinant DNA techniques are in the form of plasmids. In the present specification, “plasmid” and “vector” may be used interchangeably as the plasmid is the most commonly used form of vector. However, the invention is intended to include such forms of expression vectors, such as bacterial plasmids, YACs, cosmids, retrovirus, EBV-derived episomes, and all the other vectors that the skilled man will know to be convenient for ensuring the expression of the heavy and/or light chains of the antibody of interest (e.g., an anti-VSIG4 antibody). The skilled man will realise that the polynucleotides encoding the heavy and the light chains can be cloned into different vectors or in the same vector.

The vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more nucleic acid molecules are to be co-expressed (e.g. both an antibody heavy and light chain), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the nucleic acid molecule is expressed in a sufficient amount to produce the desired product (e.g. an anti-VSIG4 antibody provided herein), and it is further understood that expression levels can be optimised to obtain sufficient expression using methods well known in the art.

The term “VSIG4” or “VSIG4 polypeptide” and similar terms refers to the polypeptide (“polypeptide,” “peptide” and “protein” are used interchangeably herein) encoded by the human V-set and immunoglobulin domain containing 4 (VIG4) gene, which is located in the pericentromeric region of the human X chromosome, and is also known in the art as immunoglobulin superfamily protein Z391G, Z391G, complement receptor of the immunoglobulin superfamily, CRIg. VSIG4 gene sequence may be for example represented by a sequence having a GenBank accession number such as e.g., No. NM_007268.2, NM_001100431.1, NM_001184831.1, NM_001184830.1, or NM_001257403.1.

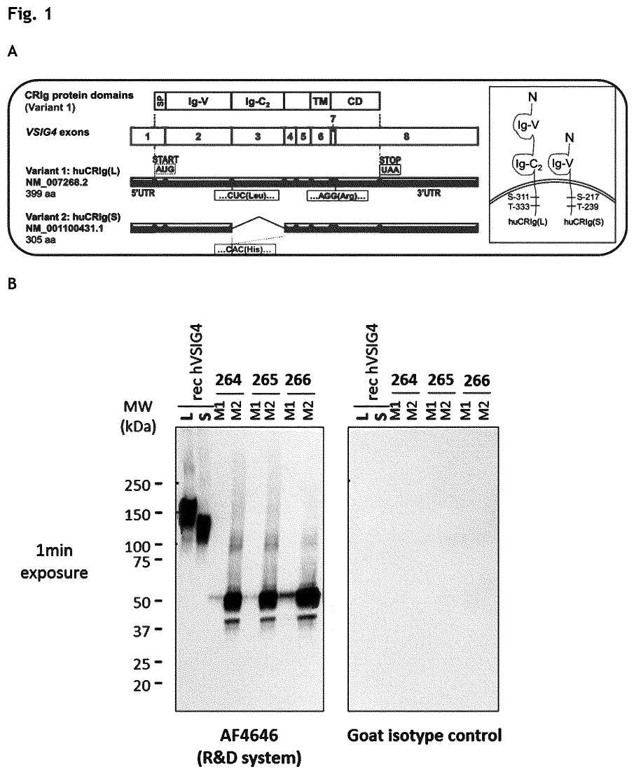

VSIG4 (V-set and Ig domain-containing 4) is a v-set and immunoglobulin-domain containing protein that is structurally related to the B7 family of immune regulatory proteins. In humans, there are two different forms of the VSIG4 protein. The long form contains both a constant (C2-type) and a variable (V-type) immunoglobulin domain, whilst the short form only comprises the V-type immunoglobulin domain, with no C2-type. These two forms are illustrated in FIG. 1A. In one embodiment, the human VSIG4 protein has a sequence represented by the sequence of Uniprot accession number Q9Y279. In one embodiment, the long form of human VSIG4 protein has a sequence represented by the sequence of Uniprot accession number Q9Y279-1. Preferably, the long form of VSIG4 has the sequence set forth in SEQ ID No. 1. In one embodiment, the short form of human VSIG4 protein has a sequence represented by the sequence of Uniprot accession number Q9Y279-3. Preferably, the short form of VSIG4 has the sequence set forth in SEQ ID No. 2.

VSIG4 functions as a complement receptor, functionally inhibiting the complement activity by binding to the complement iC3b and C3b segments thereby mediating clearance of C3b-opsonised pathogens. VSIG4 expression has been observed to be restricted to tissue macrophages, and it has been shown to be downregulated in response to lipopolysaccharide (LPS) (Vogt et al. (2006) J. of Clin. Invest. 116:2817). On the other hand, VSIG4 is highly expressed in TAMs (Lee et al. (2006) J Leukoc Biol. 80(4): 922-928; Xia et al. (2020) JCI Insight. 5(18): e141115).

VSIG4 is an immune checkpoint protein, with anti-inflammatory and immunosuppressive properties. A soluble VSIG4 fusion protein inhibits inflammation (Small et al., Swiss Med Wkly. (2016) 146: w14301), whereas VSIG4-deficiency initiates macrophage-mediated inflammation (Liao et al. (2014) Lab. Invest. 94:706). This inhibition of macrophage activation by VSIG4 appears to be C3b-independent (Li et al. (2017) Nat Commun. 8(1):1322). VSIG4 has a regulatory function in T cell activation (Vogt et al. (2006) J. of Clin. Invest. 116: 2817; Xu et al. (2010) Immunol Lett. 128(1):46-50; Jung et al. (2012) Hepatology. 56(5): 1838-48; Jung et al. (2015) Immunol Lett. 165(2):78-83; Munawara et al. (2019) Front Immunol. 10:2892). Notably, VSIG4 is a strong negative regulator of T-cell proliferation and IL-2 production by binding an unidentified T-cell ligand receptor (Vogt et al. (2006) J. of Clin. Invest. 116:2817).

As with many immune checkpoint proteins, VSIG4 activity facilitates tumour growth by promoting immune tolerance. Vsig4-deficient mice grow smaller tumours than wild-type, suggesting that the absence of VSIG4 activates an immune response which prevents tumour growth. Massive infiltrates of VSIG4-expressing macrophages into the tumour microenvironment have been observed in patients diagnosed with non-small cell lung cancer (Liao et al. (2014) Lab. Invest. 94:706). The VSIG4 gene is overexpressed on several kind of cancer cells, such as lung cancer, ovarian cancer, breast cancer, hepatoma, and multiple melanoma, and acts like an oncogene which suppresses immune responses and promote tumour progression. High VSIG4 expression has indeed been correlated with high-grade glioma and poor patient prognosis (Xu et al. (2015) Am. J. Transl. Res. 7: 1172).

### Anti-VSIG4 Antibodies

Immune checkpoints play crucial roles in maintaining self-tolerance and limiting immune-mediated tissue damage under physiologic conditions. VSIG4 is a type-I transmembrane protein belonging to the B7-related immunoglobulin superfamily which is highly expressed in tissue-resident macrophage and tumour-associated macrophage. VSIG4 is a coinhibitory ligand that negatively regulates T-cell activation through inhibiting CD4⁺ and CD8⁺ T-cell proliferation and IL-2 production. Two forms of VSIG4 are known, a long form (huVSIG4(L)) and a short form (huVSIG4(S)), which differ by the presence of a membrane proximal domain that is an IgC-type immunoglobulin domain in the long form.

The present inventors have now shown that both forms are expressed in M2 macrophages. Notably, both forms are expressed in tumours. Furthermore, both forms are functional: soluble versions of either huVSIG4(L) or huVSIG4(S) inhibit human CD4⁺ and CD8⁺ T-cell activation, as evidenced by inhibition of T-cell proliferation and IFNγ production. Both the long and the short forms of VSIG4 thus contribute to the regulatory activity of the protein, which means that both must be inhibited for immunosuppression to be relieved.

The present disclosure provides new monoclonal antibodies specifically binding to human VSIG4. More specifically, the present disclosure provides new monoclonal antibodies capable of binding to both the long form et the short form of the protein, in contrast to the antibodies of the prior art (e.g., WO 2020/069507).

The inventors have found that effective VSIG4 blockade is achieved with the anti-VSIG4 antibodies disclosed herein. Indeed, these antibodies antagonise VSIG4 interaction with either C3b or iC3b. Moreover, the antibodies disclosed herein modulate VSIG4 anti-inflammatory activity by VSIG4-expressing macrophages, as evidenced by their ability to trigger the release of pro-inflammatory cytokines whilst blocking the secretion of anti-inflammatory cytokines. The present antibodies also interfere with VSIG4-induced immune suppression of T-cell responses by VSIG4-expressing macrophages. Preliminary results obtained in the coculture model confirm the inhibition of VSIG4 anti-inflammatory and immunosuppressive functions demonstrate in vitro assays. The present anti-VSIG4 antibodies induce polarisation of macrophages, notably TAMs, to an M1 phenotype with associated pro-inflammatory cytokines release and induce T cell activation that promote the killing of cancer cells. Thus, the anti-VSIG4 antibodies disclosed herein promote the polarisation of macrophages, notably TAMs, to the M1 phenotype, thereby conferring protecting anti-tumour immunity.

The anti-VSIG4 antibodies disclosed herein are therefore useful for generating an anti-tumour immune response in cancer patients.

In a first aspect, the present disclosure provides a monoclonal antibody, or an antigen binding fragment thereof, which is capable of binding specifically to human VSIG4. In an embodiment, said antibody is capable of binding both the long form of human VSIG4 and the short form of VSIG4. In an embodiment, the long form of human VSIG4 protein has the sequence set forth in SEQ ID No. 1. In an embodiment, the short form of human VSIG4 protein has the sequence set forth in SEQ ID No. 2.

Anti-VSIG4 monoclonal antibodies as used herein include, but are not limited to, synthetic antibodies, recombinantly produced antibodies, multispecific antibodies (including bi-specific antibodies), human antibodies, humanised antibodies, camelised antibodies, chimeric antibodies, intrabodies, anti-idiotypic (anti-Id) antibodies, and functional fragments of any of the above. Anti-VSIG4 monoclonal antibodies can be of human or non-human origin. Examples of anti-VSIG4 antibodies of non-human origin include but are not limited to, those of mammalian origin (e.g., simians, rodents, goats, and rabbits). Because every structure of the human antibody originates from a human, there is only low probability of having an immune response compared to a conventional humanised antibody or mouse antibody, and thus it has an advantage that it does not cause any undesirable immune response when administered to a human. Therefore, it can be very advantageously used as an antibody for treatment. Accordingly, anti-VSIG4 monoclonal antibodies for therapeutic use in humans are preferably humanised or fully human. More preferably, they are fully human.

According to one embodiment of the present invention, the antibody described herein is a human antibody specifically binding to VSIG4 which was produced by the present inventors according to biopanning of a naive human single chain Fv library by phage display method.

In phage display methods, functional antibody domains are displayed on the surface of phage particles which carry the polynucleotide sequences encoding them. In particular, DNA sequences encoding V_{H} and V_{L} domains are amplified from animal cDNA libraries (e.g., human or murine cDNA libraries of affected tissues). The DNA encoding the V_{H} and V_{L} domains are recombined together with an scFv linker by PCR and cloned into a phagemid vector. The vector is electroporated in E. coli and the E. coli is infected with helper phage. Phage used in these methods are typically filamentous phage including fd and M13 and the V_{H} and V_{L} domains are usually recombinantly fused to either the phage gene III or gene VIII. Phage expressing an antigen binding domain that binds to a particular antigen can be selected or identified with antigen, e.g., using Labelled antigen or antigen bound or captured to a solid surface or bead. Examples of phage display methods that can be used to make the antibodies provided herein include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT/GB91/01134; WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)₂ fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043.

To generate whole antibodies, PCR primers including VH or VL nucleotide sequences, a restriction site, and a flanking sequence to protect the restriction site can be used to amplify the V_{H} or V_{L} sequences in scFv clones. Utilising cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lambda constant regions. The VH and VL domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

The antibody produced according to the above method are antibodies with enhanced affinity to the antigen. The term “affinity” indicates a property of specifically recognising and binding to a specific antigen site, and, together with specificity of an antibody for an antigen, the high affinity is an important factor in an immune reaction. In the present invention, humanised heavy chain library cells are produced by random mutation of a heavy chain variable region, and a colony lift assay was carried out for the library cells to select first variant clones having high antigen binding property. By carrying out competitive ELISA for the selected clones, affinity of each clone was examined. Other than this method, various methods for measuring the affinity for an antigen may be employed, and the surface plasmon resonance technology is one example of those methods.

In an embodiment, the anti-VSIG4 monoclonal antibody disclosed herein binds specifically to an epitope within the VSIG4 protein. Specifically, the epitope bound by the present antibody can be identified by determining which VSIG4 residues abolish antibody binding when mutated. In one embodiment, VSIG4 is the long variant. In another embodiment, VSIG4 is the short variant.

Preferably, the antibody disclosed herein is an antibody which binds to at least one amino acid in one or more epitope, the epitope being selected in the group consisting of:
a) an epitope M1 comprising residues E24, V25, E27, V29, and/or T30 of the sequence set forth in SEQ ID No. 2;
b) an epitope M2 comprising residues D36, N38, L39, and/or T42 of the sequence set forth in SEQ ID No. 2;
c) an epitope M3 comprising residues Q59, G61, S62, D63, and/or V65 of the sequence set forth in SEQ ID No. 2;
d) an epitope M4 comprising residues I77, A80, Y82, and/or Q83 of the sequence set forth in SEQ ID No. 2;
e) an epitope M5 comprising residues H87, H90, K91, and/or V92 of the sequence set forth in SEQ ID No. 2;
f) an epitope M6 comprising residues S97, Q99, S101, and/or T102 of the sequence set forth in SEQ ID No. 2;
g) an epitope M7 comprising residues R108, S109, H110, T112, and/or E114 of the sequence set forth in SEQ ID No. 2;
h) an epitope M8 comprising residues T119, P120, D121, N123, Q124, and/or V125 of the sequence set forth in SEQ ID No. 2.

More preferably, the antibody disclosed herein is an antibody which binds at least one of the amino acids in M7 and/or at least one of the amino acids in M8.

The determination of the binding of the anti-VSIG4 antibody to the epitope can be performed by any method or technique known to the person skilled in the art such as, without Limitation, radioactivity, Biacore, ELISA, flow cytometry, etc, or according to a method such as described in the present specification.

In an embodiment, the anti-VSIG4 monoclonal antibody disclosed herein comprises three heavy-chain CDRS and three light-chain CDRs. Preferably, the antibody comprises a heavy chain and a light chain, wherein the heavy chain comprises three heavy-chain CDRS and the light chain comprises three light-chain CDRs.

Preferably, the antibody disclosed herein comprises three heavy-chain CDRS and three heavy-chain CDRs, wherein the sequence of each CDR is selected in the group of sequences set forth in SEQ ID Nos. 3-14.

In an embodiment, the anti-VSIG4 antibody comprises three heavy-chain CDRs comprising sequences selected in the group consisting of SEQ ID Nos. 3, 4, 5, 9, 10, and 11.

In an embodiment, the anti-VSIG4 antibody comprises three light-chain CDRs comprising sequence selected in the group consisting of SEQ ID Nos. 6, 7, 8, 12, 13, and 14.

A preferred embodiment provides an anti-VSIG4 antibody having a heavy chain comprising three heavy-chain CDRs comprising sequences selected in the group consisting of SEQ ID Nos. 3, 4, 5, 9, 10, and 11.

Another preferred embodiment provides an anti-VSIG4 antibody having a light chain comprising three light-chain CDRs comprising sequences selected in the group consisting of SEQ ID Nos. 6, 7, 8, 12, 13, and 14.

In another preferred embodiment, the anti-VSIG4 antibody comprises three heavy-chain CDRs, the heavy-chain CDRs comprising sequences selected in the group consisting of SEQ ID Nos. 3, 4, 5, 9, 10, and 11; and three light-chain CDRs, the light-chain CDRs comprising sequences selected in the group consisting of SEQ ID Nos. 6, 7, 8, 12, 13, and 14.

In yet another preferred embodiment, the anti-VSIG4 antibody comprises a heavy chain, the heavy chain comprising three heavy-chain CDRs, wherein the heavy-chain CDRs comprises sequences selected in the group consisting of SEQ ID Nos. 3, 4, 5, 9, 10, and 11; and a light chain, the light chain comprising three light-chain CDRs, wherein the light-chain CDRs comprises sequences selected in the group consisting of SEQ ID Nos. 6, 7, 8, 12, 13, and 14.

More preferably, the antibody disclosed herein is selected in the group consisting of:
a) an antibody comprising the three heavy-chain CDRs of sequences SEQ ID Nos. 3, 4, and 5 and the three light-chain CDRs of sequences SEQ ID Nos. 6, 7, and 8; and
b) an antibody comprising the three heavy-chain CDRs of sequences SEQ ID Nos. 9, 10, and 11 and the three light-chain CDRs of sequences SEQ ID Nos. 12, 13, and 14.

In a preferred, but not Limitative, embodiment, the antibody of the invention is selected in the group consisting of:
a) an antibody comprising a heavy chain variable domain of sequence SEQ ID No. 45 or any sequence exhibiting at least 80%, 85%, 90%, 95% or 98% identity with SEQ ID No. 45, and the three light-chain CDRs of sequences SEQ ID Nos. 6, 7, and 8; and
b) an antibody comprising, or consisting of, a heavy chain variable domain of sequence SEQ ID No. 47 or any sequence exhibiting at least 80%, 85%, 90%, 95% or 98% identity with SEQ ID No. 47, and the three light-chain CDRs of sequences SEQ ID Nos. 12, 13, and 14.

By “any sequence exhibiting at least 80%, preferably 85%, 90%, 95% or 98% identity with SEQ ID No. 45”, it is intended to refer to a sequence exhibiting the three heavy-chain CDRs SEQ ID Nos. 3, 4, and 5 and, in addition, exhibiting at least 80%, preferably 85%, 90%, 95% or 98%, identity with the full sequence SEQ ID No. 45 outside the sequences corresponding to the CDRs (i.e. SEQ ID Nos. 3, 4, and 5), wherein “outside the sequences corresponding to the CDRs” is intended for “excepting the sequences corresponding to the CDRs”.

In another preferred, but not Limitative, embodiment, the antibody of the invention is selected in the group consisting of:
a) an antibody comprising a light chain variable domain of sequence SEQ ID No. 46 or any sequence exhibiting at least 80%, 85%, 90%, 95% or 98% identity with SEQ ID No. 46, and the three heavy-chain CDRs of sequences SEQ ID Nos. 3, 4, and 5;
b) an antibody comprising a light chain variable domain of sequence SEQ ID No. 48 or any sequence exhibiting at least 80%, 85%, 90%, 95% or 98% identity with SEQ ID No. 48, and the three heavy-chain CDRs of sequences SEQ ID Nos. 9, 10, and 11.

By “any sequence exhibiting at least 80%, preferably 85%, 90%, 95% or 98% identity with SEQ ID No. 46”, it is intended to refer to the sequences exhibiting the three light-chain CDRs SEQ ID Nos. 6, 7, and 8 and, in addition, exhibiting at least 80%, preferably 85%, 90%, 95% or 98%, identity with the full sequence SEQ ID No. 46 outside the sequences corresponding to the CDRs (i.e., SEQ ID Nos. 6, 7, and 8).

An embodiment of the disclosure relates to an antibody recognising VSIG4 and selected in the group consisting of:
a) an antibody comprising a heavy chain variable domain of sequence SEQ ID No. 45 or any sequence exhibiting at least 80%, 85%, 90%, 95% or 98% identity with SEQ ID No. 45 and a light chain variable domain of sequence SEQ ID No. 46 or any sequence exhibiting at least 80%, 85%, 90%, 95% or 98% identity with SEQ ID No. 46; and
b) an antibody comprising a heavy chain variable domain of sequence SEQ ID No. 47 or any sequence exhibiting at least 80%, 85%, 90%, 95% or 98% identity with SEQ ID No. 47 and a light chain variable domain of sequence SEQ ID No. 48 or any sequence exhibiting at least 80%, 85%, 90%, 95% or 98% identity with SEQ ID NO. 48.

The monoclonal antibody specifically binding to VSIG4 according to one embodiment of the present invention, or an antigen-binding fragment thereof is preferably an antibody selected in the group consisting of:
a) an antibody comprising a heavy chain variable region described by the amino acid sequence of SEQ ID NO: 45 and a light chain variable region described by the amino acid sequence of SEQ ID NO: 46; and
b) an antibody a heavy chain variable region described by the amino acid sequence of SEQ ID NO: 47 and a light chain variable region described by the amino acid sequence of SEQ ID NO: 48.

For more clarity, the following Table 2 illustrates the sequences (CDRs, frameworks, V_{H}, and V_{L}) of the preferred antibodies and the epitopes bound by each of these antibodies.

TABLE 2
Anti-VSIG4 antibodies

| Mab | CDRs | | | FR | V_{H} and V_{L} | Epitope |
|---|---|---|---|---|---|---|
| SA2281 | V_{H} CDR 1.2281 | GFTFSTYA | SEQ ID NO. 3 | H-FR1. 2281: SEQ ID NO. 21 | V_{H}2281: | M7 |
| | V_{H} CDR 2.2281 | VSHDGTYK | SEQ ID NO. 4 | H-FR2. 2281: SEQ ID NO. 22 | SEQ ID NO. 45 | |
| | V_{H} CDR 3.2281 | GRHAAGSSFDY | SEQ ID NO. 5 | H-FR3. 2281: SEQ ID NO. 23 | V_2281: | |
| | V_{L} CDR 1.2281 | RDISNY | SEQ ID NO. 6 | H-FR4. 2281: SEQ ID NO. 24 | SEQ ID NO. 46 | |
| | V_{L} CDR 2.2281 | KTS | SEQ ID NO. 7 | L-FR1. 2281: SEQ ID NO. 25 | | |
| | V_{L} CDR 3.2281 | QQSYRTPYT | SEQ ID NO. 8 | L-FR2. 2281: SEQ ID NO. 26 | | |
| | | | | L-FR3. 2281: SEQ ID NO. 27 | | |
| | | | | L-FR4. 2281: SEQ ID NO. 28 | | |
| | | | | H-FR1. 2297: SEQ ID NO. 29 | | |
| SA2297 | V_{H} CDR 1.2297 | GFIFSDYY | SEQ ID NO. 9 | H-FR2. 2297: SEQ ID NO. 30 | V_{H}2297: | M7/M8 |
| | V_{H} CDR 2.2297 | ISPSGYTI | SEQ ID NO. 10 | H-FR3. 2297: SEQ ID NO. 31 | SEQ ID NO. 47 | |
| | V_{H} CDR 3.2297 | ARRFSSSWSGYFDY | SEQ ID NO. 11 | H-FR4. 2297: SEQ ID NO. 32 | V_2297: | |
| | V_{L} CDR 1.2297 | QNINRW | SEQ ID NO. 12 | L-FR1. 2297: SEQ ID NO. 33 | SEQ ID NO.48 | |
| | V_{L} CDR 2.2297 | KAF | SEQ ID NO. 13 | L-FR2. 2297: SEQ ID NO. 34 | | |
| | V_{L} CDR 3.2297 | QQYLSYSSSI | SEQ ID NO. 14 | L-FR3. 2297: SEQ ID NO. 35 | | |
| | | | | L-FR4. 2297: SEQ ID NO. 36 | | |
| SA3981 | V_{H} CDR 1.3981 | GFTFSDYY | SEQ ID NO. 15 | H-FR1. 3981: SEQ ID NO. 37 | V_{H}3981: | M7 |
| | V_{H} CDR 2.3981 | ISSSGSTI | SEQ ID NO. 16 | H-FR2. 3981: SEQ ID NO. 38 | SEQ ID NO. 49 | |
| | V_{H} CDR 3.3981 | ARGYYYGMDV | SEQ ID NO. 17 | H-FR3. 3981: SEQ ID NO. 39 | V13981: | |
| | V_{L} CDR 1.3981 | SSDVGDYNL | SEQ ID NO. 18 | H-FR4. 3981: SEQ ID NO. 40 | SEQ ID NO.50 | |
| | V_{L} CDR 2.3981 | EVS | SEQ ID NO. 19 | L-FR1. 3981: SEQ ID NO. 41 | | |
| | V_{L} CDR 3.3981 | SSYKTGDTWL | SEQ ID NO. 20 | L-FR2. 3981: SEQ ID NO. 42 | | |
| | | | | L-FR3. 3981: SEQ ID NO. 43 | | |
| | | | | L-FR4. 3981: SEQ ID NO. 44 | | |

Within a range in which VSIG4 can be specifically recognised, the monoclonal antibody of the present invention or an antigen-binding fragment thereof may include not only the sequence of anti-VSIG4 antibody of the present invention, which is described in the present specification, but also a biological equivalent thereof. For example, to have further improvement of the binding affinity and/or other biological characteristics of an antibody, additional changes can be made on the amino acid sequence of an antibody. Included in those modifications are deletion, insertion, and/or substitution of the amino acid sequence of an antibody, for example. Those modifications of an amino acid are made based on relative similarity among side-chain substituents of an amino acid, for example, hydrophobicity, hydrophilicity, charge, size, or the like. Based on the analysis of the size, shape, and type of the side-chain substituents of an amino acid, it is found that all of arginine, lysine, and histidine are a residue with positive charge; alanine, glycine, and serine have a similar size; and phenylalanine, tryptophan, and tyrosine have a similar shape. Accordingly, it can be said based on those considerations that, biologically, arginine, lysine, and histidine; alanine, glycine, and serine; and phenylalanine, tryptophan, and tyrosine are functional equivalents.

In an embodiment, the anti-VSIG4 monoclonal antibodies described herein can be in the form of full-length antibodies, multiple chain or single chain antibodies, fragments of such antibodies that selectively bind to VSIG4 (including but not limited to Fab, Fab′, (Fab′)₂, Fv, and scFv), surrobodies (including surrogate light chain construct), single domain antibodies, humanised antibodies, camelised antibodies and the like. They also can be of, or derived from, any isotype, including, for example, IgA (e.g., Ig1l or IgA2), IgD, IgE, IgG (e.g., IgG1, IgG2, IgG3 or IgG4), or IgM. In some embodiments, the anti-VSIG4 antibody is an IgG (e.g., IgG1, IgG2, IgG3 or IgG4). In an embodiment, the antibody further comprises a human constant region. In a further embodiment, the human constant region is selected from the group consisting of IgG1, IgG2, IgG2, IgG3 and IgG4. In a still further specific embodiment, the human constant region is IgG1. Furthermore, the heavy chain constant region has gamma (γ), mu (μ), alpha (α), delta (δ) and epsilon (ε) types, and, as a subclass, it has gamma1 (γ1), gamma2 (γ2), gamma3 (γ3), gamma4 (γ4), alpha1 (α1) and alpha2 (α2). The light chain constant region has kappa (κ) and lambda (λ) types.

Anti-VSIG4 antibodies include labelled antibodies, useful in diagnostic applications. The antibodies can be used diagnostically, for example, to detect expression of a target of interest in specific cells, tissues, or serum; or to monitor the development or progression of an immunologic response as part of a clinical testing procedure to, e.g., determine the efficacy of a given treatment regimen. Detection can be facilitated by coupling the antibody to a detectable substance or “label.” A label can be conjugated directly or indirectly to an anti-VSIG4 antibody of the disclosure. The label can itself be detectable (e.g., radioisotope labels, isotopic labels, or fluorescent labels) or, in the case of an enzymatic label, can catalyse chemical alteration of a substrate compound or composition which is detectable. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. The detectable substance can be coupled or conjugated either directly to the antibody (or fragment thereof) or indirectly, through an intermediate (such as, for example, a linker known in the art) using techniques known in the art. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, acetylcholinesterase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride, dimethylamine-1-napthalenesulfonyl chloride, or phycoerythrin and the like; an example of a luminescent material includes luminol; examples of bioluminescent materials include luciferase, luciferin, and aequorin; examples of suitable isotopic materials include ¹³C, ¹⁵N, and deuterium; and examples of suitable radioactive material include ¹²⁵I, ¹³¹I, ¹¹¹In or ⁹⁹Tc.

### Bispecific Antibodies

In addition, the present disclosure provides a multi-specific antibody including the monoclonal anti-VSIG4 antibody disclosed herein or an antigen-binding fragment thereof.

The above multi-specific antibody in the present invention can preferably be a bi-specific antibody, but not limited thereto.

The multi-specific antibody according to the present invention preferably has the form in which the anti-VSIG4 antibody described herein is bound to an antibody having a binding property for an immunoeffector cell-specific target molecule, or a fragment thereof. The immunoeffector cell-specific target molecule is preferably an immune checkpoint, but it is not limited thereto. Examples of immunoeffector cell-specific target molecules include e.g., PD-1, PD-L1, CTLA-4, TIM-3, TIGIT, BTLA, KIR, A2aR, VISTA, B7-H3, TCR/CD3, CD16 (FcγRIIIa) CD44, Cd56, CD69, CD64 (FcγRI), CD89 and CD11b/CD18 (CR3).

The multi-specific antibody is an antibody which can simultaneously recognise different multi (bi or higher) epitopes of the same antigen or two or more separate antigens, and the antibodies belonging to multi-specific antibody can be classified into scFv-based antibody, Fab-based antibody, IgG-based antibody, or the like. In case of a multi-specific, e.g., bi-specific, antibody, two signals can be simultaneously suppressed or amplified, and thus it can be more effective than a case in which one signal is suppressed/amplified. Compared to a case in which each signal is treated with a signal inhibitor for each, low-dose administration can be achieved and two signals can be suppressed/amplified at the same time in the same space.

Methods for producing a bi-specific antibody are widely known. Conventionally, recombination production of a bi-specific antibody is based on coexpression of a pair of heavy chain/light chain of two immunogloubulins under conditions at which two heavy chains have different specificity.

In case of a scFv-based bi-specific antibody, by combining VL and VH of different scFvs, a hybrid scFv-based is prepared in heterodimer form to give a diabody (Holliger et al., Proc. Natl. Acad. Sci. U.S.A., 90:6444, 1993), and, by connecting different scFvs to each other, tandem ScFv can be produced. By expressing CH1 and CL of Fab at the terminus of each scFv, a heterodimeric mini antibody can be produced (Muller et al., FEBS lett., 432:45, 1998). In addition, by substituting partial amino acids of CH3 domain as a homodimeric domain of Fc, a structural change into “knob into hole” form to have a heterodimer structure is made and those modified CH3 domains are expressed at the terminus of each different scFv, and thus a minibody in heterodimeric scFv form can be produced (Merchant et al., Nat. Biotechnol., 16:677, 1998).

In case of a Fab-based bi-specific antibody, according to combination of separate Fab′ for a specific antigen by utilising a disulfide bond or a mediator, the antibody can be produced in heterodimeric Fab form, and, by expressing scFv for a different antigen at the terminus of a heavy chain or a light chain of a specific Fab, the antigen valency of 2 can be obtained. In addition, by having a hinge region between Fab and scFv, the antigen valency of 4 can be obtained in homodimer form. In addition, a method of producing the followings is known in the pertinent art: a dual target bibody by which the antigen valency of 3 is obtained according to fusion of scFv for a different antigen at the light chain terminus and heavy chain terminus of Fab, a triple target bibody by which the antigen valency of 3 is obtained according to fusion of different scFvs to the light chain terminus and heavy chain terminus of Fab, and a triple target antibody F(ab′)₃ in simple form that is obtained by chemical fusion of three different Fabs.

In case of IgG-based bi-specific antibody, a method of producing bi-specific antibody by preparing hybrid hybridoma, so-called quadromas, based on re-hybridisation of mouse and rat hybridomas is known by Trion Pharma. In addition, a method of producing a bi-specific antibody in so-called “Holes and Knob” form, in which partial amino acids of the CH3 homodimeric domain of Fc in different heavy chains are modified while sharing the light chain part, is known (Merchant et al., Nat. Biotechnol., 16:677, 1998), and, other than the bi-specific antibody in heterodimer form, a method of producing (scFv)₄-IgG in homodimer form according to fusion of two different scFvs to the constant domain of the light chain and heavy chain of IgG instead of the variable domain, followed by expression, is known. Furthermore, it has been reported by ImClone Systems that, based on IMC-1C11 as a chimeric monoclonal antibody for human VEGFR-2, only a single variable domain for mouse platelet-derived growth factor receptor-α is fused to the amino terminus of the light chain of the antibody so as to produce a bi-specific antibody. Furthermore, an antibody having high antigen valency for CD20 has been reported by Rossi et al. based on so-called “dock and lock (DNL)” method using a dimerisation and docking domain (DDD) of protein kinase A (PKA) R subunit and an anchoring domain of PKA (Rossi et al., Proc. Natl. Acad. Sci. U.S.A., 103:6841, 2006).

### Antibody Derivatives

The anti-VSIG4 antibodies of the present invention can be further modified to contain additional non-proteinaceous moieties that are known in the art and readily available. In particular, included herein are anti-VSIG4 monoclonal antibodies which are derivatised, covalently modified, or conjugated to other molecules, for use in diagnostic and therapeutic applications. For example, but not by way of Limitation, derivatised antibodies include antibodies that have been modified, e.g., by glycosylation, acetylation, pegylation, phosphorylation, amidation, derivatisation by known protecting/blocking groups, proteolytic cleavage, linkage to a cellular ligand or other protein, etc. Any of numerous chemical modifications can be carried out by known techniques, including, but not limited to, specific chemical cleavage, acetylation, formylation, metabolic synthesis of tunicamycin, etc. Additionally, the derivative can contain one or more non-classical amino acids.

In particular, the monoclonal antibody of the present invention or an antigen-binding fragment thereof may be subjected to derivatisation as described above, notably by e.g., glycosylation and/or PEGylation, in order to enhance the residence time in a living body to which the antibody is administered.

As for the glycosylation and/or PEGylation, various patterns of glycosylation and/or PEGylation can be modified by a method well known in the art, as long as the function of the antibody of the present invention is maintained, and included in the antibody of the present invention are a variant monoclonal antibody in which various patterns of glycosylation and/or PEGylation are modified, or an antigen-binding fragment thereof.

Preferably, the moieties suitable for derivatisation of the antibody are water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-l,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, polypropylene oxide/ethylene oxide co-polymers, polyoxyethylated polyols (e.g., glycerol), polyvinyl alcohol, and mixtures thereof. The polymer may be of any molecular weight, and may be branched or unbranched. The number of polymers attached to the antibody may vary, and if more than one polymer are attached, they can be the same or different molecules. In general, the number and/or type of polymers used for derivatisation can be determined based on considerations including, but not limited to, the particular properties or functions of the antibody to be improved, whether the antibody derivative will be used in a therapy under defined conditions, etc.

In a specific example, the anti-VSIG4 antibodies of the present disclosure can be attached to Poly(ethyleneglycol) (PEG) moieties. In a specific embodiment, the antibody is an antibody fragment and the PEG moieties are attached through any available amino acid side-chain or terminal amino acid functional group located in the antibody fragment, for example any free amino, imino, thiol, hydroxyl or carboxyl group. Such amino acids can occur naturally in the antibody fragment or can be engineered into the fragment using recombinant DNA methods. See, for example U.S. Pat. No. 5,219,996. Multiple sites can be used to attach two or more PEG molecules. PEG moieties can be covalently linked through a thiol group of at least one cysteine residue located in the antibody fragment. Where a thiol group is used as the point of attachment, appropriately activated effector moieties, for example thiol selective derivatives such as maleimides and cysteine derivatives, can be used.

In a specific example, an anti-VSIG4 antibody conjugate is a modified Fab′ fragment which is PEGylated, i.e., has PEG (poly(ethyleneglycol)) covalently attached thereto, e.g., according to the method disclosed in EP0948544. See also Poly(ethyleneglycol) Chemistry, Biotechnical and Biomedical Applications, (J. Milton Harris (ed.), Plenum Press, New York, 1992); Poly(ethyleneglycol) Chemistry and Biological Applications, (J. Milton Harris and S. Zalipsky, eds., American Chemical Society, Washington D.C., 1997); and Bioconjugation Protein Coupling Techniques for the Biomedical Sciences, (M. Aslam and A. Dent, eds., Grove Publishers, New York, 1998); and Chapman, 2002, Advanced Drug Delivery Reviews 54:531-545. PEG can be attached to a cysteine in the hinge region. In one example, a PEG-modified Fab′ fragment has a maleimide group covalently linked to a single thiol group in a modified hinge region. A lysine residue can be covalently linked to the maleimide group and to each of the amine groups on the lysine residue can be attached a methoxypoly(ethyleneglycol) polymer having a molecular weight of approximately 20,000 Da. The total molecular weight of the PEG attached to the Fab′ fragment can therefore be approximately 40,000 Da.

In another embodiment, conjugates of an antibody and non-proteinaceous moiety that may be selectively heated by exposure to radiation are provided. In one embodiment, the non-proteinaceous moiety is a carbon nanotube (Kam et al, Proc. Natl. Acad. Sci. USA 102: 11600-11605 (2005)). The radiation may be of any wavelength, and includes, but is not limited to, wavelengths that do not harm ordinary cells, but which heat the non-proteinaceous moiety to a temperature at which cells proximal to the antibody-non-proteinaceous moiety are killed.

### Immunoconjugates

In another aspect, the present disclosure provides an immunoconjugate (interchangeably referred to as “antibody-drug conjugates,” or “ADCs”) comprising an anti-VSIG4 antibody as described herein, said antibody being conjugated to a cytotoxic agent.

Many cytotoxic agents have been isolated or synthesised and make it possible to inhibit the cells proliferation, or to destroy or reduce, if not definitively, at least significantly the tumour cells. However, the toxic activity of these agents is not limited to tumour cells, and the non-tumour cells are also affected and can be destroyed. More particularly, side effects are observed on rapidly renewing cells, such as haematopoietic cells or cells of the epithelium, in particular of the mucous membranes. In order to limit side effects on normal cells whilst retaining high cytotoxicity on tumour cells, immunoconjugates have been used for the local delivery of cytotoxic agents in the treatment of cancer (Lambert, J. (2005) Curr. Opinion in Pharmacology 5:543-549; Wu et al (2005) Nature Biotechnology 23(9): 1137-1146; Payne, G. (2003) i 3:207-212; Syrigos and Epenetos (1999) Anticancer Research 19:605-614; Niculescu-Duvaz and Springer (1997) Adv. Drug Deliv. Rev. 26:151-172; U.S. Pat. No. 4,975,278). Immunoconjugates allow for the targeted delivery of a drug moiety (i.e., the cytotoxic agent) to a tumour, and intracellular accumulation therein, where systemic administration of unconjugated drugs may result in unacceptable levels of toxicity to normal cells as well as the tumour cells sought to be eliminated (Baldwin et al, Lancet (Mar. 15, 1986) pp. 603-05; Thorpe (1985) “Antibody Carriers Of Cytotoxic Agents In Cancer Therapy: A Review,” in Monoclonal Antibodies '84: Biological And Clinical Applications (A. Pinchera et al., eds) pp. 475-506. Both polyclonal antibodies and monoclonal antibodies have been reported as useful in these strategies (Rowland et al., (1986) Cancer Immunol. Immunother. 21:183-87).

The cytotoxic agent used in the immunoconjugates disclosed herein may be, without limitation, a drug (i.e., “antibody-drug conjugate”), a toxin (i.e., “immunotoxin” or “antibody-toxin conjugate”), a radioisotope (i.e., “radioimmunoconjugate” or “antibody-radioisotope conjugate”), etc.

Preferably, the immunoconjugate is a binding protein linked to at least a drug or a medicament. Such an immunoconjugate is usually referred to as an antibody-drug conjugate (or “ADC”) when the binding protein is an antibody, or an antigen binding fragment thereof.

In a first embodiment, such drugs can be described regarding their mode of action. As non-limitative examples, it can be mentioned alkylating agents such as nitrogen mustard, alkyl-sulfonates, nitrosourea, oxazophorins, aziridines or imine-ethylenes, anti-metabolites, anti-tumour antibiotics, mitotic inhibitors, chromatin function inhibitors, anti-angiogenesis agents, anti-ooestrogens, anti-androgens, chelating agents, iron absorption stimulant, cyclooxygenase inhibitors, phosphodiesterase inhibitors, DNA inhibitors, DNA synthesis inhibitors, apoptosis stimulants, thymidylate inhibitors, T cell inhibitors, interferon agonists, ribonucleoside triphosphate reductase inhibitors, aromatase inhibitors, ooestrogen receptor antagonists, tyrosine kinase inhibitors, cell cycle inhibitors, taxane, tubulin inhibitors, angiogenesis inhibitors, macrophage stimulants, neurokinin receptor antagonists, cannabinoid receptor agonists, dopamine receptor agonists, granulocytes stimulating factor agonists, erythropoietin receptor agonists, somatostatin receptor agonists, LHRH agonists, calcium sensitizers, VEGF receptor antagonists, interleukin receptor antagonists, osteoclast inhibitors, radical formation stimulants, endothelin receptor antagonists, vinca alkaloid, anti-hormone or immunomodulators or any other new drug that fulfils the activity criteria of a cytotoxic or a toxin.

Such drugs are, for example, cited in VIDAL 2010, on the page devoted to the compounds attached to the cancerology and haematology column “Cytotoxics”, these cytotoxic compounds cited with reference to this document are cited here as preferred cytotoxic agents.

More particularly, without limitation, the following drugs are preferred according to the invention: mechlorethamine, chlorambucol, melphalen, chlorhydrate, pipobromen, prednimustin, disodic-phosphate, estramustine, cyclophosphamide, altretamine, trofosfamide, sulfofosfamide, ifosfamide, thiotepa, triethylenamine, altetramine, carmustine, streptozocin, fotemustin, lomustine, busulfan, treosulfan, improsulfan, dacarbazine, cis-platinum, oxaliplatin, lobaplatin, heptaplatin, miriplatin hydrate, carboplatin, methotrexate, pemetrexed, 5-fluoruracil, floxuridine, 5-fluorodeoxyuridine, capecitabine, cytarabine, fludarabine, cytosine arabinoside, 6-mercaptopurine (6-MP), nelarabine, 6-thioguanine (6-TG), chlorodesoxyadenosine, 5-azacytidine, gemcitabine, cladribine, deoxycoformycin, tegafur, pentostatin, doxorubicin, daunorubicin, idarubicin, valrubicin, mitoxantrone, dactinomycin, mithramycin, plicamycin, mitomycin C, bleomycin, procarbazine, paclitaxel, docetaxel, vinblastine, vincristine, vindesine, vinorelbine, topotecan, irinotecan, etoposide, valrubicin, amrubicin hydrochloride, pirarubicin, elliptinium acetate, zorubicin, epirubicin, idarubicin and teniposide, razoxin, marimastat, batimastat, prinomastat, tanomastat, ilomastat, CGS-27023A, halofuginon, COL-3, neovastat, thalidomide, CDC 501, DMXAA, L-651582, squalamine, endostatin, SU5416, SU6668, interferon-alpha, EMD121974, interleukin-12, IM862, angiostatin, tamoxifen, toremifene, raloxifene, droloxifene, iodoxyfene, anastrozole, letrozole, exemestane, flutamide, nilutamide, sprironolactone, cyproterone acetate, finasteride, cimitidine, bortezomid, velcade, bicalutamide, cyproterone, flutamide, fulvestran, exemestane, dasatinib, erlotinib, gefitinib, imatinib, lapatinib, nilotinib, sorafenib, sunitinib, retinoid, rexinoid, methoxsalene, methylaminolevulinate, aldesleukine, OCT-43, denileukin diflitox, interleukin-2, tasonermine, lentinan, sizofilan, roquinimex, pidotimod, pegademase, thymopentine, poly I:C, procodazol, Tic BCG, Corynebacterium parvum, NOV-002, ukrain, levamisole, 1311-chTNT, H-101, celmoleukin, interferon alfa2a, interferon alfa2b, interferon gamma1a, interleukin-2, mobenakin, Rexin-G, teceleukin, aclarubicin, actinomycin, arglabin, asparaginase, carzinophilin, chromomycin, daunomycin, leucovorin, masoprocol, neocarzinostatin, peplomycin, sarkomycin, solamargine, trabectedin, streptozocin, testosterone, kunecatechins, sinecatechins, alitretinoin, belotecan hydrocholoride, calusterone, dromostanolone, elliptinium acetate, ethinyl estradiol, etoposide, fluoxymesterone, formestane, fosfetrol, goserelin acetate, hexyl aminolevulinate, histrelin, hydroxyprogesterone, ixabepilone, leuprolide, medroxyprogesterone acetate, megesterol acetate, methylprednisolone, methyltestosterone, miltefosine, mitobronitol, nadrolone phenylpropionate, norethindrone acetate, prednisolone, prednisone, temsirrolimus, testolactone, triamconolone, triptorelin, vapreotide acetate, zinostatin stimalamer, amsacrine, arsenic trioxide, bisantrene hydrochloride, chlorambucil, chlortrianisene, cis-diamminedichloroplatinium, cyclophosphamide, diethylstilbestrol, hexamethylmelamine, hydroxyurea, lenalidomide, lonidamine, mechlorethanamine, mitotane, nedaplatin, nimustine hydrochloride, pamidronate, pipobroman, porfimer sodium, ranimustine, razoxane, semustine, sobuzoxane, mesylate, triethylenemelamine, zoledronic acid, camostat mesylate, fadrozole HCl, nafoxidine, aminoglutethimide, carmofur, clofarabine, cytosine arabinoside, decitabine, doxifluridine, enocitabine, fludarabne phosphate, fluorouracil, ftorafur, uracil mustard, abarelix, bexarotene, raltiterxed, tamibarotene, temozolomide, vorinostat, megastrol, clodronate disodium, levamisole, ferumoxytol, iron isomaltoside, celecoxib, ibudilast, bendamustine, altretamine, mitolactol, temsirolimus, pralatrexate, TS-1, decitabine, bicalutamide, flutamide, letrozole, clodronate disodium, degarelix, toremifene citrate, histamine dihydrochloride, DW-166HC, nitracrine, decitabine, irinoteacn hydrochloride, amsacrine, romidepsin, tretinoin, cabazitaxel, vandetanib, lenalidomide, ibandronic acid, miltefosine, vitespen, mifamurtide, nadroparin, granisetron, ondansetron, tropisetron, alizapride, ramosetron, dolasetron mesilate, fosaprepitant dimeglumine, nabilone, aprepitant, dronabinol, TY-10721, lisuride hydrogen maleate, epiceram, defibrotide, dabigatran etexilate, filgrastim, pegfilgrastim, reditux, epoetin, molgramostim, oprelvekin, sipuleucel-T, M-Vax, acetyl L-carnitine, donepezil hydrochloride, 5-aminolevulinic acid, methyl aminolevulinate, cetrorelix acetate, icodextrin, leuprorelin, metbylphenidate, octreotide, amlexanox, plerixafor, menatetrenone, anethole dithiolethione, doxercalciferol, cinacalcet hydrochloride, alefacept, romiplostim, thymoglobulin, thymalfasin, ubenimex, imiquimod, everolimus, sirolimus, H-101, lasofoxifene, trilostane, incadronate, gangliosides, pegaptanib octasodium, vertoporfin, minodronic acid, zoledronic acid, gallium nitrate, alendronate sodium, etidronate disodium, disodium pamidronate, dutasteride, sodium stibogluconate, armodafinil, dexrazoxane, amifostine, WF-10, temoporfin, darbepoetin alfa, ancestim, sargramostim, palifermin, R-744, nepidermin, oprelvekin, denileukin diftitox, crisantaspase, buserelin, deslorelin, lanreotide, octreotide, pilocarpine, bosentan, calicheamicin, maytansinoids and ciclonicate.

For more detail, the person skilled in the art may refer to the manual edited by the “Association Françaíse des Enseignants de Chimie Thérapeutique” and entitled “Traité de chimie thérapeutique, vol. 6, Médicaments antitumouraux et perspectives dans le traitement des cancers, edition TEC & DOC, 2003”.

Alternatively, the immunoconjugate may comprise a binding protein linked to at least a radioisotope. Such an immunoconjugate is usually referred to as an antibody-radioisotope conjugate (or “ARC”) when the binding protein is an antibody, or an antigen binding fragment thereof.

For selective destruction of the tumour, the antibody may comprise a highly radioactive atom. A variety of radioactive isotopes are available for the production of ARC such as, without Limitation, At²¹¹, C¹¹, N¹⁵, O¹⁷, F¹⁹, I¹²³, I¹³¹, I¹²⁵, In¹¹, Y⁹⁰, Re¹⁸⁶, Re¹⁸⁸, Sm¹⁵³, tc⁹⁹m, Bi²¹², P³², Pb²¹², radioactive isotopes of Lu, gadolinium, manganese or iron.

Any methods or processes known by the person skilled in the art can be used to incorporate such radioisotope in the ARC (see, for example “Monoclonal Antibodies in Immunoscintigraphy”, Chatal, CRC Press 1989). As non-limitative examples, Tc⁹⁹m or I¹²³, Re¹⁸⁶, Re¹⁸⁸ and In¹¹¹ can be attached via a cysteine residue. Y⁹⁰ can be attached via a lysine residue. 1¹²³ can be attached using the IODOGEN method (Fraker et al (1978) Biochem. Biophys. Res. Commun. 80: 49-57).

Several examples can be mentioned to illustrate the knowledge of the person skilled in the art in the field of ARC such as Zevalin® which is an ARC composed of an anti-CD20 monoclonal antibody and In¹¹¹ or Y⁹⁰ radioisotope bound by a thiourea linker-chelator (Wiseman et at (2000) Eur. Jour. Nucl. Med. 27(7):766-77; Wiseman et al (2002) Blood 99(12):4336-42; Witzig et at (2002) J. Clin. Oncol. 20(10):2453-63; Witzig et al (2002) J. Clin. Oncol. 20(15):3262-69); or Mylotarg® which is composed of an anti-CD33 antibody linked to calicheamicin, (U.S. Pat. Nos. 4,970,198; 5,079,233; 5,585,089; 5,606,040; 5,693,762; 5,739,116; 5,767,285; 5,773,001). More recently, it can also be mentioned the ADC referred as Adcetris (corresponding to the Brentuximab vedotin) which has been recently accepted by the FDA in the treatment of Hodgkin's lymphoma (Nature, vol. 476, pp 380-381, 25 Aug. 2011).

In yet another embodiment of the disclosure, the immunoconjugate may comprise a binding protein linked to a toxin. Such an immunoconjugate is usually referred to as an antibody-toxin conjugate (or “ATC”) when the binding protein is an antibody, or an antigen binding fragment thereof.

Toxins are effective and specific poisons produced by living organisms. They usually consist of an amino acid chain whose molecular weight may vary between a couple of hundred (peptides) and one hundred thousand daltons (proteins). They may also be low-molecular organic compounds. Toxins are produced by numerous organisms, e.g., bacteria, fungi, algae and plants. Many of them are extremely poisonous, with a toxicity that is several orders of magnitude greater than the nerve agents.

Toxins used in ATC can include, without Limitation, all kind of toxins which may exert their cytotoxic effects by mechanisms including tubulin binding, DNA binding, or topoisomerase inhibition.

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

Small molecule toxins, such as dolastatins, auristatins, a trichothecene, and CC1065, and the derivatives of these toxins that have toxin activity, are also contemplated herein. Dolastatins and auristatins have been shown to interfere with microtubule dynamics, GTP hydrolysis, and nuclear and cellular division and have anticancer and antifungal activity.

The immunoconjugates described herein may further comprise a linker.

“Linker”, “Linker Unit”, or “link” means a chemical moiety comprising a covalent bond or a chain of atoms that covalently attaches a binding protein to at least one cytotoxic agent.

Linkers may be made using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Carbon-14-labelled 1-isothiocyanatobenzyl-3-methyldiethylene triaminepentaacetic acid (MX-DTPA) is an exemplary chelating agent for conjugation of cytotoxic agents to the addressing system. Other cross-linker reagents may be BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

The linker may be a “non-cleavable” or “cleavable” linker.

Preferably, the linker is a “cleavable linker” facilitating release of the cytotoxic agent in the cell. For example, an acid-labile linker, a peptidase-sensitive linker, a photolabile linker, a dimethyl linker or a disulfide-containing linker may be used. The linker is preferably cleaved under intracellular conditions, such that cleavage of the linker releases the cytotoxic agent from the binding protein in the intracellular environment.

For example, in some embodiments, the linker may be cleaved by a cleaving agent that is present in the intracellular environment (e.g., within a lysosome or endosome or caveolea). The linker can be, for example, a peptidyl linker that is cleaved by an intracellular peptidase or protease enzyme, including, but not limited to, a lysosomal or endosomal protease. Typically, the peptidyl linker is at least two amino acids long or at least three amino acids long. Cleaving agents can include cathepsins B and D and plasmin, all of which are known to hydrolyse dipeptide drug derivatives resulting in the release of active drug inside target cells. For example, a peptidyl linker that is cleavable by the thiol-dependent protease cathepsin-B, which is highly expressed in cancerous tissue, can be used (e.g., a Phe-Leu or a Gly-Phe-Leu-Gly linker). In specific embodiments, the peptidyl linker cleavable by an intracellular protease is a Val-Cit linker or a Phe-Lys linker. One advantage of using intracellular proteolytic release of the cytotoxic agent is that the agent is typically attenuated when conjugated and the serum stabilities of the conjugates are typically high.

In other embodiments, the cleavable linker is pH-sensitive, i.e., sensitive to hydrolysis at certain pH values. Typically, the pH-sensitive linker is hydrolysable under acidic conditions. For example, an acid-labile linker that is hydrolysable in the lysosome (e.g., a hydrazone, semicarbazone, thiosemicarbazone, cis-aconitic amide, orthoester, acetal, ketal, or the like) can be used. Such linkers are relatively stable under neutral pH conditions, such as those in the blood, but are unstable at below pH 5.5 or 5.0, the approximate pH of the lysosome. In certain embodiments, the hydrolysable linker is a thioether linker (such as, e.g., a thioether attached to the therapeutic agent via an acylhydrazone bond.

In yet other embodiments, the linker may be cleaved under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate), and SMPT (N-succinimidyl-oxycarbonyl-alpha-methyl-alpha-(2-pyridyl-dithio)toluene).

Non-cleavable linkers by contrast have no obvious drug release mechanism. Immunoconjugates comprising such non-cleavable linkers rely on the complete lysosomal proteolytic degradation of the antibody that releases the cytotoxic agent after internalisation.

As an example of an immunoconjugate comprising a non-cleavable linker, the immunoconjugate trastuzumab-emtansine (TDM1) can be mentioned, which combines trastuzumab with a linked chemotherapeutic agent, maytansin (Cancer Research 2008; 68: (22). Nov. 15, 2008).

In a preferred embodiment, the immunoconjugate disclosed herein may be prepared by any method known by the person skilled in the art such as, without limitation, i) reaction of a nucleophilic group of the antigen binding protein with a bivalent linker reagent followed by reaction with the cytotoxic agent or ii) reaction of a nucleophilic group of a cytotoxic agent with a bivalent linker reagent followed by reaction with the nucleophilic group of the antigen binding protein.

Nucleophilic groups on antigen binding protein include, without limitation, N-terminal amine groups, side chain amine groups, e.g. lysine, side chain thiol groups, and sugar hydroxyl or amino groups when the antigen binding protein is glycosylated. Amine, thiol, and hydroxyl groups are nucleophilic and capable of reacting to form covalent bonds with electrophilic groups on linker moieties and linker reagents including, without limitation, active esters such as NHS esters, HOBt esters, haloformates, and acid halides; alkyl and benzyl halides such as haloacetamides; aldehydes, ketones, carboxyl, and maleimide groups. The antigen binding protein may have reducible interchain disulfides, i.e. cysteine bridges. The antigen binding proteins may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into the antigen binding protein through any reaction known by the person skilled in the art. As non-Limitative example, reactive thiol groups may be introduced into the antigen binding protein by introducing one or more cysteine residues.

Immunoconjugates may also be produced by modification of the antigen binding protein to introduce electrophilic moieties, which can react with nucleophilic substituents on the linker reagent or cytotoxic agent. The sugars of glycosylated antigen binding protein may be oxidised to form aldehyde or ketone groups which may react with the amine group of linker reagents or cytotoxic agent. The resulting imine Schiff base groups may form a stable linkage, or may be reduced to form stable amine linkages. In one embodiment, reaction of the carbohydrate portion of a glycosylated antigen binding protein with either galactose oxidase or sodium meta-periodate may yield carbonyl (aldehyde and ketone) groups in the protein that can react with appropriate groups on the drug. In another embodiment, proteins containing N-terminal serine or threonine residues can react with sodium meta-periodate, resulting in production of an aldehyde in place of the first amino acid.

### Chimeric Antigen Receptors

The present disclosure further provides a CAR (chimeric antigen receptor) protein including i) the antibody of the present invention; ii) a transmembrane domain, and; iii) an intracellular signalling domain characterised by causing T cell activation according to binding of the antibody of above i) to an antigen.

In the present invention, the CAR protein is characterised in that it is constituted by the monoclonal antibody of the present invention, a publicly known transmembrane domain, and an intracellular signalling domain

As described herein, the term “CAR (chimeric antigen receptor)” refers to a non-natural receptor capable of providing specificity for a specific antigen to an immunoeffector cell. In general, the CAR indicates a receptor that is used for providing the specificity of a monoclonal antibody to T cells. The CAR is generally constituted with an extracellular domain, a transmembrane domain and an intracellular domain. The extracellular domain includes an antigen recognition region, and, in the present invention, the antigen recognition site is VSIG4-specific antibody. The VSIG4-specific antibody is as described in the above, and the antibody used in CAR is preferably in the form of an antibody fragment. It is more preferably in the form of Fab or scFv, but not limited thereto.

Furthermore, the transmembrane domain of CAR has the form in which it is connected to the extracellular domain, and it may be originated from either natural or synthetic form. When it is originated from natural form, it may be originated from a membrane-bound or transmembrane protein, and it can be a part originated from transmembrane domains of various proteins like alpha, beta or zeta chain of T cell receptor, CD28, CD3 epsilon, CD45, CD4, CD5, CDS, CD9, CD16, CD22, CD33, CD37, CD64, CD80, CD86, CD134, CD137, CD154 or CD8. Sequences of those transmembrane domains can be obtained from documents that are well known in the art, in which the transmembrane domain of a transmembrane protein is described well, but it is not limited thereto.

The CAR of the present invention is the part of intracellular CAR domain, and it is connected to the transmembrane domain. The intracellular domain of the present invention may include an intracellular signalling domain, which is characterised by having a property of causing T cell activation, preferably T cell proliferation, upon binding of an antigen to the antigen recognition site of CAR. The intracellular signalling domain is not particularly limited in terms of the type thereof as long as it can cause the T cell activation upon binding of an antigen to the antigen recognition site of CAR present outside a cell, and various kinds of an intracellular signalling domain can be used. Examples thereof include immunoreceptor tyrosine based activation motif (ITAM), and the ITAM may include those originating from CD3 zeta (ξ,), FcR gamma, FcR beta, CD3 gamma, CD3 delta, CD3 epsilon, CDS, CD22, CD79a, CD79b, CD66d or FcεRIγ, but not limited thereto.

Furthermore, it is preferable that the intracellular domain of the CAR of the present invention additionally comprises a costimulatory domain with the intracellular signalling domain, but not limited thereto. The costimulatory domain is a part which is comprised in the CAR of the present invention and plays a role of transferring a signal to T cells in addition to the signal from the intracellular signalling domain, and it indicates the intracellular part of CAR including the intracellular domain of a costimulatory molecule.

The costimulatory molecule means, as a cell surface molecule, a molecule required for having a sufficient reaction of lymphocytes for an antigen, and examples thereof include CD27, CD28, 4-1BB, OX40, CD30, CD40, PD-1, ICOS, LFA-1 (lymphocyte function-associated antigen-1), CD2, CD7, LIGHT, NKG2C, and B7-H3, but not limited thereto. The costimulatory domain can be an intracellular part of a molecule that is selected from the group consisting of those costimulatory molecules and a combination thereof.

Furthermore, selectively, a short oligopeptide or polypeptide linker may link the intracellular domain and transmembrane domain of CAR. Although this linker may be included in the CAR of the present invention, it is not particularly limited in terms of the linker length as long as it can induce the T cell activation via the intracellular domain binding of an antigen to an extracellular antibody.

Nucleic Acids and Expression Systems

The present disclosure encompasses polynucleotides encoding immunoglobulin light and heavy chain genes for antibodies, notably anti-VSIG4 antibodies, vectors comprising such nucleic acids, and host cells capable of producing the antibodies of the disclosure. Also provided herein are polynucleotides that hybridise under high stringency, intermediate or lower stringency hybridisation conditions, e.g., as defined supra, to polynucleotides that encode an antibody or modified antibody provided herein.

In a first aspect, the present disclosure relates to one or more polynucleotides encoding an antibody, notably an antibody capable of binding specifically to VSIG4, or a fragment thereof, as described above. The present disclosure notably provides a polynucleotide encoding the heavy chain variable region and/or the light chain variable region of the monoclonal antibody, or an antigen-binding fragment thereof. More specifically, in certain embodiments, nucleic acid molecules provided herein comprise or consist of a nucleic acid sequence encoding the heavy chain variable region and light chain variable region disclosed herein, or any combination thereof (e.g., as a nucleotide sequence encoding an antibody provided herein, such as e.g., a full-length antibody, heavy and/or light chain of an antibody, or a single chain antibody provided herein).

In an embodiment, the polynucleotide encodes three heavy-chain CDRs of the anti-VSIG4 antibody described herein. In an embodiment, the polynucleotide encodes three light-chain CDRs of the anti-VSIG4 antibody described herein. In an embodiment, the polynucleotide encodes three heavy-chain CDRs and three light-chain CDRs of the anti-VSIG4 antibody described herein. Another embodiment provides a couple of polynucleotides, wherein the first polynucleotide encodes three heavy-chain CDRs of the anti-VSIG4 antibody described herein; and the second polynucleotide encodes three light-chain CDRs of the same anti-VSIG4 antibody described herein.

In an embodiment, the polynucleotide encodes the heavy-chain variable region of the anti-VSIG4 antibody described herein. In an embodiment, the polynucleotide encodes the light-chain variable region of the anti-VSIG4 antibody described herein. In an embodiment, the polynucleotide encodes the heavy-chain variable region and the light-chain variable region of the anti-VSIG4 antibody described herein. Another embodiment provides a couple of polynucleotides, wherein the first polynucleotide encodes the heavy-chain variable region of the anti-VSIG4 antibody described herein; and the second polynucleotide encodes the light-chain variable region of the same anti-VSIG4 antibody described herein.

In an embodiment, the polynucleotide encodes the heavy-chain of the anti-VSIG4 antibody described herein. In an embodiment, the polynucleotide encodes the light-chain of the anti-VSIG4 antibody described herein. In an embodiment, the polynucleotide encodes the heavy-chain and the light-chain of the anti-VSIG4 antibody described herein. Another embodiment provides a couple of polynucleotides, wherein the first polynucleotide encodes the heavy-chain of the anti-VSIG4 antibody described herein; and the second polynucleotide encodes the light-chain of the same anti-VSIG4 antibody described herein.

In an embodiment, the polynucleotide encodes the heavy chain of an anti-VSIG4 antibody SA2281 described above is provided. Preferably, said heavy chain comprises three heavy-chain CDRs of sequence SEQ ID Nos. 3, 4, and 5. More preferably, said heavy chain comprises a heavy chain comprising the variable region of sequence SEQ ID No. 45.

In another embodiment, the polynucleotide encodes the light chain of an anti-VSIG4 antibody SA2281 described above. Preferably, said light chain comprises three light-chain CDRs of sequence SEQ ID Nos. 6, 7, and 8. More preferably, said light chain comprises a light chain comprising the variable region of sequence SEQ ID No. 46.

In another embodiment, a polynucleotide encoding the heavy chain of an anti-VSIG4 antibody SA2297 described above is provided. Preferably, said heavy chain comprises three heavy-chain CDRs of sequence SEQ ID Nos. 9, 10, and 11. More preferably, said heavy chain comprises a heavy chain comprising the variable region of sequence SEQ ID No. 47.

In another embodiment, the polynucleotide encodes the light chain of an anti-VSIG4 antibody SA2297 described above. Preferably, said light chain comprises three light-chain CDRs of sequence SEQ ID Nos. 12, 13, and 14. More preferably, said light chain comprises a light chain comprising the variable region of sequence SEQ ID No. 48.

Due to the codon degeneracy or in consideration of a codon preferred in an organism in which the light chain and heavy chain of human antibody or a fragment thereof is to be expressed, the polynucleotide encoding the light chain and heavy chain of the monoclonal antibody of the present invention or an antigen-binding fragment thereof can have various variations in the coding region within a range in which the amino acid sequence of the light chain and heavy chain of an antibody expressed from the coding region is not changed, and, even in a region other than the coding region, various changes or modifications can be made within a range in which the gene expression is not affected by them. The skilled person will easily understand that those variant genes also fall within the scope of the present invention. Namely, as long as a protein having the equivalent activity is encoded by the polynucleotide of the present invention, one or more nucleic acid bases can be changed by substitution, deletion, insertion, or a combination thereof, and those also fall within the scope of the present invention. Sequence of the polynucleotide may be either a single chain or a double chain, and it may be either a DNA molecule or an RNA (mRNA) molecule.

According to the invention, a variety of expression systems may be used to express the antibody of the invention. In one aspect, such expression systems represent vehicles by which the coding sequences of interest may be produced and subsequently purified, but also represent cells which may, when transiently transfected with the appropriate nucleotide coding sequences, express an IgG antibody in situ.

The disclosure provides vectors comprising the polynucleotides described above. In one embodiment, the vector contains a polynucleotide encoding a heavy chain of the antibody of interest (e.g., an anti-VSIG4 antibody). In another embodiment, the polynucleotide encodes the light chain of the antibody of interest (e.g., an anti-VSIG4 antibody). In another embodiment, the polynucleotide encodes the heavy chain and the light chain of the antibody of interest (e.g., an anti-VSIG4 antibody). In yet another embodiment, a couple of polynucleotides are provided, wherein the first polynucleotide encodes the heavy chain of the antibody of interest (e.g., an anti-VSIG4 antibody), and the second polynucleotide encodes the light chain of the same antibody of interest (e.g., an anti-VSIG4 antibody).

The disclosure also provides vectors comprising polynucleotide molecules encoding fusion proteins, modified antibodies, antibody fragments, and probes thereof.

In order to express the heavy and/or light chain of the antibody of interest (e.g., an anti-VSIG4 antibody), the polynucleotides encoding said heavy and/or light chains are inserted into expression vectors such that the genes are operatively linked to transcriptional and translational sequences. In a preferred embodiment, these polynucleotides are cloned into two vectors.

“Operably linked” sequences include both expression control sequences that are contiguous with the gene of interest and expression control sequences that act in trans or at a distance to control the gene of interest. The term “expression control sequence” as used herein refers to polynucleotide sequences which are necessary to affect the expression and processing of coding sequences to which they are ligated. Expression control sequences include appropriate transcription initiation, termination, promoter and enhancer sequences; efficient RNA processing signals such as splicing and polyadenylation signals; sequences that stabilise cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

Polynucleotides of the invention and vectors comprising these molecules can be used for the transformation of a suitable host cell. The term “host cell”, as used herein, is intended to refer to a cell into which a recombinant expression vector has been introduced in order to express the antibody of interest (e.g., an anti-VSIG4 antibody). It should be understood that such terms are intended to refer not only to the particular subject cell but also to the progeny of such a cell. Because certain modifications may occur in succeeding generations due to either mutation or environmental influences, such progeny may not, in fact, be identical to the parent cell, but are still included within the scope of the term “host cell” as used herein.

Transformation can be performed by any known method for introducing polynucleotides into a cell host. Such methods are well known of the man skilled in the art and include dextran-mediated transformation, calcium phosphate precipitation, polybrene-mediated transfection, protoplast fusion, electroporation, encapsulation of the polynucleotide into liposomes, biolistic injection and direct microinjection of DNA into nuclei.

The host cell may be co-transfected with one or more expression vectors. For example, a host cell can be transfected with a vector encoding both the heavy chain and the light chain of the antibody of interest (e.g., an anti-VSIG4 antibody), as described above. Alternatively, the host cell can be transformed with a first vector encoding the heavy chain of the antibody of interest (e.g., an anti-VSIG4 antibody), and with a second vector encoding the light chain of said antibody. Mammalian cells are commonly used for the expression of a recombinant therapeutic immunoglobulins, especially for the expression of whole recombinant antibodies. For example, mammalian cells such as HEK293 or CHO cells, in conjunction with a vector, containing the expression signal such as one carrying the major intermediate early gene promoter element from human cytomegalovirus, are an effective system for expressing the humanised anti-VSIG4 antibody of the invention (Foecking et al., 1986, Gene 45:101; Cockett et al., 1990, Bio/Technology 8: 2).

In addition, a host cell may be chosen which modulates the expression of the inserted sequences, or modifies and processes the gene product in the specific fashion desired. Such modifications (e.g., glycosylation) and processing of protein products may be important for the function of the protein. Different host cells have features and specific mechanisms for the post-translational processing and modification of proteins and gene products. Appropriate cell lines or host systems are chosen to ensure the correct modification and processing of the expressed antibody of interest. Hence, eukaryotic host cells which possess the cellular machinery for proper processing of the primary transcript, glycosylation of the gene product may be used. Such mammalian host cells include, but are not limited to, CHO, COS, HEK293, NS/0, BHK, Y2/0, 3T3 or myeloma cells (all these cell lines are available from public depositories such as the Collection Nationale des Cultures de Microorganismes, Paris, France, or the American Type Culture Collection, Manassas, VA, U.S.A.).

For long-term, high-yield production of recombinant proteins, stable expression is preferred. In one embodiment of the invention, cell lines which stably express the antibody may be engineered. Rather than using expression vectors which contain viral origins of replication, host cells are transformed with DNA under the control of the appropriate expression regulatory elements, including promoters, enhancers, transcription terminators, polyadenylation sites, and other appropriate sequences known to the person skilled in art, and a selectable marker. Following the introduction of the foreign DNA, engineered cells may be allowed to grow for one to two days in an enriched media, and then are moved to a selective media. The selectable marker on the recombinant plasmid confers resistance to the selection and allows cells to stably integrate the plasmid into a chromosome and be expanded into a cell line. Other methods for constructing stable cell lines are known in the art. In particular, methods for site-specific integration have been developed. According to these methods, the transformed DNA under the control of the appropriate expression regulatory elements, including promoters, enhancers, transcription terminators, polyadenylation sites, and other appropriate sequences is integrated in the host cell genome at a specific target site which has previously been cleaved (Moele et al., Proc. Natl. Acad. Sci. U.S.A., 104(9): 3055-3060; U.S. Pat. Nos. 5,792,632; 5,830,729; 6,238,924; WO 2009/054985; WO 03/025183; WO 2004/067753).

A number of selection systems may be used according to the invention, including but not limited to the Herpes simplex virus thymidine kinase (Wigler et al., Cell 11:223, 1977), hypoxanthine-guanine phosphoribosyltransferase (Szybalska et al., Proc Natl Acad Sci USA 48: 202, 1992), glutamate synthase selection in the presence of methionine sulfoximide (Adv Drug Del Rev, 58: 671, 2006, and website or litreature of Lonza Group Ltd.) and adenine phosphoribosyltransferase (Lowy et al., Cell 22: 817, 1980) genes in tk, hgprt or aprt cells, respectively. Also, antimetabolite resistance can be used as the basis of selection for the following genes: dhfr, which confers resistance to methotrexate (Wigler et al., Proc Natl Acad Sci USA 77: 357, 1980); gpt, which confers resistance to mycophenolic acid (Mulligan et al., Proc Natl Acad Sci USA 78: 2072, 1981); neo, which confers resistance to the aminoglycoside, G-418 (Wu et al., Biotherapy 3: 87, 1991); and hygro, which confers resistance to hygromycin (Santerre et al., Gene 30: 147, 1984). Methods known in the art of recombinant DNA technology may be routinely applied to select the desired recombinant clone, and such methods are described, for example, in Ausubel et al., eds., Current Protocols in Molecular Biology, John Wiley & Sons (1993). The expression levels of an antibody can be increased by vector amplification. When a marker in the vector system expressing an antibody is amplifiable, an increase in the level of inhibitor present in the culture will increase the number of copies of the marker gene. Since the amplified region is associated with the gene encoding the IgG antibody of the invention, production of said antibody will also increase (Crouse et al., Mol Cell Biol 3: 257, 1983). Alternative methods of expressing the gene of the invention exist and are known to the person of skills in the art. For example, a modified zinc finger protein can be engineered that is capable of binding the expression regulatory elements upstream of the gene of the invention; expression of the said engineered zinc finger protein (ZFN) in the host cell of the invention leads to increases in protein production (see e.g. Reik et al., Biotechnol. Bioeng., 97(5): 1180-1189, 2006). Moreover, ZFN can stimulate the integration of a DNA into a predetermined genomic location, resulting in high-efficiency site-specific gene addition (Moehle et al, Proc Natl Acad Sci USA, 104: 3055, 2007).

The antibody of interest (e.g., an anti-VSIG4 antibody) may be prepared by growing a culture of the transformed host cells under culture conditions necessary to express the desired antibody. The resulting expressed antibody may then be purified from the culture medium or cell extracts. Soluble forms of the antibody of interest (e.g., an anti-VSIG4 antibody) can be recovered from the culture supernatant. It may then be purified by any method known in the art for purification of an immunoglobulin molecule, for example, by chromatography (e.g., ion exchange, affinity, particularly by Protein A affinity for Fc, and so on), centrifugation, differential solubility or by any other standard technique for the purification of proteins. Suitable methods of purification will be apparent to a person of ordinary skills in the art.

Another aspect of the invention thus relates to a method for the production of an antibody (e.g., an anti-VSIG4 antibody) described herein, said method comprising the steps of:
a) growing the above-described host cell in a culture medium under suitable culture conditions; and
b) recovering the antibody (e.g., an anti-VSIG4 antibody), from the culture medium or from said cultured cells.

The antibody obtained by culturing the transformant can be used in a non-purified state. Impurities can be removed by additional various commons methods like centrifuge or ultrafiltration, and the resultant may be subjected to dialysis, salt precipitation, chromatography or the like, in which the method may be used either singly or in combination thereof. Among them, affinity chromatography is most widely used, including ion exchange chromatography, size exclusion chromatography, hydrophobic interaction chromatography, hydroxyapatite chromatography, and the like.

Pharmaceutical Compositions

In another aspect, the present disclosure provides compositions comprising an anti-VSIG4 antibody or an antigen-binding fragment thereof, such as e.g., any of the anti-VSIG4 antibodies described herein, or a conjugate thereof, i.e., an immunoconjugate comprising one of the anti-VSIG4 antibodies described herein.

These compositions are particularly useful for e.g. stimulating an immune response in a subject. The antibody of the present invention which specifically binds to VSIG4 induces T cell activation by binding to VSIG4 protein, which inhibits T cell activation, and thus the antibody can stimulate an immune response.

The compositions described herein are also useful for treating cancer. A protective anti-tumour immunity can be established by administration of such compositions comprising the anti-VSIG4 antibody, antigen-binding fragments thereof, or conjugates thereof, which are disclosed herein.

Optionally, the compositions can comprise one or more additional therapeutic agents, such as the immune checkpoint inhibitors described below. The compositions will usually be supplied as part of a sterile, pharmaceutical composition that will normally include a pharmaceutically acceptable carrier and/or excipient. In another aspect, the invention thus provides a pharmaceutical composition comprising the anti-VSIG4 antibody or conjugate thereof, and a pharmaceutically acceptable carrier and/or an excipient.

This composition can be in any suitable form (depending upon the desired method of administering it to a patient). The compositions utilised in the methods described herein can be administered, for example, intravitreally (e.g., by intravitreal injection), by eye drop, intramuscularly, intravenously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumourally, peritoneally, subcutaneously, subconjunctivally, intravesicularly, mucosally, intrapericardially, intraumbilically, intraocularly, intraorbitally, orally, topically, transdermally, by inhalation, by injection, by implantation, by infusion, by continuous infusion, by localised perfusion bathing target cells directly, by catheter, by lavage, in cremes, or in lipid compositions. The compositions utilised in the methods described herein can also be administered systemically or locally. The method of administration can vary depending on various factors (e.g., the compound or composition being administered and the severity of the condition, disease, or disorder being treated). The most suitable route for administration in any given case will depend on the particular antibody, the subject, and the nature and severity of the disease and the physical condition of the subject. The anti-VSIG4 antibody, an antigen-binding fragment thereof, or its conjugate can be formulated as an aqueous solution and administered by subcutaneous injection.

Pharmaceutical compositions can be conveniently presented in unit dose forms containing a predetermined amount of an anti-VSIG4, an antigen-binding fragment thereof, or a conjugate thereof per dose. Such a unit can contain for example but without Limitation 5 mg to 5 g, for example 10 mg to 1 g, or 20 to 50 mg. Pharmaceutically acceptable carriers for use in the disclosure can take a wide variety of forms depending, e.g., on the condition to be treated or route of administration.

Pharmaceutical compositions of the disclosure can be prepared for storage as lyophilised formulations or aqueous solutions by mixing the antibody having the desired degree of purity with optional pharmaceutically-acceptable carriers, excipients or stabilisers typically employed in the art (all of which are referred to herein as “carriers”), i.e., buffering agents, stabilising agents, preservatives, isotonifiers, non-ionic detergents, antioxidants, and other miscellaneous additives. See, Remington's Pharmaceutical Sciences, 16th edition (Osol, ed. 1980). Such additives must be nontoxic to the recipients at the dosages and concentrations employed.

Buffering agents help to maintain the pH in the range which approximates physiological conditions. They can be present at concentration ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium gluconate mixture, gluconic acid-sodium hydroxide mixture, gluconic acid-potassium gluconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives can be added to retard microbial growth, and can be added in amounts ranging from 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilisers” can be added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilisers refer to a broad category of excipients which can range in function from a bulking agent to an additive which solubilises the therapeutic agent (i.e., an anti-VSIG4 antibody, an antigen-binding fragment thereof, or a conjugate thereof) or helps to prevent denaturation or adherence to the container wall. Typical stabilisers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, α-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trisaccacharides such as raffinose; and polysaccharides such as dextran. Stabilisers can be present in the range from 0.1 to 10,000 weights per part of weight active protein (e.g., an anti-VSIG4 antibody or a conjugate comprising such an antibody).

Non-ionic surfactants or detergents (also known as “wetting agents”) can be added to help solubilise the anti-VSIG4 antibody (or the conjugate thereof) as well as to protect the therapeutic protein against agitation-induced aggregation, which also permits the formulation to be exposed to shear surface stressed without causing denaturation of the protein. Suitable non-ionic surfactants include polysorbates (20, 80, etc.), polyoxamers (184, 188, etc.), pluronic polyols, polyoxyethylene sorbitan monoethers (TWEEN®-20, TWEEN®-80, etc.). Non-ionic surfactants can be present in a range of about 0.05 mg/ml to about 1.0 mg/ml, for example about 0.07 mg/ml to about 0.2 mg/ml.

Additional miscellaneous excipients include bulking agents (e.g., starch), chelating agents (e.g., EDTA), antioxidants (e.g., ascorbic acid, methionine, vitamin E), and cosolvents.

The present disclosure is further directed to a pharmaceutical composition comprising at least:
i) an anti-VSIG4 antibody, an antigen-binding fragment thereof, or a conjugate thereof, as disclosed herein; and
ii) a second therapeutic agent, for example an immune checkpoint inhibitor as described below,
as combination products for simultaneous, separate, or sequential use.

“Simultaneous use” as used herein refers to the administration of the two compounds of the composition according to the invention in a single and identical pharmaceutical form.

“Separate use” as used herein refers to the administration, at the same time, of the two compounds of the composition according to the invention in distinct pharmaceutical forms.

“Sequential use” as used herein refers to the successive administration of the two compounds of the composition according to the invention, each in a distinct pharmaceutical form.

Compositions of anti-VSIG4 antibodies (or antigen-binding fragments thereof or conjugates thereof) and second therapeutic agents, such as e.g., immune checkpoint inhibitors, can be administered singly, as mixtures of one or more anti-VSIG4 antibodies (or antigen-binding fragments thereof or conjugates thereof) and/or one or more a second therapeutic agent (for example an immune checkpoint inhibitor as described below), in mixture or combination with other agents useful for treating cancer or adjunctive to other therapy for cancer. Examples of suitable combination and adjunctive therapies are provided below.

Encompassed by the present disclosure are pharmaceutical kits containing anti-VSIG4 antibodies (or antigen-binding fragments thereof or conjugates thereof) and described herein. The pharmaceutical kit is a package comprising an anti-VSIG4 antibody (e.g., either in lyophilised form or as an aqueous solution) and one or more of the following:
A second therapeutic agent, for example an immune checkpoint inhibitor as described below;
A device for administering the anti-VSIG4 antibody, for example a pen, needle and/or syringe; and
Pharmaceutical grade water or buffer to resuspend the antibody if the inhibitor is in antibody form.

Each unit dose of the anti-VSIG4 antibody (or antigen-binding fragments thereof or conjugates thereof) can be packaged separately, and a kit can contain one or more-unit doses (e.g., two-unit doses, three-unit doses, four-unit doses, five-unit doses, eight-unit doses, ten-unit doses, or more). In a specific embodiment, the one or more-unit doses are each housed in a syringe or pen.

Effective Amounts

The anti-VSIG4 antibodies, antigen-binding fragment thereof, and conjugates thereof, optionally in combination with immune checkpoint inhibitors, will generally be used in an amount effective to achieve the intended result, for example an amount effective to treat cancer in a subject in need thereof. Pharmaceutical compositions comprising anti-VSIG4 antibodies (or antigen-binding fragments thereof or conjugates thereof) and/or immune checkpoint inhibitors can be administered to patients (e.g., human subjects) at therapeutically effective dosages.

Determination of the effective amount is well within the capability of those skilled in the art, especially in light of the detailed disclosure provided herein. Toxicity and therapeutic efficacy of a compound or a conjugate can be determined by standard pharmaceutical procedures in cell cultures and in experimental animals. The effective amount of present combination or other therapeutic agent to be administered to a subject will depend on the stage, category and status of the disease (e.g., cancer) and characteristics of the subject, such as general health, age, sex, body weight and drug tolerance. The effective amount of the present therapeutic agent or combination to be administered will also depend on administration route and dosage form. Dosage amount and interval can be adjusted individually to provide plasma levels of the active compound that are sufficient to maintain desired therapeutic effects.

The amount of the anti-VSIG4 antibody or antigen-binding fragment thereof or conjugates thereof administered will depend on a variety of factors, including the nature and stage of the disease being treated (e.g., cancer), the form, route and site of administration, the therapeutic regimen (e.g., whether the therapeutic agent is used in combination with immune checkpoint inhibitors), the age and condition of the particular subject being treated, the sensitivity of the patient being treated with the antibodies or the conjugates. The appropriate dosage can be readily determined by a person skilled in the art. Ultimately, a physician will determine appropriate dosages to be used. This dosage can be repeated as often as appropriate. If side effects develop the amount and/or frequency of the dosage can be altered or reduced, in accordance with normal clinical practice. The proper dosage and treatment regimen can be established by monitoring the progress of therapy using conventional techniques known to the people skilled of the art.

Effective dosages can be estimated initially from in vitro assays. For example, an initial dose for use in animals may be formulated to achieve a circulating blood or serum concentration of anti-VSIG4 antibody that is at or above the binding affinity of the antibody for VSIG4 as measured in vitro. Calculating dosages to achieve such circulating blood or serum concentrations taking into account the bioavailability of the particular antibody is well within the capabilities of skilled artisans. For guidance, the reader is referred to Fingl & Woodbury, “General Principles” in Goodman and Gilman's The Pharmaceutical Basis of Therapeutics, Chapter 1, latest edition, Pagamonon Press, and the references cited therein. Initial dosages can be estimated from in vivo data, such as animal models. Animal models useful for testing the efficacy of compounds to treat particular diseases such as cancer are generally well known in the art. Ordinarily skilled artisans can routinely adapt such information to determine dosages suitable for human administration.

The effective dose of the anti-VSIG4 antibody as described herein can range from about 0.001 to about 75 mg/kg per single (e.g., bolus) administration, multiple administrations or continuous administration, or to achieve a serum concentration of 0.01-5000 μg/ml serum concentration per single (e.g., bolus) administration, multiple administrations or continuous administration, or any effective range or value therein depending on the condition being treated, the route of administration and the age, weight and condition of the subject. In a certain embodiment, each dose can range from about 0.5 μg to about 50 μg per kilogram of body weight, for example from about 3 μg to about 30 μg per kilogram body weight.

Amount, frequency, and duration of administration will depend on a variety of factors, such as the patient's age, weight, and disease condition. A therapeutic regimen for administration can continue for 2 weeks to indefinitely, for 2 weeks to 6 months, from 3 months to 5 years, from 6 months to 1 or 2 years, from 8 months to 18 months, or the like. Optionally, the therapeutic regimen provides for repeated administration, e.g., once daily, twice daily, every two days, three days, five days, one week, two weeks, or one month. The repeated administration can be at the same dose or at a different dose. The administration can be repeated once, twice, three times, four times, five times, six times, seven times, eight times, nine times, ten times, or more. A therapeutically effective amount of anti-VSIG4 antibody or a conjugate thereof (optionally in combination with immune checkpoint inhibitors) can be administered as a single dose or over the course of a therapeutic regimen, e.g., over the course of a week, two weeks, three weeks, one month, three months, six months, one year, or longer.

Methods of Treatment

The ability of the present anti-VSIG4 antibodies to induce an immune response, e.g., by promoting macrophage polarisation, notably polarisation of TAMs, and/or antagonising VSIG4 anti-inflammatory function and/or inhibiting VSIG4-mediated immunosuppression, makes them useful for treating a variety of conditions mediated by VSIG4, including cancer. Therapeutic intervention on the VSIG4 inhibitory pathway thus represents a promising approach to modulate inflammation and T cell-mediated immunity for the treatment of a wide variety of cancers.

The anti-VSIG4 antibody, an antigen-binding fragment thereof, or conjugate, described herein may thus be used in methods for treating cancer, induce the release of pro-inflammatory cytokines by macrophages, induce CD4⁺ T cell proliferation, induce CD8⁺ T cell proliferation, induce CD4⁺ T cell cytokine production, and induce CD8⁺ T cell cytokine production, wherein said methods comprise administering an effective amount of an anti-VSIG4 antibody, an antigen-binding fragment thereof, or a conjugate to a patient in need thereof. Preferably, the anti-VSIG4 antibody, an antigen-binding fragment thereof, or conjugate, described herein may thus be used in methods for treating cancer, wherein the treatment comprises inducing the release of pro-inflammatory cytokines by macrophages, inducing CD4⁺ T cell proliferation, inducing CD8⁺ T cell proliferation, inducing CD4⁺ T cell cytokine production, and/or inducing CD8⁺ T cell cytokine production, and wherein said methods comprise administering an effective amount of an anti-VSIG4 antibody, an antigen-binding fragment thereof, or a conjugate to a patient in need thereof. More preferably, the anti-VSIG4 antibody, an antigen-binding fragment thereof, or conjugate, described herein may thus be used in methods for treating cancer, wherein the treatment comprises switching the polarisation of macrophages from an M2 to an M1 phenotype, and wherein said methods comprise administering an effective amount of an anti-VSIG4 antibody, an antigen-binding fragment thereof, or a conjugate to a patient in need thereof. Even more preferably, said macrophages are TAMs, and switching the polarisation of these TAMs towards an M1 phenotype leads to a reduction of immunosuppressive and anti-inflammatory activities. The therapeutic methods described herein may comprise administration of the antibodies biding specifically VSIG4 described herein, or antigen-binding fragments thereof, or conjugates comprising these antibodies as disclosed herein, to a patient in need thereof. The VSIG4 antibodies, antigen-binding fragments, and conjugates thereof, disclosed herein, are thus useful in regulating immunity, especially T cell immunity, for the treatment of cancer.

Accordingly, an aspect of the present disclosure relates to an anti-VSIG4 antibody or antigen-biding fragment thereof or conjugate thereof for use in the treatment of a cancer in a patient. Also provided herein is a method of treating cancer in a patient in need thereof, said method comprising the administration of an anti-VSIG4 antibody, an antigen-binding fragment thereof, or a conjugate disclosed herein to the patient. The present disclosure also relates to the use of an anti-VSIG4 antibody or antigen-biding fragment thereof or conjugate thereof for making a medicament for treating a cancer.

In an embodiment, the disclosure relates to a composition comprising an anti-VSIG4 antibody disclosed herein, or an antigen-biding fragment or a conjugate thereof, for use in the treatment of a cancer in a patient. Also provided herein is a method of treating cancer in a patient in need thereof, said method comprising the administration of a composition comprising an anti-VSIG4 antibody disclosed herein, or an antigen-biding fragment or a conjugate thereof, to the patient. The present disclosure also relates to the use of a composition comprising an anti-VSIG4 antibody disclosed herein, or an antigen-biding fragment or a conjugate thereof, for making a medicament for treating a cancer.

In some embodiments, the cancer is selected from a bladder cancer, breast cancer, cervical cancer, colon cancer, endometrial cancer, oesophageal cancer, fallopian tube cancer, gall bladder cancer, gastrointestinal cancer, head-and-neck cancer, haematological cancer (e.g., leukaemia, lymphomas, or myelomas), laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma, and prostate cancer.

The present antibodies are particularly useful because they can induce an immune response in a patient, e.g., a cancer patient, as detailed above. Thus, in an embodiment, the anti-VSIG4 antibody or antigen-biding fragment thereof or conjugate thereof is for use in the treatment of a cancer in a patient, wherein the use comprises inducing an immune response in the patient. Also provided herein is a method of treating cancer in a patient in need thereof, the method comprising administering an anti-VSIG4 antibody, an antigen-binding fragment thereof, or a conjugate disclosed herein to the patient and inducing an immune response in this patient. The present disclosure also relates to the use of an anti-VSIG4 antibody or antigen-biding fragment thereof or conjugate thereof for making a medicament for treating a cancer, wherein the treatment comprises inducing an immune response in the patient.

In an embodiment, the disclosure relates to a composition comprising an anti-VSIG4 antibody disclosed herein, or an antigen-biding fragment or a conjugate thereof, for use in the treatment of a cancer in a patient, wherein the use comprises inducing an immune response in the patient. Also provided herein is a method of treating cancer in a patient in need thereof, the method comprising administering an anti-VSIG4 antibody, an antigen-binding fragment thereof, or a conjugate disclosed herein to the patient and inducing an immune response in this patient. The present disclosure also relates to the use of a composition comprising an anti-VSIG4 antibody disclosed herein, or an antigen-biding fragment or a conjugate thereof, for making a medicament for treating a cancer, wherein the treatment comprises inducing an immune response in the patient.

An embodiment provides an anti-VSIG4 antibody or antigen-biding fragment thereof or conjugate thereof for use in inducing an immune response in a cancer patient. Also provided herein is a method of inducing an immune response in a cancer patient in need thereof, said method comprising the administration of an anti-VSIG4 antibody, an antigen-binding fragment thereof, or a conjugate disclosed herein to the patient. The present disclosure also relates to the use of an anti-VSIG4 antibody or antigen-biding fragment thereof or conjugate thereof for making a medicament for inducing an immune response in a cancer patient.

In an embodiment, the disclosure relates to a composition comprising an anti-VSIG4 antibody disclosed herein, or an antigen-biding fragment or a conjugate thereof, for use in inducing an immune response in a cancer patient. Also provided herein is a method of an immune response in a cancer patient in need thereof, said method comprising the administration of a composition comprising an anti-VSIG4 antibody disclosed herein, or an antigen-biding fragment or a conjugate thereof, to the patient. The present disclosure also relates to the use of a composition comprising an anti-VSIG4 antibody disclosed herein, or an antigen-biding fragment or a conjugate thereof, for making a medicament for inducing an immune response in a cancer patient.

The immune response thus generated by the antibody disclosed herein includes, without Limitation, polarisation of macrophages, notably TAM, to an M1 phenotype, induction of pro-inflammatory cytokines release by macrophages, induction of CD4⁺ T cell proliferation, induction of CD8⁺ T cell proliferation, induction of CD4⁺ T cell cytokine production, and induction of CD8⁺ T cell cytokine production. Preferably, switching the polarisation of TAMs towards an M1 phenotype leads to a reduction of immunosuppressive activity, angiogenesis and tumour promotion.

The anti-VSIG4 antibody, or antigen-binding fragment or conjugate thereof, may be admixed with a second therapeutic agent.

A “therapeutic agent” encompasses biological agents, such as an antibody, a peptide, a protein, an enzyme, and chemotherapeutic agents. The therapeutic agent also encompasses immuno-conjugates of cell-binding agents (CBAs) and chemical compounds, such as antibody-drug conjugates (ADCs). The drug in the conjugates can be a cytotoxic agent, such as one described herein.

As used herein, the anti-VSIG4 antibody, or antigen-binding fragment or conjugate thereof, and the other therapeutic agent are said to be administered successively if they are administered to the patient on the same day, for example during the same patient visit. Successive administration can occur 1, 2, 3, 4, 5, 6, 7 or 8 hours apart. In contrast, the anti-VSIG4 antibody, or antigen-binding fragment or conjugate thereof, of the disclosure and the other therapeutic agent are said to be administered separately if they are administered to the patient on the different days, for example, the anti-VSIG4 antibody, or antigen-binding fragment or conjugate thereof, of the disclosure and the other therapeutic agent can be administered at a 1-day, 2-day or 3-day, one-week, 2-week or monthly intervals. In the methods of the present disclosure, administration of the anti-VSIG4 antibody, or antigen-binding fragment or conjugate thereof, of the disclosure can precede or follow administration of the other therapeutic agent.

As a non-limiting example, the anti-VSIG4 antibody, or antigen-binding fragment or conjugate thereof, and other therapeutic agent can be administered concurrently for a period of time, followed by a second period of time in which the administration of the anti-VSIG4 antibody, or antigen-binding fragment or conjugate thereof, of the disclosure and the other therapeutic agent is alternated.

Combination therapies of the present disclosure can result in a greater than additive, or a synergistic, effect, providing therapeutic benefits where neither the anti-VSIG4 antibody, or antigen-binding fragment or conjugate thereof, nor the other therapeutic agent is administered in an amount that is, alone, therapeutically effective. Thus, such agents can be administered in lower amounts, reducing the possibility and/or severity of adverse effects.

In a preferred embodiment, the other therapeutic agent is a chemotherapeutic agent. Said chemotherapeutic agent is preferably an alkylating agent, an anti-metabolite, an anti-tumour antibiotic, a mitotic inhibitor, a chromatin function inhibitor, an anti-angiogenesis agent, an anti-oestrogen, an anti-androgen or an immunomodulator.

The term “alkylating agent,” as used herein, refers to any substance which can cross-link or alkylate any molecule, preferably nucleic acid (e.g., DNA), within a cell. Examples of alkylating agents include nitrogen mustard such as mechlorethamine, chlorambucol, melphalen, chlorydrate, pipobromen, prednimustin, disodic-phosphate or estramustine; oxazophorins such as cyclophosphamide, altretamine, trofosfamide, sulfofosfamide or ifosfamide; aziridines or imine-ethylenes such as thiotepa, triethylenamine or altetramine; nitrosourea such as carmustine, streptozocin, fotemustin or lomustine; alkyle-sulfonates such as busulfan, treosulfan or improsulfan; triazenes such as dacarbazine; or platinum complexes such as cis-platinum, oxaliplatin and carboplatin.

The expression “anti-metabolites,” as used herein, refers to substances that block cell growth and/or metabolism by interfering with certain activities, usually DNA synthesis. Examples of anti-metabolites include methotrexate, 5-fluoruracil, floxuridine, 5-fluorodeoxyuridine, capecitabine, cytarabine, fludarabine, cytosine arabinoside, 6-mercaptopurine (6-MP), 6-thioguanine (6-TG), chlorodesoxyadenosine, 5-azacytidine, gemcitabine, cladribine, deoxycoformycin and pentostatin.

As used herein, “anti-tumour antibiotics” are compounds which may prevent or inhibit DNA, RNA and/or protein synthesis. Examples of anti-tumour antibiotics include doxorubicin, daunorubicin, idarubicin, valrubicin, mitoxantrone, dactinomycin, mithramycin, plicamycin, mitomycin C, bleomycin, and procarbazine.

“Mitotic inhibitors,” as used herein, prevent normal progression of the cell cycle and mitosis. In general, microtubule inhibitors or taxoids such as paclitaxel and docetaxel are capable of inhibiting mitosis. Vinca alkaloid such as vinblastine, vincristine, vindesine and vinorelbine are also capable of inhibiting mitosis.

As used herein, the terms “chromatin function inhibitors” or “topoisomerase inhibitors” refer to substances which inhibit the normal function of chromatin modelling proteins such as topoisomerase I or topoisomerase II. Examples of chromatin function inhibitors include, for topoisomerase I, camptothecine and its derivatives such as topotecan or irinotecan, and, for topoisomerase II, etoposide, etoposide phosphate and teniposide.

As used herein, the term “anti-angiogenesis agent” refers to any drug, compound, substance or agent which inhibits growth of blood vessels. Exemplary anti-angiogenesis agents include, but are by no means limited to, razoxin, marimastat, batimastat, prinomastat, tanomastat, ilomastat, CGS-27023A, halofuginon, COL-3, neovastat, BMS-275291, thalidomide, CDC 501, DMXAA, L-651582, squalamine, endostatin, SU5416, SU6668, interferon-alpha, EMD121974, interleukin-12, IM862, angiostatin and vitaxin.

As used herein, the terms “anti-oestrogen” or “anti-estrogenic agent” refer to any substance which reduces, antagonizes or inhibits the action of oestrogen. Examples of anti-oestrogen agents are tamoxifen, toremifene, raloxifene, droloxifene, iodoxyfene, anastrozole, letrozole, and exemestane.

As used herein, the terms “anti-androgens” or “anti-androgen agents” refer to any substance which reduces, antagonises or inhibits the action of an androgen. Examples of anti-androgens are flutamide, nilutamide, bicalutamide, sprironolactone, cyproterone acetate, finasteride and cimitidine.

“Immunomodulators” as used herein are substances which stimulate the immune system.

Examples of immunomodulators include interferon, interleukin such as aldesleukine, OCT-43, denileukin diflitox and interleukin-2, tumoural necrose fators such as tasonermine or others immunomodulators such as lentinan, sizofiran, roquinimex, pidotimod, pegademase, thymopentine, poly I:C or levamisole in conjunction with 5-fluorouracil.

For more detail, the person of skill in the art can refer to the manual edited by the “Association Françaíse des Enseignants de Chimie Thérapeutique” and entitled “Traité de chimie thérapeutique”, vol. 6, Médicaments antitumouraux et perspectives dans le traitement des cancers, edition TEC & DOC, 2003.

It can also be mentioned as chemical agents or cytotoxic agents, all kinase inhibitors such as, for example, gefitinib or erlotinib.

More generally, examples of suitable chemotherapeutic agents include but are not limited to 1-dehydrotestosterone, 5-fluorouracil decarbazine, 6-mercaptopurine, 6-thioguanine, actinomycin D, adriamycin, aldesleukin, alkylating agents, allopurinol sodium, altretamine, amifostine, anastrozole, anthramycin (AMC)), anti-mitotic agents, cis-dichlorodiamine platinum (II) (DDP) cisplatin), diamino dichloro platinum, anthracyclines, antibiotics, antimetabolites, asparaginase, BCG live (intravesical), betamethasone sodium phosphate and betamethasone acetate, bicalutamide, bleomycin sulfate, busulfan, calcium leucouorin, calicheamicin, capecitabine, carboplatin, lomustine (CCNU), carmustine (BSNU), Chlorambucil, Cisplatin, Cladribine, Colchicin, conjugated estrogens, Cyclophosphamide, Cyclothosphamide, Cytarabine, Cytarabine, cytochalasin B, Cytoxan, Dacarbazine, Dactinomycin, dactinomycin (formerly actinomycin), daunirubicin HCL, daunorucbicin citrate, denileukin diftitox, Dexrazoxane, Dibromomannitol, dihydroxy anthracin dione, Docetaxel, dolasetron mesylate, doxorubicin HCL, dronabinol, E. coli L-asparaginase, emetine, epoetin-α, Erwinia L-asparaginase, esterified estrogens, estradiol, estramustine phosphate sodium, ethidium bromide, ethinyl estradiol, etidronate, etoposide citrororum factor, etoposide phosphate, filgrastim, floxuridine, fluconazole, fludarabine phosphate, fluorouracil, flutamide, folinic acid, gemcitabine HCL, glucocorticoids, goserelin acetate, gramicidin D, granisetron HCL, hydroxyurea, idarubicin HCL, ifosfamide, interferon α-2b, irinotecan HCL, letrozole, leucovorin calcium, leuprolide acetate, levamisole HCL, lidocaine, lomustine, maytansinoid, mechlorethamine HCL, medroxyprogesterone acetate, megestrol acetate, melphalan HCL, mercaptipurine, mesna, methotrexate, methyltestosterone, mithramycin, mitomycin C, mitotane, mitoxantrone, nilutamide, octreotide acetate, ondansetron HCL, oxaliplatin, paclitaxel, pamidronate disodium, pentostatin, pilocarpine HCL, plimycin, polifeprosan 20 with carmustine implant, porfimer sodium, procaine, procarbazine HCL, propranolol, rituximab, sargramostim, streptozotocin, tamoxifen, taxol, tegafur, teniposide, tenoposide, testolactone, tetracaine, thioepa chlorambucil, thioguanine, thiotepa, topotecan HCL, toremifene citrate, trastuzumab, tretinoin, valrubicin, vinblastine sulfate, vincristine sulfate, and vinorelbine tartrate.

The anti-VSIG4 antibody, or antigen-binding fragment or conjugate thereof, disclosed herein can be administered to a patient in need of treatment for cancer receiving a combination of chemotherapeutic agents. Exemplary combinations of chemotherapeutic agents include 5-fluorouracil (5FU) in combination with leucovorin (folinic acid or LV); capecitabine, in combination with uracil (UFT) and leucovorin; tegafur in combination with uracil (UFT) and leucovorin; oxaliplatin in combination with 5FU, or in combination with capecitabine; irinotecan in combination with capecitabine, mitomycin C in combination with 5FU, irinotecan or capecitabine. Use of other combinations of chemotherapeutic agents disclosed herein is also possible.

The anti-VSIG4 antibody, or antigen-binding fragment or conjugate thereof, can also be combined with other therapeutic antibodies. Accordingly, therapy based on the anti-VSIG4 antibody, or antigen-binding fragment or conjugate thereof, disclosed herein can be combined with, or administered adjunctive to a different monoclonal antibody such as, for example, but not by way of Limitation, an anti-EGFR (EGF receptor) monoclonal antibody or an anti-VEGF monoclonal antibody. Specific examples of anti-EGFR antibodies include cetuximab and panitumumab. A specific example of an anti-VEGF antibody is bevacizumab.

Notably, the therapeutic methods described herein may comprise the administration of an immune checkpoint inhibitor along with the anti-VSIG4 antibody, or antigen-binding fragment or conjugate thereof. The immune checkpoint inhibitor and the anti-VSIG4 antibody, or antigen-binding fragment or conjugate thereof may be administered simultaneously, separately, or sequentially.

As used herein, a “checkpoint inhibitor” refers to a molecule, such as e.g., a small molecule, a soluble receptor, or an antibody, which targets an immune checkpoint and blocks the function of said immune checkpoint. More specifically, a “checkpoint inhibitor” as used herein is a molecule, such as e.g., a small molecule, a soluble receptor, or an antibody, that blocks certain proteins made by some types of immune system cells, such as T cells, and some cancer cells.

In a first embodiment, the immune checkpoint inhibitor is an inhibitor of any one of CTLA-4, PDL1, PDL2, PD1, B7-H3, B7-H4, BTLA, HVEM, TIGIT, TIM3, GAL9, LAG3, PSG-L1, VISTA, KIR, 2B4 (belongs to the CD2 family of molecules and is expressed on all NK, γδ, and memory CD8+(αβ) T cells), CD160 (also referred to as BY55), CGEN-15049, CHK 1 and CHK2 kinases, IDO1, A2aR and any of the various B-7 family ligands.

Exemplary immune checkpoint inhibitors include anti-CTLA-4 antibody (e.g., ipilimumab), anti-LAG-3 antibody (e.g., BMS-986016), anti-B7-H3 antibody, anti-B7-H4 antibody, anti-Tim3 antibody (e.g., TSR-022, MBG453), anti-BTLA antibody, anti-KIR antibody, anti-A2aR antibody, anti CD200 antibody, anti-PD-1 antibody (e.g., pembrolizumab, nivolumab, cemiplimab, pidilizumab), anti-PD-L1 antibody (e.g., atezolizumab, avelumab, durvalumab, BMS 936559), anti-TIGIT antibody (e.g., tiragolumab, vibostolimab), anti-VISTA antibody (e.g., JNJ 61610588), anti-CD28 antibody, anti-CD80 or -CD86 antibody, anti-B7RP1 antibody, anti-B7-H3 antibody, anti-HVEM antibody, anti-CD137 antibody (e.g., urelumab), anti-CD137L antibody, anti-OX40 (e.g., 9B12, PF-04518600, MED16469), anti-OX40L antibody, anti-CD40 or -CD40L antibody, anti-GAL9 antibody, anti-IL-10 antibody, fusion protein of the extracellular domain of a PD-1 ligand, e.g. PDL-1 or PD-L2, and IgG1 (e.g., AMP-224), fusion protein of the extracellular domain of a OX40 ligand, e.g. OX40L, and IgG1 (e.g., MED16383), IDO1 drug (e.g., epacadostat) and A2aR drug. A number of immune checkpoint inhibitors have been approved or are currently in clinical trials. Such inhibitors include ipilimumab, pembrolizumab, nivolumab, cemiplimab, pidilizumab, atezolizumab, avelumab, durvalumab, tiragolumab, vibostolimab, BMS 936559, JNJ 61610588, urelumab, 9B12, PF-04518600, BMS-986016, TSR-022, MBG453, MED16469, MED16383, and epacadostat.

Examples of immune checkpoints inhibitors are listed for example in Marin-Acevedo et al., Journal of Hematology & Oncology 11: 8, 2018; Kavecansky and Pavlick, AJHO 13(2): 9-20, 2017; Wei et al., Cancer Discov 8(9): 1069-86, 2018.

Preferably, the immune checkpoint inhibitor is an inhibitor of CTLA-4, LAG-3, Tim3, PD-1, PD-L1, PSG-L1, VISTA, CD137, OX40, or IDO1.

Methods of Diagnosis

VSIG4 is overexpressed in a variety of cancers, indicating that VSIG4 is dependable biomarker for diagnosing a cancer. Reagents such as the Labelled antibodies provided herein, which bind to VSIG4 protein, can thus be used for diagnostic purposes to detect, diagnose, or monitor a cell proliferative disease, disorder or condition such as e.g., cancer.

Anti-VSIG4 antibodies provided herein can be used to detect VSIG4 or assay VSIG4 levels in a biological sample using classical immunohistological methods as described herein or as known to those of skill in the art (e.g., see Jalkanen et al., 1985, J. Cell. Biol. 101:976-985; and Jalkanen et al., 1987, J. Cell. Biol. 105:3087-3096). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin.

Thus, in a first aspect, the invention relates to an in vitro method for detecting a VSIG4-expressing cancer in a subject, said method comprising the steps of:
a) contacting a biological sample of said subject with anti-VSIG4 antibody disclosed herein, or antigen-binding fragment thereof; and
b) detecting the binding of said antibody, or antigen-binding fragment thereof, with said biological sample.

According to the present method, the binding of the anti-VSIG4 antibody indicates the presence of a VSIG4-expressing cancer. Preferably, the binding of the anti-VSIG4 antibody in immune infiltrates of the tumour microenvironment indicates the presence of a VSIG4-expressing cancer.

The invention also relates to an in vitro method for detecting a VSIG4-expressing cancer in a subject, said method comprising the steps of:
a) contacting a biological sample of said subject with an anti-VSIG4 antibody, or an antigen-binding fragment thereof; and
b) quantifying the binding of said antibody, or antigen-binding fragment thereof, with said biological sample.

According to the present method, the binding of the anti-VSIG4 antibody indicates the presence of a VSIG4-expressing cancer. Preferably, the binding of the anti-VSIG4 antibody in immune infiltrates of the tumour microenvironment indicates the presence of a VSIG4-expressing cancer.

As will be apparent to the skilled artisan, the level of antibody binding to VSIG4 may be quantified by any means known to the person of skills in the art, as detailed hereafter. Preferred methods include the use of immunoenzymatic assays, such as ELISA or ELISPOT, immunofluorescence, immunohistochemistry (IHC), radio-immunoassay (RIA), or FACS.

The quantification of step b) of the present method is a direct reflection of the level of VSIG4 expression in the sample, notably in immune infiltrates of the tumour microenvironment. The present method thus allows for identifying a VSIG4-expressing cancer by determining the level of expression of VSIG4, as described above. In a preferred embodiment, the level of expression of VSIG4 in said sample, notably in immune infiltrates of the tumour microenvironment, is compared to a reference level.

According to a further preferred embodiment, the invention relates to an in vitro method for detecting a VSIG4-expressing cancer in a subject, said method comprising the steps of:
a) determining the level of expression of VSIG4 in a biological sample of said subject; and
b) comparing the level of expression of step a) with a reference level;
   wherein an increase in the assayed level of VSIG4 in step a) compared to the reference level is indicative of a VSIG4-expressing cancer.

The invention also relates to an in vitro method for diagnosing a VSIG4-expressing cancer in a subject, said method comprising the steps of:
a) determining the level of expression of VSIG4 in a biological sample of said subject; and
b) comparing the level of expression of step a) with a reference level;
   wherein an increase in the assayed level of VSIG4 in step (b) compared to the reference level is indicative of a VSIG4-expressing cancer.

The expression level of VSIG4 is advantageously compared or measured in relation to levels in a control cell or sample also referred to as a “reference level” or “reference expression level”. “Reference level”, “reference expression level”, “control level” and “control” are used interchangeably in the specification. A “control level” means a separate baseline level measured in a comparable control cell, which is generally disease or cancer free. The said control cell may be from the same individual, since, even in a cancerous patient, the tissue which is the site of the tumour still comprises non-tumour healthy tissue. It may also originate from another individual who is normal or does not present with the same disease from which the diseased or test sample is obtained. Within the context of the present invention, the term “reference level” refers to a “control level” of expression of VSIG4 used to evaluate a test level of expression of VSIG4 in a cancer cell-containing sample of a patient. For example, when the level of VSIG4 in the biological sample of a patient is higher than the reference level of VSIG4, the cells will be considered to have a high level of expression, or overexpression, of VSIG4. The reference level can be determined by a plurality of methods. Expression levels may thus define VSIG4 bearing cells or alternatively the level of expression of VSIG4 independent of the number of cells expressing VSIG4. Thus, the reference level for each patient can be prescribed by a reference ratio of VSIG4, wherein the reference ratio can be determined by any of the methods for determining the reference levels described herein.

For example, the control may be a predetermined value, which can take a variety of forms. It can be a single cut-off value, such as a median or mean. The “reference level” can be a single number, equally applicable to every patient individually, or the reference level can vary, according to specific subpopulations of patients. Thus, for example, older men might have a different reference level than younger men for the same cancer, and women might have a different reference level than men for the same cancer. Alternatively, the “reference level” can be determined by measuring the level of expression of VSIG4 in non-oncogenic cancer cells from the same tissue as the tissue of the neoplastic cells to be tested. As well, the “reference level” might be a certain ratio of VSIG4 in the neoplastic cells of a patient relative to the VSIG4 levels in non-tumour cells within the same patient. The “reference level” can also be a level of VSIG4 of in vitro cultured cells, which can be manipulated to simulate tumour cells, or can be manipulated in any other manner which yields expression levels which accurately determine the reference level. On the other hand, the “reference level” can be established based upon comparative groups, such as in groups not having elevated VSIG4 levels and groups having elevated VSIG4 levels. Another example of comparative groups would be groups having a particular disease, condition or symptoms and groups without the disease. The predetermined value can be arranged, for example, where a tested population is divided equally (or unequally) into groups, such as a low-risk group, a medium-risk group and a high-risk group.

The reference level can also be determined by comparison of the level of VSIG4 in populations of patients having the same cancer. This can be accomplished, for example, by histogram analysis, in which an entire cohort of patients is graphically presented, wherein a first axis represents the level of VSIG4, and a second axis represents the number of patients in the cohort whose tumour cells express VSIG4 at a given level. Two or more separate groups of patients can be determined by identification of subsets populations of the cohort which have the same or similar levels of VSIG4. Determination of the reference level can then be made based on a level which best distinguishes these separate groups. A reference level also can represent the levels of two or more markers, one of which is VSIG4. Two or more markers can be represented, for example, by a ratio of values for levels of each marker.

Likewise, an apparently healthy population will have a different ‘normal’ range than will have a population which is known to have a condition associated with expression of VSIG4. Accordingly, the predetermined value selected may take into account the category in which an individual falls. Appropriate ranges and categories can be selected with no more than routine experimentation by those of ordinary skill in the art. By “elevated” “increased” it is meant high relative to a selected control. Typically, the control will be based on apparently healthy normal individuals in an appropriate age bracket.

It will also be understood that the controls according to the invention may be, in addition to predetermined values, samples of materials tested in parallel with the experimental materials. Examples include tissue or cells obtained at the same time from the same subject, for example, parts of a single biopsy, or parts of a single cell sample from the subject.

Preferably, the reference level of VSIG4 is the level of expression of VSIG4 in normal tissue samples (e.g., from a patient not having a VSIG4-expressing cancer, or from the same patient before disease onset).

A more definitive diagnosis of a VSIG4-expressing cancer may allow health professionals to employ preventative measures or aggressive treatment earlier thereby preventing the development or further progression of the VSIG4-expressing cancer.

Hereinbelow, the present invention is explained in detail in view of the examples. However, the following examples are given only for exemplification of the present invention, and it is evident that the present invention is not limited to the following examples.

### EXAMPLES

### Example 1: Properties of VSIG4 Long and Short Forms

### 1-1. Expression of VSIG4 Long and Short Forms on Macrophages

VSIG4 is known to be expressed by macrophages. In order to test whether there is a difference in expression, the presence of each of the two forms of VSIG4, i.e., VSIG4(L) and VSIG4(S), were sought in extracts of M1 and M2 macrophages.

50 ng/ml of IFN-γ (285-IF, R&D) was added to GM-CSF differentiated M0-macrophages for polarisation into pro-inflammatory M1-macrophages. 20 ng/ml of each of the following cytokines: IL-4 (130.093.922, Miltenyi Biotec), IL-10 (217-IL/CF, R&D) and TGF-β (130.095.066, Miltenyi Biotec) were added to M-CSF differentiated M0-macrophages for polarisation into immunosuppressive M2-macrophages. Differentiated M0-macrophages were incubated with cytokines at 37° C., 5% CO₂ for 2 days. M1 and M2 polarised macrophages were obtained at day 8. Polarised macrophages were activated with 100 ng/ml LPS (L4516, Sigma) for 4 hours at 37° C., 5% CO₂. Macrophages were then harvested and washed in culture medium. The binding of target antibodies on polarised M1- and M2-macrophages was assessed by flow cytometry following LPS activation.

15 μg of M1 and M2 protein extracts, along with 100 ng of hVSIG4(L)-hFc and hVSIG4(S)-hFc were run on a SDS-PAGE gel, transferred to a membrane and probed with either a polyclonal antibody specific for VSIG4 (AF4646, R&D Systems, Minneapolis, MN, USA) or a goat isotype control.

Two bands of the expected sizes were seen in extracts from M2 macrophages (FIG. 1B). This result confirms previous data on the expression of VSIG4 in macrophages. Further, it shows that both hVSIG4(S) and hVSIG4(L) are expressed in macrophages.

### 1-2. Expression of VSIG4 Long and Short Forms in Tumours.

Expression of both VSIG4 forms in tumours was investigated. The VSIG4 gene is located on the X chromosome and 7 exons are depicted in the gene model. This gives rise to 2 messenger RNAs produced by alternative splicing. One long form, Long-VSIG4 (uc004dwh.2) and one short form, Short-VSIG4 (uc004dwi.2), which yield hVSIG4(L) and hVSIG4(S), respectively. The Cancer Genome Atlas (TCGA) contains data resulting from the characterisation of over 20,000 primary cancer and matched normal samples spanning 33 cancer types. TCGA tumour expression data (Tumour TCGA RNASeq) were used to determine the expression patterns of the two isoforms with ISOexpresso (Yang et al., BMC Genomics (2016) 17: 631; http://wiki.tgilab.org/ISOexpresso/).

The results are shown in Table 3.

TABLE 3
Percentage expression of Long VSIG4 Isoform
and Short VSIG4 Isoform by indications

| | % Long-VSIG4 | % Short-VSIG4 |
|---|---|---|
| Indications | (uc004dwh.2) | (uc004dwi.2) |
| Bladder Cancer | 66 | 32 |
| Breast Cancer | 74 | 25 |
| Colon Cancer | 74 | 25 |
| Head and Neck Cancer | 70 | 29 |
| Lung Adenocarcinoma | 74 | 21 |
| Lung Squamous Cell Carcinoma | 72 | 24 |
| Ovarian Cancer | 78 | 18 |
| Prostate Cancer | 72 | 24 |
| Stomach Cancer | 77 | 20 |
| Adrenocortical carcinoma | 55 | 41 |
| Pheochromocytoma and | 68 | 26 |
| Paraganglioma | | |
| Cholangiocarcinoma | 67 | 29 |
| Bladder Urothelial Carcinoma] | 66 | 32 |
| Acute Myeloid Leukemia | 34 | 0 |
| Glioblastoma multiforme | 71 | 22 |
| Breast invasive carcinoma | 74 | 25 |
| Brain Lower Grade Glioma | 71 | 18 |
| Cervical squamous cell | 60 | 35 |
| carcinoma and endocervical | | |
| adenocarcinoma | | |
| Uveal Melanoma | 65 | 33 |
| Colon adenocarcinoma | 74 | 25 |
| Uterine Corpus Endometrial | 49 | 48 |
| Carcinoma | | |
| Esophageal carcinoma | 76 | 22 |
| Head and Neck squamous | 70 | 29 |
| cell carcinoma | | |
| Kidney Chromophobe | 66 | 34 |
| Kidney renal clear | 69 | 25 |
| cell carcinoma | | |
| Kidney renal papillary | 65 | 31 |
| cell carcinoma | | |
| Liver hepatocellular carcinoma | 54 | 44 |
| Lung adenocarcinoma | 74 | 21 |
| Lung squamous cell carcinoma | 72 | 24 |
| Ovarian serous | 78 | 18 |
| cystadenocarcinoma | | |
| Pancreatic adenocarcinoma | 70 | 26 |
| Mesothelioma | 61 | 30 |
| Prostate adenocarcinoma | 72 | 24 |
| Rectum adenocarcinoma | 71 | 27 |
| Stomach adenocarcinoma | 77 | 20 |
| Testicular Germ | 71 | 27 |
| Cell Tumours | | |
| Thymoma | 51 | 45 |
| Thyroid carcinoma | 70 | 27 |
| Uterine Carcinosarcoma | 59 | 40 |

Both the long and the short VSIG4 isoforms are expressed in tumours.

1-3. Inhibition of CD4⁺ T Cells Activation bV hVSIG4(S) and hVSIG4(L)

96 well plates were coated 4 h at 37° C. with 2.5 μg/ml of anti CD3 OKT3 antibody (BioxCell ref BE0001-2 clone OKT3) in 100 μl/well, washed twice with PBS and coated with 10 μg/ml of recombinant proteins (VSIG4(L)-Fc (SEQ ID NO. 83), VGIG4(S)-Fc (SEQ ID NO. 84), PDL1-Fc (R&D Systems 156-B7) or an isotype control hIgG1 (c9G4)) and incubated overnight at 4° C. Wells were washed twice with PBS and 200,000 of CD4⁺ T cells negatively purified from healthy donor and CFSE Labelled were added to each well in 200 μl of culture medium.

After 3 days culture, the supernatants were transferred to a new plate and analysed by MSD for IFN γ release. In addition, cells were analysed by flow cytometry to assess their proliferation rate.

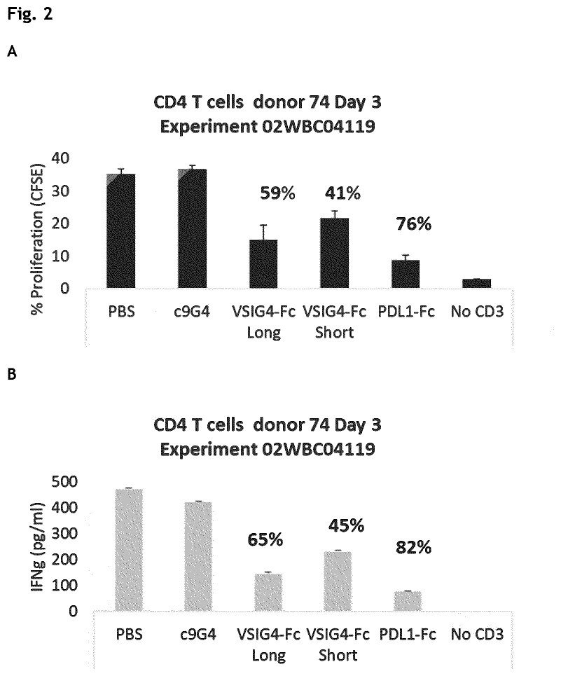

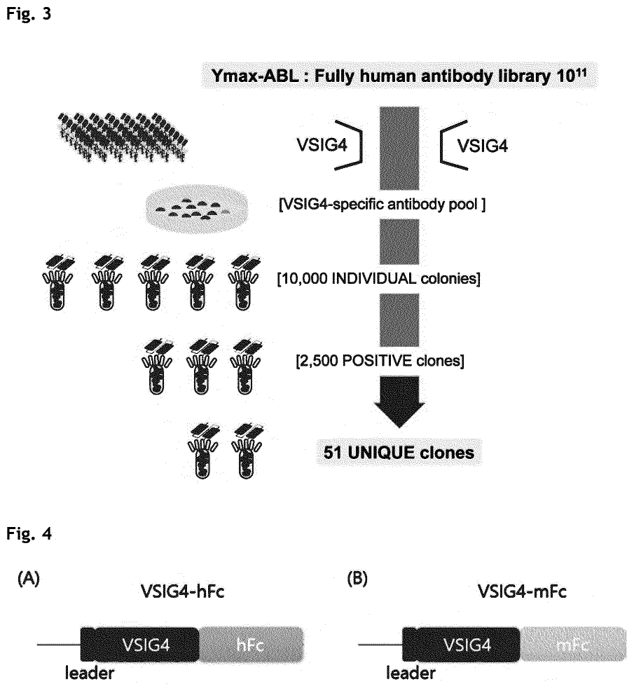

FIG. 2A shows that both forms of VSIG4 (VSIG4(S) and VSIG4(L)) inhibit the proliferation of CD4⁺ T cells. Likewise, both forms inhibit the release of IFNγ by CD4⁺ T cells (FIG. 2B). Similar results were obtained with CD8⁺ T cells (data not shown).

### Example 2. Production and Purification of VSIG4 Antigen

### 2-1. Construction of Vector for Expressing VSIG4 Antigen Protein

For cloning the VSIG4 protein, amplification was carried out with Jurkat cell cDNA library (Stratagene, USA) by polymerase chain reaction (PCR) using primers for VSIG4 (Table 4), which include restriction enzyme sites Sfi I at 5′ and 3′ for obtaining only the extracellular domain (20Arg-Ser281). The amplified PCR product was fused at the carboxy terminal with human Fc (hFc) or mouse Fc (mFc) by using N293F vector (FIG. 4).

TABLE 4
PCR Primers for VSIG4 cloning

| Primer | Sequence Information |
|---|---|
| Name | (5′→3′) |
| VSIG4-F | cgtcccatcctggaagtgccag |
| | (SEQ ID NO: 51) |
| VSIG4-R | gctctttcctggcccagcactgg |
| | (SEQ ID NO: 52) |

### 2-2. Expression and Purification of VSIG4 Antigen Protein

By using PEI (polyethylenimine: #23966, Polysciences, USA), HEK293F cells (Invitrogen, USA) were transfected with the prepared VSIG4 antigen plasmid. Thereafter, the cells were cultured for 7 days in FreeStyle 293 Expression Medium (#AG100009, Thermo Fisher Scientific, USA), which is a serum-free medium. The cell culture containing the VSIG4 antigen was collected and centrifuged for 10 minutes at 5,000 rpm, and the residual cells and floating materials were removed by using a 0.22 μm TOP-filter (Millipore, USA). Based on affinity chromatography using protein A agarose resin, the first purification of the antigen was carried out. The protein obtained after the first purification was subjected to the second purification using Superdex 200 (1.5 cm×100 cm) gel filtration chromatography.

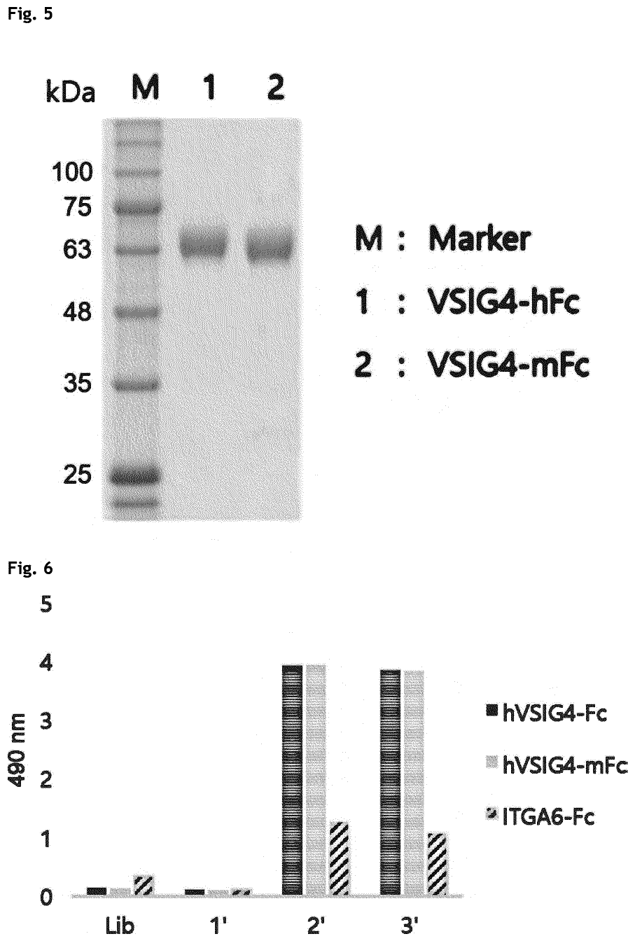

Purity of the purified protein was determined by SDS-PAGE (sodium dodecyl sulfate polyacrylamide gel electrophoresis) at reducing conditions. As a result, as it is shown in FIG. 5, the purity of the purified VSIG4-hFc and VSIG4-mFc protein was found to be 95% or higher.

### Example 3. Selection of Human Anti-VSIG4 Antibody

### 3-1. Biopanning

VSIG4-hFc and VSIG4-mFc prepared in Example 2, and VSIG4-his (12163-H08H) protein antigen, which has been purchased from Sino Biological Inc., and ITGA6-Fc used as an indicator of non-specific binding were coated (50 μg) on an immunosorb tube followed by blocking.

With regard to the human antibody library phage, E. coli cells were infected with human scFv (single-chain variable fragment) library having 2.7×10¹⁰ variety, and then cultured for 16 hours at 30° C. After the culture, centrifugation was carried out to concentrate the supernatant with PEG (polyethylene glycol, Sigma), and the resultant was dissolved in PBS buffer to prepare a human antibody library. The library phage was added to the immunosorb tube and allowed to react therewith for 2 hours at room temperature. Then, after washing with 1×PBS-Tween20 (PBS-T) and 1×PBS, only the scFv-phages specifically bound to the antigen were eluted.

Through the panning process in which E. coli cells were infected again with the eluted phages for amplification, a pool of positive phage was obtained. With the phage amplified in the first round, the second and third round pannings were carried out in the same manner as the first round except that the number of PBS-T washing step was increased. As a result, as it is shown in Table 5, it was confirmed that the number of phages bound to the antigen during the third-round panning has slightly increased in terms of the output relative to input.

TABLE 5
Comparison of antibody titre according to panning

| Number of | Number of | Number of |
|---|---|---|
| panning | input phages | output phages |
| 1 | 3 × 10¹¹ | 1.05 × 10⁶ |
| 2 | 5 × 10¹² | 2.5 × 10⁵ |
| 3 | 3 × 10¹⁰ | 2.4 × 10⁷ |

### 3-2. Polyphage ELISA

In order to examine the antigen-specificity of the positive poly scFv-phage antibody pool, which has been obtained from the panning process of each round of Example 3-1, polyphage ELISA (enzyme linked immunoassay) was carried out.

The cell stock frozen after each panning of the first to third round was added to a medium containing 2× YTCM (yeast extract 10 g, tryptone 17 g, NaCl 5 g, chloramphenicol 34 μg/ml), 2% glucose, and 5 mM magnesium chloride (MgCl₂) (OD600=0.1), and then cultured for 2 to 3 hours at 37° C. (OD600=0.5 to 0.7). Then, after infection with M1 helper phage, culture for 16 hours at 30° C. in a medium containing 2× YTCMK (2× YTCM, kanamycin 35 μg/ml), 5 mM magnesium chloride and 1 mM IPTG was carried out. The cultured cells were centrifuged (4,500 rpm, 15 minutes, 4° C.) and the supernatant was transferred to a new tube. On a 96-well immuno-plate (#439454, NUNC, USA), each of the two antigens was coated, in an amount of 100 ng per well, at 4° C. for 16 hours using a coating buffer, and then each well was blocked by using 4% skim milk dissolved in PBS. After that, each well was washed with 0.2 ml of PBS-T, and the first- to third-panning poly scFv-phage was added to each well, each in an amount of 100 μl, followed by reaction for 2 hours at room temperature. Then again, each well was washed 4 times with 0.2 ml of PBS-T, and, after diluting anti-M13-HRP (Amersham 27-9421-01) as a secondary antibody at 1:2,000, the reaction with an antibody was carried out for 1 hour at room temperature. After washing with PBS-T, OPD tablet (Sigma. 8787-TAB) was prepared in PC buffer (0.1 M Na₂HPO₄, 0.005 M Na-Citrate, pH 5.0) and added to the well (100 μl per well) to have colour development for 10 minutes. Then, the absorbance at 490 nm was measured by using a spectrophotometer (Molecular Device, USA).

The results are shown in FIG. 6, wherein it was confirmed by ELISA that the binding affinity of the antibodies for the two VSIG4 antigens was enriched in the second and third poly scFv-phage pools.

### 3-3. Selection of Positive Phage

The colonies obtained from the multiclone phage antibody group having high binding property (third panning) were cultured for 16 hours at 37° C. in a 96-deep well plate (#90030, Bioneer, Korea) by using 1 ml medium containing 2× YTCM, 2% glucose, and 5 mM magnesium chloride. From the cultured cells, 100 to 200 μl were collected (OD600=0.1), and then added to a medium containing 1 ml 2× YTCM, 2% glucose and 5 mM magnesium chloride and cultured for 2 to 3 hours at 37° C. in a 96-deep well plate (OD600=0.5 to 0.7). Infection of M1 helper phage was carried out to have MOI value of 1:20, and then cultured for 16 hours at 30° C. in a medium containing 2× YTCMK, 5 mM magnesium chloride, and 1 mM IPTG.

On a 96-well immuno-plate, the antigen VSIG4 was coated, in an amount of 100 ng per well, at 4° C. for 16 hours, and then each well was blocked by using 4% skim milk dissolved in PBS. After that, each well was washed with 0.2 ml of PBS-T, and the single clone scFv-phage (100 scFv-phages, respectively) cultured for 16 hours was added to each well in an amount of 100 μl to have a reaction for 2 hours at room temperature. Then again, each well was washed 4 times with 0.2 ml of PBS-T, and, after diluting anti-M13-HRP as a secondary antibody at 1:2,000, the reaction with the antibody was carried out for 1 hour at room temperature. After washing with PBS-T (0.2 ml), colour development was allowed to occur and the absorbance at 490 nm was measured.

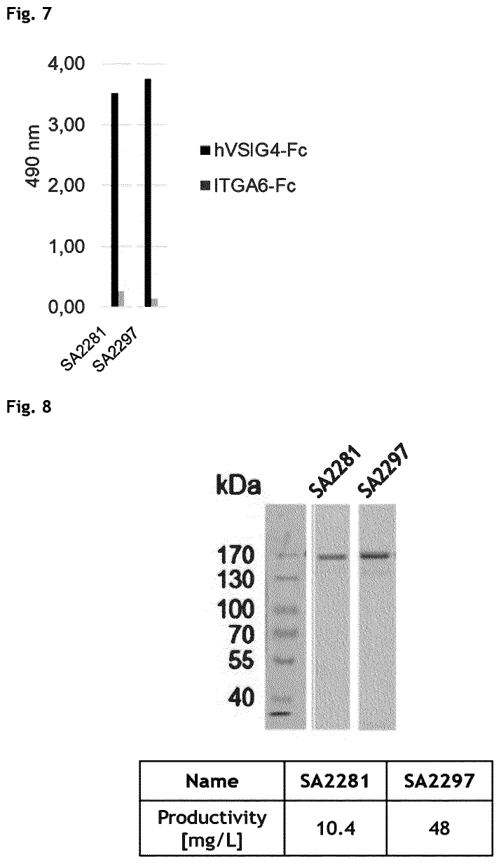

As a result, dozens of mono-phage clones having high binding affinity were obtained and two of them are shown in FIG. 7.

### 3-4. Nucleotide Sequencing of Positive Phage Antibody

For the monoclones which had been selected as described in 3-3, DNA-prep was carried out by using a kit for DNA purification (Qiagen, Germany) to obtain DNA. Macrogen, Korea was requested to carry out the sequencing of the DNA. In view of the sequencing result, CDR site of heavy chain variable region (VH) and light chain variable region (VL) of the selected antibody was determined. Then, the similarity between those antibodies and germ line antibody group was examined by using Ig BLAST program provided in NCBI webpage (http://www.ncbi.nlm.nih.gov/igblast/). As a result, dozens of VSIG4-specific phage antibodies were obtained, and three (3) of them were characterised more specifically. They are summarised in Table 2.

### Example 4. Production of Human Anti-VSIG4 Antibody

4-1. Conversion of scFv Form into IgG Form

In order to convert the selected anti-VSIG4 monoclonal phage antibodies into full IgG form, the DNA sequences corresponding to the variable regions of heavy chain and light chain were subjected to PCR (iCycler iQ, BIO-RAD, USA) by using primers comprising restriction enzyme sites for SfiI/NheI and SfiI/BglII, respectively. The heavy chain and light chain PCR products were digested together with each expression vector having a corresponding restriction enzyme site, and the resulting DNA products were purified with DNA-gel extraction kit (Qiagen). For ligation, the vector DNA (1 μl, 10 ng), the heavy chain or light chain DNA (100 to 200 ng, 15 μl), 10× buffer (2 μl), ligase (1 U/μl, 1 μl), and water were admixed with one another, kept for 1 to 2 hours at room temperature, and added to E. coli cells for transformation (competent cell, XL1-blue). The resultant was kept on ice for 5 minutes, and then applied with heat shock at 42° C. for 90 seconds. After the heat shock, the cells were added with 1 ml of medium and cultured for 1 hour at 37° C. followed by spreading on an LB Amp plate and culture for 16 hours at 37° C. Thus-obtained colonies were collected and inoculated with 5 ml of an LB Amp medium. After culture for 16 hours at 37° C., DNA-prep was carried out by using DNA-prep kit (Nuclogen). DNA sequencing of the thus-obtained DNA was requested (Macrogen, Korea).

As a result, it was confirmed that each of the heavy chain and light chain of the antibody clones for VSIG4, which have been converted into full IgG, corresponds to the sequence of phage antibody. After that, the heavy chain and light chain plasmid DNA with identified sequence was used for antibody production.

### 4-2. Production of Human Antibody

The prepared expression vectors containing a heavy chain and a light chain, respectively, were subjected to co-transfection in HEK-293F cells at a ratio of 6:4. Seven days after the co-transfection, the supernatant was collected and subjected to centrifuging and filtering through a 0.22 μm Top-filter to remove floating materials. The resulting supernatant was subjected to protein A affinity chromatography to purify the IgG antibody. After the purification, the antibody was separated using glycine buffer, and buffer exchange was carried out such that the final resuspension buffer was PBS. The purified antibody was quantified by BCA and Nano-drop to determine the production amount. The antibody was then subjected to SDS-PAGE analysis with a load of 5 μg for each of reducing condition and non-reducing condition. Accordingly, the purity and mobility of the purified protein were determined.

As shown in FIG. 8, the human anti-VSIG4 monoclonal antibodies SA2281 and SA2297 were detected at a size of at least 150 kDa under non-reducing condition. The productivity was 10.4 mg/L for SA2281 and 48 mg/L for SA2297.

### Example 5. VSIG4 Binding Properties of Human Anti-VSIG4 Antibody

5-1. Antibody Binding Specificity for VSIG4 on Cell Surface

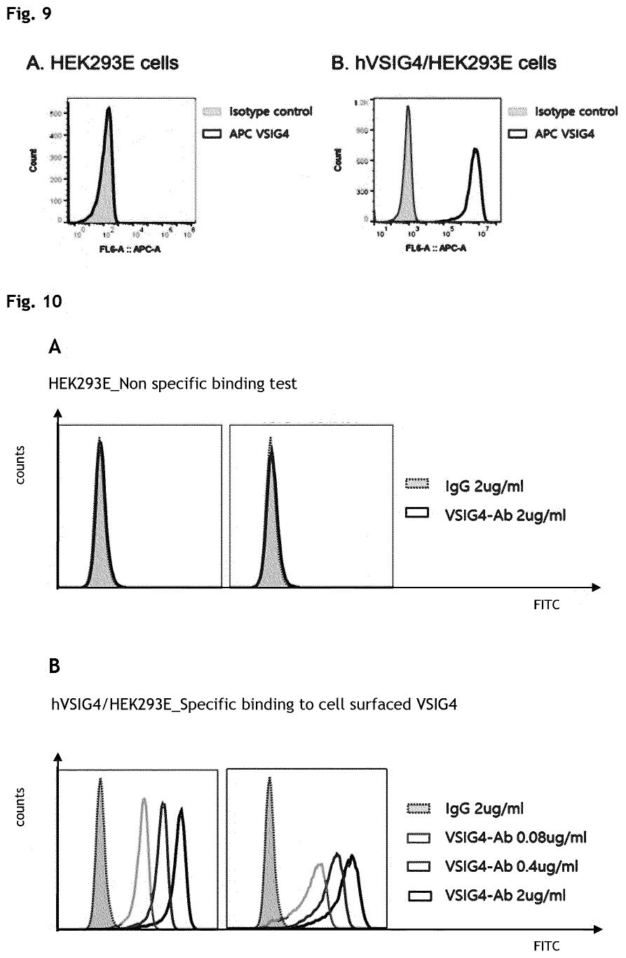

In order to secure a transformed cell pool in which human VSIG4 is overexpressed, HEK293E cell was transfected with pcDNA3.1 plasmid comprising human VSIG4, and then a selection process was carried out in a selection medium containing 400 μg/ml Zeocin (#R25001, Thermo Fisher Scientific). After the selection process, the HEK293E cell pool in which human VSIG4 is overexpressed was obtained by determining the expression state by FACS (fluorescence activated cell sorting) analysis using anti-human VSIG4 antibody conjugated with APC (allophycocyanin) fluorescent material (#17-5757-42, ebioscience, USA) (FIG. 9B). On the other hand, as to the parent HEK293E cells, it was confirmed by the same FACS analysis that there is no basal expression of VSIG4 (FIG. 9A). The analysis of antibody property was carried out for both anti-human VSIG4 antibodies SA2281 and SA2297 by using those two types of cells.

In order to confirm the cell binding by the anti-VSIG4 antibody, 0.5×10⁶ cells were prepared for each sample and allowed to react with the antibody at 0.08 μg/ml, 0.4 μg/ml, or 2 μg/ml for 30 minutes at 4° C. Thereafter, the cells were washed 3 times with a buffer containing 2% PBS, and, after the reaction for 20 minutes at 4° C. with anti-human IgG antibody (#FI-3000, Vectorlabs) linked with FITC (fluorescein isothiocyanate) fluorescent material, the cells were washed by the same washing process as above, followed by suspension in 0.5 ml PBS containing 2% FPS. The cells were then analysed by FACS on a flow cytometer. As a result, it was found that both SA2281 (left panel) and SA2297 (right panel) bind well to the human VSIG4-overexpressing cells in a concentration dependent manner (FIG. 10B). However, there was no binding in HEK293E cells having no basal expression of VSIG4 (FIG. 10A).

This result indicates that both antibodies specifically bind to the human VSIG4 antigen.

5-2. Binding of Human Anti-VSIG4 Antibody to the Human Native VSIG4

The binding properties of the anti-VSIG4 antibodies were evaluated by FACS analyses on HEK293E cell expressing human VSIG4 using increasing antibody concentrations. The same experiment was performed with m6H8, a murine monoclonal antibody recognising VSIG4, and its humanised version hz6H8-A2, described in WO 2020/069507. For that purpose, cells (1×10⁶ cells/ml) were incubated with either SA2281, SA2297, m6H8 or hz6H8-A2 for 20 minutes at 4° C. in FACS buffer (PBS, 0.1% BSA, 0.01% NaN₃). They were then washed 3 times and incubated with the appropriate secondary antibody coupled with Alexa 488 for 20 additional minutes at 4° C. in the dark before being washed 3 times in FACS buffer. The binding of the anti-VSIG4 antibodies, m6H8 or hz6H8-A2 was immediately performed on viable cells which were identified using propidium iodide (that stains dead cells). The maximum of signal intensity obtained with each antibody was designed as Bₘₐₓ and expressed in mean of fluorescence intensity (MFI). The EC₅₀ of binding expressed in molarity (M) was calculated using a nonlinear regression analysis (GraphPad Prims 4.0).

The titration curve of each murine or chimeric Ab demonstrated that all generated antibodies are capable of recognising the native VSIG4 form with a typical saturation profile. The binding EC₅₀ of each antibody was determined using a non-linear regression analysis. EC₅₀ values are summarised in Table 6.

TABLE 6
Binding of the anti-VSIG4 antibodies to huVSIG4.

| Mab | EC₅₀ (M) |
|---|---|
| SA2281 | 4.605E−10 |
| SA2297 | 1.181E−09 |
| m6H8 | 2.283E−09 |
| hz6H8-A2 | 8.917E−10 |

5-3. Binding of Human Anti-VSIG4 Antibody to Long and Short Forms of VSIG4

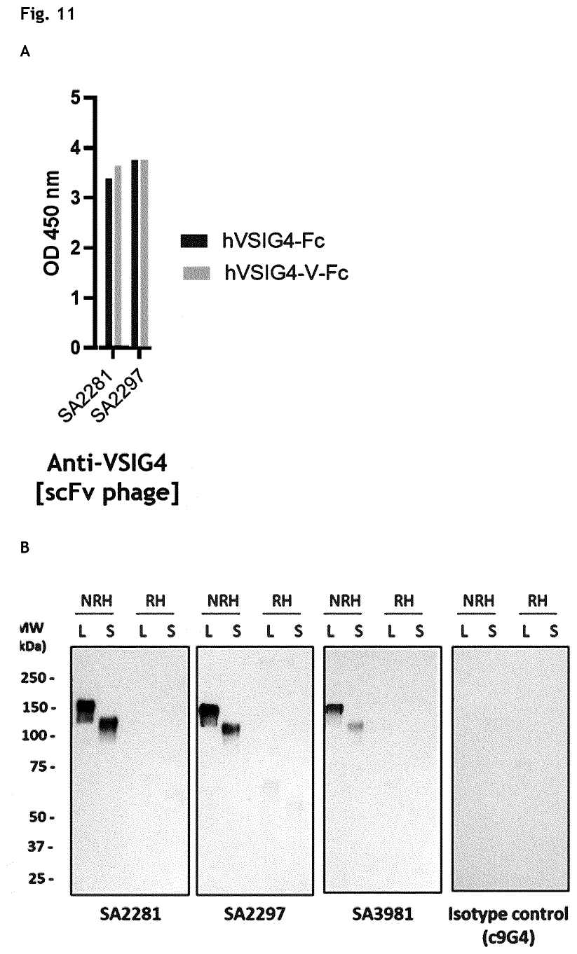

In a first series of experiments, the binding of each of the anti-VSIG4 antibodies SA2281 and SA2297 to each of the long and the short forms of VSIG4 was tested by ELISA. Specific binding of each of the anti-VSIG4 antibodies tested to both forms was detected under these conditions (FIG. 11A).

In order to confirm this result, the binding of the anti-VSIG4 antibodies to each of the long and the short forms of VSIG4 was assayed by western blotting. In each case, 100 ng of hVSIG4 long-hFc (L) and hVSIG4 short-hFc (S) were probed with the specific anti-VSIG4 antibody.

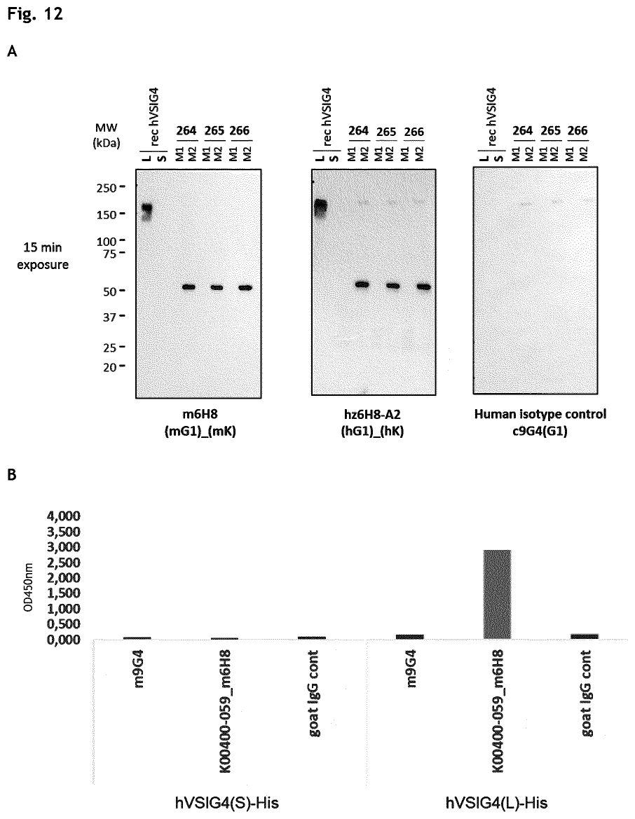

As shown in FIG. 11B, two bands of the expected size were observed for each of SA2281 and SA2297. By contrast, m6H8, a murine monoclonal antibody recognising VSIG4, and its humanised version hz6H8-A2 described in WO 2020/069507, only bind the long form of VSIG4 (FIG. 12A). This result was confirmed in an ELISA assay (FIG. 12B).

5-4. Epitope Mapping of the Anti-VSIG4 Antibodies

In order to delineate the epitope recognised by SA2281 and SA2297, their ability to bind a series of soluble VSIG4 protein carrying specific mutations was assayed by ELISA. The constructs used are detailed in Table 7.

TABLE 7
Constructions used for epitope mapping.

| | | | PRO- | WHOLE | | |
|---|---|---|---|---|---|---|
| | | DNA | TEIN | CLONE | | |
| | | (SEQ | (SEQ | (SEQ | | |
| | | ID | ID | ID | STRUC- | MUTA- |
| NAME | GENE | NO.) | NO.) | NO.) | TURE | TION |
| hVSIG4- | VSIG4 | SEQ | SEQ | SEQ | [LS]- | |
| Fc | | ID | ID | ID | [VSIG4]- | |
| | | NO. | NO. | NO. | AS- | |
| | | 53 | 54 | 55 | [FC] | |
| hVSIG4- | VSIG4 | SEQ | SEQ | SEQ | [LS]- | |
| V- | | ID | ID | ID | [VSIG4]- | |
| Fc | | NO. | NO. | NO. | AS- | |
| | | 56 | 57 | 58 | [FC] | |
| VSIG4- | VSIG4 | SEQ | SEQ | SEQ | [LS]- | E24T, |
| M1- | | ID | ID | ID | QGAVGA- | V25A, |
| Fc | | NO. | NO. | NO. | [VSIG4]- | E27H, |
| | | 59 | 60 | 61 | LAASAAS- | V29L, |
| | | | | | [FC] | T30A |
| VSIG4- | VSIG4 | SEQ | SEQ | SEQ | [LS]- | D36T, |
| M2- | | ID | ID | ID | QGAVGA- | N38R, |
| Fc | | NO. | NO. | NO. | [VSIG4]- | L39M, |
| | | 62 | 63 | 64 | LAASAAS- | T42S |
| | | | | | [FC] | |
| VSIG4- | VSIG4 | SEQ | SEQ | SEQ | [LS]- | Q59L, |
| M3- | | ID | ID | ID | QGAVGA- | G61N, |
| Fc | | NO. | NO. | NO. | [VSIG4]- | S62Y, |
| | | 65 | 66 | 67 | LAASAAS- | D63H, |
| | | | | | [FC] | V65A |
| VSIG4- | VSIG4 | SEQ | SEQ | SEQ | [LS]- | 177V, |
| M4- | | ID | ID | ID | QGAVGA- | A80T, |
| Fc | | NO. | NO. | NO. | [VSIG4]- | Y82F, |
| | | 68 | 69 | 70 | LAASAAS- | Q83R |
| | | | | | [FC] | |
| VSIG4- | VSIG4 | SEQ | SEQ | SEQ | [LS]- | H87E, |
| M5- | | ID | ID | ID | QGAVGA- | H90R, |
| Fc | | NO. | NO. | NO. | [VSIG4]- | K91Q, |
| | | 71 | 72 | 73 | LAASAAS- | V92P |
| | | | | | [FC] | |
| VSIG4- | VSIG4 | SEQ | SEQ | SEQ | [LS]- | S97A, |
| M6- | | ID | ID | ID | QGAVGA- | Q99T, |
| Fc | | NO. | NO. | NO. | [VSIG4]- | S101N, |
| | | 74 | 75 | 76 | LAASAAS- | T102P |
| | | | | | [FC] | |
| VSIG4- | VSIG4 | SEQ | SEQ | SEQ | [LS]- | R108Q, |
| M7- | | ID | ID | ID | QGAVGA- | S109G, |
| Fc | | NO. | NO. | NO. | [VSIG4]- | H110Y, |
| | | 77 | 78 | 79 | LAASAAS- | T112V, |
| | | | | | [FC] | E114A |
| VSIG4- | VSIG4 | SEQ | SEQ | SEQ | [LS]- | T119D, |
| M8- | | ID | ID | ID | QGAVGA- | P120Q, |
| Fc | | NO. | NO. | NO. | [VSIG4]- | D121A, |
| | | 80 | 81 | 82 | LAASAAS- | N123H, |
| | | | | | [FC] | Q124L, |
| | | | | | | V1251 |

Note:
hVSIG4-Fc is the long VSIG4 form.
hVSIG4-V-Fc is the short VSIG4 form.
All mutations were made in the short form.
LS: Leader sequence
FC: Human lgG1 Fc domain, including hinge SEQ ID No. 85)

On a 96-well immuno-plate, the various antigens were coated, in an amount of 100 ng per well, at 4° C. for 16 hours, and then each well was blocked by using 4% skim milk dissolved in PBS. After that, each well was washed with 0.2 ml of PBS-T, and each of the scFv-phages cultured for 16 hours was added to each well in an amount of 100 μl to have a reaction for 2 hours at room temperature. Then again, each well was washed 4 times with 0.2 ml of PBS-T, and, after diluting anti-M13-HRP as a secondary antibody at 1:2,000, the reaction with the antibody was carried out for 1 hour at room temperature. After washing with PBS-T (0.2 ml), colour development was allowed to occur and the absorbance at 490 nm was measured.

The assay showed that SA2281 bound all constructs, except VSIG4-M7-Fc, for which no significant binding was detected. The epitope recognised by the antibody SA2281 comprises therefore at least one of the amino acids R108, S109, H110, T112, and E114.

Binding was only abolished for SA2297 when either of VSIG4-M7-Fc and VSIG4-M8-Fc were used. On the other hand, this antibody was capable of binding all the other constructs, as observed in the ELISA assay. The epitope recognised by the antibody SA2297 comprises at least one of the amino acids R108, S109, H110, T112, and E114, and at least one of the amino acids T119, P120, D121, N123, Q124, and V125.

SA3981, another antibody isolated in the screening process described above, was also tested. No signal was detected in the ELISA assay when the antibody was incubated with VSIG4-M7-Fc, whereas binding was observed with all the other fusion proteins. Thus SA3981 binds an epitope which comprises at least one of the amino acids R108, S109, H110, T112, and E114.

### Example 6: Inhibition of VSIG4 Binding of C3b and iC3b

One of VSIG4 functions is to be a complement receptor on macrophages, promoting phagocytosis by binding to C3b and iC3b coated particles (Small, 2016).

The ability of SA2281 and SA2297 to block these interactions was evaluated using HTRF (Homogeneous Time Resolved Fluorescence) assays. In addition, SA3981, which binds the same VSIG4 epitope as SA2281 (M7), hz6H8-A2 (described in WO 2020/069507), and SA2282, were also evaluated in these assays. Finally, the assays also included a positive control (polyclonal anti-VSIG4 antibody AF4646) and a negative control (isotype antibody c9G4).

Antibodies were tested in a dose-response assay starting at 500 nM with 5-time dilution steps. In each well of a 384-well, low volume white (non-binding surface (NBS) plates, 5 μl of compound to be tested at a 4× concentration or buffer (PBS) was mixed with 5 μl of C3b-d2 (K00400006-MID1-154) (200 nM) or iC3b (K00400006-BBC1-165)(200 nM), 5 μl of human His-tagged VSIG4 short isoform (K00400002-BR1-117) (40 nM) or PBS, and 5 μl anti 6His-Tb cryptate gold (Cisbio 61HT12TLB) (1/100). After a quick centrifugation and a 10 s plate shaking, the plate was incubated 2 h at RT, before being read on multimode plate reader adapted for TR-FRET technology (ex: 320 nm, emission 620 nm and 650 nm). Each assay was replicated in triplicate.

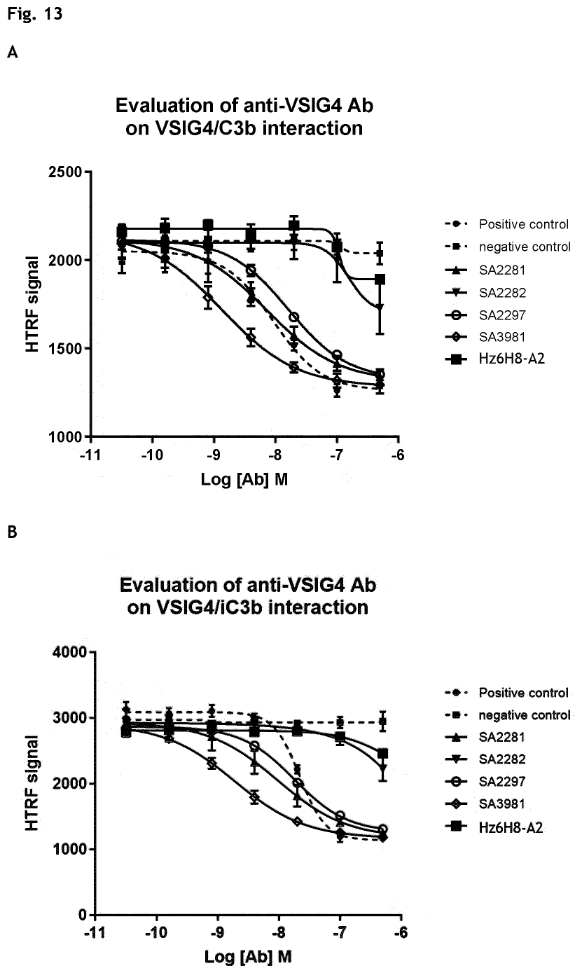

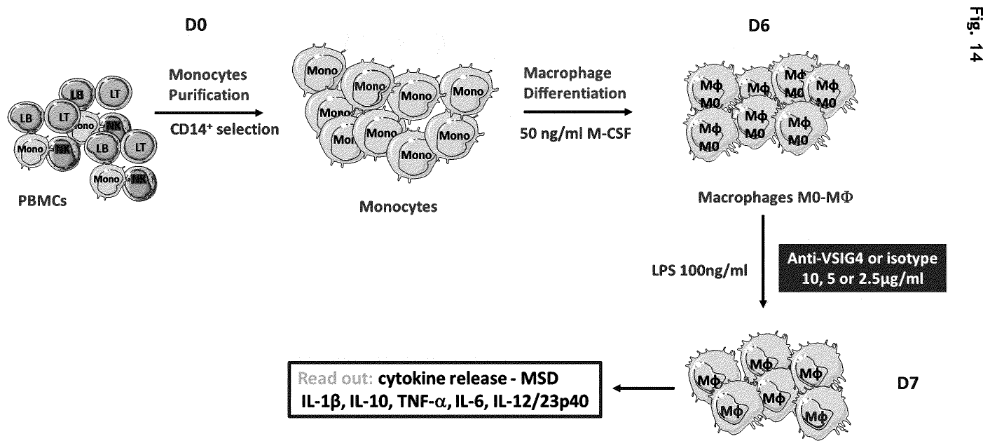

The results are shown in FIGS. 13A and 13B. The IC₅₀ are shown in Table 8 and are expressed in nM.

Similar results were obtained in either the VSIG4 C3b or iC3b interaction assays. The SA2281, SA2297 and SA3981 antibodies inhibited both VSIG4/C3b and VSIG4/iC3b interaction in a dose-dependent manner. On the other hand, neither hz6H8-A2 nor SA2282 showed any inhibition of the interaction excepted at the highest tested dose.

TABLE 8
Inhibition of VSIG4/C3b and VSIG4/iC3b
by the anti-VSIG4 antibodies.

IC₅₀ (nM)

| | C3b | iC3b |
|---|---|---|
| AF4646 | 9.2 | 21 |
| C9G4 | Nd | Nd |
| SA2281 | 7.3 | 9.5 |
| SA2282 | Nd | Nd |
| SA2297 | 16.4 | 17.9 |
| SA3981 | 1.4 | 1.9 |
| hz6H8-A2 | nd | nd |

### Example 7: Inhibition of VSIG4 Anti-Inflammatory and Immunosuppressive Functions by the Human Anti-VSIG4 Antibody

### 7-1. Inflammatory Assay

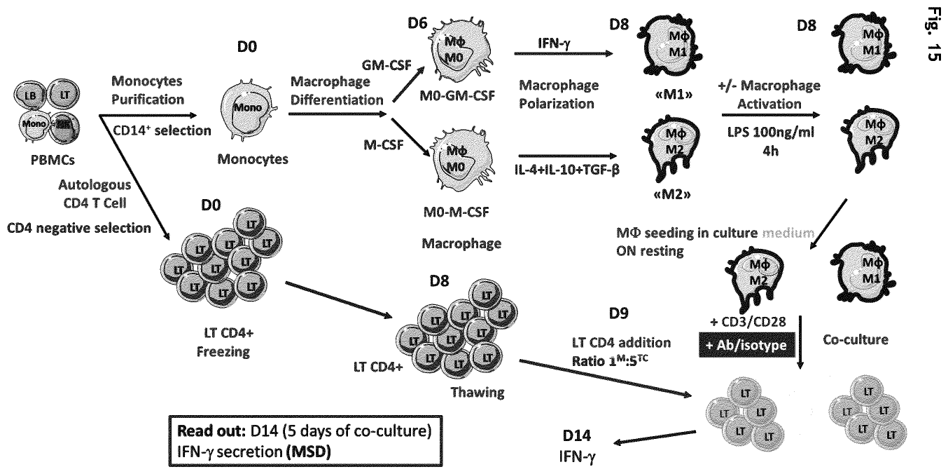

In order to assess the ability of the anti-VSIG4 antibodies to modulate the inflammatory phenotype of macrophages, a cytokine release assay was performed on macrophages treated with each of the full Ig, human anti-VSIG4 antibodies SA2281 and SA2297. The antibody SA3981 was included as a control. The experimental scheme is shown in FIG. 14.

Peripheral Blood Mononuclear Cells (PBMC) were isolated from human blood by density gradient centrifugation from cytapheresis ring provided by EFS (Etablissement Françaís du Sang). Monocytes were then purified from PBMC by positive immunomagnetic cell selection according to the manufacturer's instructions (130-050-201, Miltenyi Biotec).

Fresh monocytes were seeded in 96-well flat-bottom treated culture plates (353072, Falcon) in culture medium (RPMI 1640 medium+1% Penicillin streptomycin+1% Sodium Pyruvate+1% L-Glutamine+10% Fetal Calf Serum) containing 50 ng/ml M-CSF (130-096-492, Miltenyi Biotec). They were incubated at 37° C., 5% CO₂ for 6 days for differentiation into macrophages. Differentiated M0-macrophages were obtained at day 6.

The binding of target antibodies on differentiated M0-macrophages was assessed by flow cytometry at day 6. LPS (L4516, Sigma) was added to differentiated M0-macrophages at a final concentration of 100 ng/ml. Test antibodies or corresponding isotypes were added to differentiated M0-macrophages at three concentrations (2.5 μg/ml, 5 μg/ml and 10 μg/ml). As a control, a control antibody (R&D, Ref MAB2078, clone 287219, mIgG2a) known to simulate the release of cytokines from M0 macrophages towards a pro-inflammatory phenotype, was used at the final concentration of 5 μg/ml.

Differentiated M0-macrophages were incubated with LPS and test antibodies for 24 hours at 37° C., 5% CO₂. Cell culture supernatants were harvested at day 7 and transferred into new V-bottom 96-well plates for cytokine analysis. The concentrations of IL-10, IL-6, IL-1B, IL-12/23p40 and TNF-α were measured. The quantification was performed using the Meso Scale Discovery technology according to the manufacturer's instructions (K15UQK-4 and K151AOH-4, Meso Scale Discovery).

At least 5 donors were evaluated to take into account the heterogeneity between healthy donors. Each experimental condition was performed in triplicate and in one experiment.

The results of the assay are shown in Table 9.

TABLE 9
Modulation of cytokine release from human monocyte-derived
macrophages in response to VSIG4 antibody treatment.

| | Donor showing a modulation of cytokine release compared to isotype | | | | | |
|---|---|---|---|---|---|---|
| | TNF-α | IL-1β | IL-12p70 | IL-10 | IL-6 | Summary |
| control | D348, D349, | D344, D348, | D353 | D345, D348, | | 4 cytokines |
| Ab | D353, D354 | D349, D353 | | D349, D354 | | |
| | 4D/6 | 4D/6 | 1D/6 | 4D/6 | 0D/6 | |
| SA2281 | D348, D349, | D344, D345, | D344, D345, | D345, D349, | D344, D345, | 5 cytokines |
| | D353 | D348, D349, | D348, D349, | D354 | D348, D349, | |
| | | D353, D354 | D353, D354 | | D353, D354 | |
| | 3D/6 | 6D/6 | 6D/6 | 3D/6 | 6D/6 | |
| SA2297 | D348, D349, | D344, D345, | D344, D345, | D345, D348, | D344, D345, | 5 cytokines |
| | D353 | D348, D349, | D348, D349, | D349, D354 | D348, D349, | |
| | | D353, D354 | D353, D354 | | D353, D354 | |
| | 3D/6 | 6D/6 | 6D/6 | 4D/6 | 6D/6 | |
| SA3981 | | | D348 | | | 1 cytokine |
| | | | (10 μg/ml) | | | |
| | 0D/6 | 0D/6 | 1D/6 | 0D/6 | 0D/6 | |

Both anti-VSIG4 antibodies SA2281 and SA2297 lead to increased release of proinflammatory cytokines and/or a decrease of anti-inflammatory cytokines secretion by the macrophages, whereas no change in cytokine secretion is observed with SA3981, even though it binds the same epitope as SA2281 (i.e., M7).

SA2281 and SA2297 are thus specifically capable of modulating the phenotype of human macrophages.

### 7-2. Immunosuppressive Assay

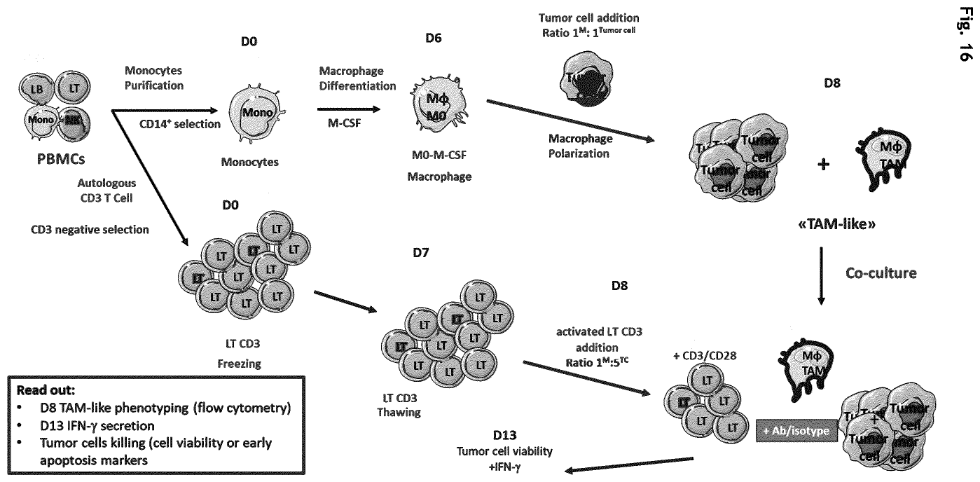

The anti-VSIG4 antibodies SA2281 and SA2297 were tested for their potential to antagonise M2-mediated T cell suppression. The immunosuppression assay is based on i) the co-culture of autologous monocyte-derived M2 macrophages and activated CD4⁺ T cells and on ii) the quantitation of IFN-γ levels as surrogate of T cell activation. The experimental scheme is shown in FIG. 15.

Peripheral Blood Mononuclear Cells (PBMC) were isolated from human blood by density gradient centrifugation from cytapheresis ring provided by EFS (Etablissement Françaís du Sang). Monocytes and CD4⁺ T cells were then purified from PBMC from the same donor: Monocytes were purified by positive immunomagnetic cell selection according to the manufacturer's instructions (130-050-201, Miltenyi Biotec), whilst CD4⁺ T cells were isolated from the non-positive fraction of monocytes purification by negative immunomagnetic cell selection according to the manufacturer's instructions (19052, STEMCELL Technologies). CD4⁺ T cells were frozen at 15×10⁶ cells per cryotube in 1 ml of freezing medium (07930, STEMCELL Technologies) for further use in co-culture.

Fresh monocytes were seeded in 96-well flat-bottom treated culture plates (353072, Falcon) in culture medium (RPMI 1640 medium+1% Penicillin streptomycin+1% Sodium Pyruvate+1% L-Glutamine+10% Foetal Calf Serum) containing either, 50 ng/ml M-CSF (130-096-492, Miltenyi Biotec) for further M2-macrophage polarisation, or 50 ng/ml GM-CSF (130-093-866, Miltenyi Biotec) for further M1-macrophage polarisation. They were incubated at 37° C., 5% CO₂ for 6 days for differentiation into macrophages. Differentiated M0-macrophages were obtained at day 6.

50 ng/ml of IFN-γ (285-IF, R&D) was added to GM-CSF differentiated M0-macrophages for polarisation into pro-inflammatory M1-macrophages. 20 ng/ml of each of the following cytokines: IL-4 (130.093.922, Miltenyi Biotec), IL-10 (217-IL/CF, R&D) and TGF-β (130.095.066, Miltenyi Biotec) were added to M-CSF differentiated M0-macrophages for polarisation into immunosuppressive M2-macrophages. Differentiated M0-macrophages were incubated with cytokines at 37° C., 5% CO₂ for 2 days. M1 and M2 polarised macrophages were obtained at day 8. Polarised macrophages were activated with 100 ng/ml LPS (L4516, Sigma) for 4 hours at 37° C., 5% CO₂. Macrophages were then harvested and washed in culture medium. The binding of target antibodies on polarised M1- and M2-macrophages was assessed by flow cytometry following LPS activation.

M1- and M2-macrophages were seeded in classical flat-bottom 96-well plates at 20 000 cells/well in culture medium. They were incubated at 37° C., 5% CO₂ for 24 hours. CD4⁺ T cells were added to the macrophages at a ratio of 1 macrophage: 5 CD4 T cells. CD3/CD28 beads (111-32D, Gibco) were added to the co-culture to activate the CD4⁺ T cells at the ratio of 1 bead for 32 cells.

Test antibodies or corresponding isotypes were added to the co-culture at the final concentration of 10 μg/ml. Avelumab, an anti-PD-L1 monoclonal antibody, was used as a positive control. Macrophages and CD4⁺ T cells in co-culture were incubated at 37° C., 5% CO₂ for 5 days. Cell culture supernatants were harvested at day 14 and transferred into new V-bottom 96-well plates for cytokine analysis. The concentration of IFN-γ was measured. The quantification was performed using the Meso Scale Discovery technology according to the manufacturer's instructions (K151AEB-4, Meso Scale Discovery).

At least 5 donors were evaluated to take into account the heterogeneity between healthy donors. Each experimental condition was performed in triplicate and in one experiment.

The results of the assay are shown in Table 10.

TABLE 10
Reversion of M2-macrophage-mediated immunosuppression in response
to VSIG4 antibody treatment. Quantification of IFN-γ
secretion was used as a surrogate of T cell activation.

| | Donor showing an increase of IFN-γ release compared to isotype | | | | |
|---|---|---|---|---|---|
| | D344 | D345 | D348 | D349 | |
| Avelumab | ✓ | | ✓ | | 2D/4 |
| Atezolizumab | ✓ | | ✓ | ✓ | 3D/4 |
| SA2281 | ✓ | | ✓ | ✓ | 3D/4 |
| SA2297 | ✓ | | ✓ | ✓ | 3D/4 |
| SA3981 | | | | | 0D/4 |
| A2 | | | | | 0D/4 |

Both anti-VSIG4 antibodies SA2281 and SA2297 induce the release of IFN-γ by the CD4⁺ T cells, indicating that they trigger T cell activation. In contrast, SA3981, although binding to same VSIG4 region as SA2281, shows no immunosuppressive activity.

### 7.3 TAM Polarisation and T Cell Activation

General Design

A tripartite coculture assay is used to investigate the activity of the SA2281 and SA2297 antibodies against TAMs.

In a tripartite co-culture assay, macrophages are polarised by tumour cells into macrophages which are more akin to physiological macrophages, i.e., closer to in vivo TAM. This is obtained by using tumour cell lines environment and allowing cell-cell interaction. These TAM-like macrophages are capable of inhibiting T cell proliferation. In such an assay, the efficacy of the two monoclonal antibodies is assessed on TAM-like repolarisation to M1, T cell activation/reactivation and tumour cell killing.

PBMC from Healthy Donors:

Macrophages and CD3 T cells are obtained from healthy patient fresh blood. Briefly, peripheral mononuclear blood cells (PBMC) are isolated from healthy donors' cytapheresis ring using Ficoll gradient. PBMC ring is harvested and washed several times in PBS 2% FBS before proceeding a red blood cell lysis step. After new washes, a first CD14⁺ cells positive selection is achieved using Miltenyi Biotec autoMACS® Pro Separator and CD14 MicroBeads Human kit (130-050-201, Miltenyi Biotec), according to the manufacturer's instructions. Purified CD14⁺ cells are seeded in 100 mm thermosensitive UpCell Petri dishes (174902, ThermoFisher) and treated with 50 ng/ml M-CSF for 6 days (with refill with M-CSF containing medium at day 3) in order to generate M0-2 macrophages.

Negative fraction of autoMACS® Pro Separator CD14⁺ cells selection step is used for isolating CD3 T cells by negative selection using the EasySep™ Human T cell Enrichment Kit from StemCell Technologies (#19051, StemCell Technologies) according to the manufacturer's instructions. Isolated CD3 T cells are then frozen and thawed 24 h before their use for coculture.

Cell Lines:

MDA-MB-231 (triple negative breast cancer), NCI-H1975 (lung) and SKMEL-5 (melanoma) are obtained from ATCC, and are routinely grown in DMEM or RPM11640 media supplemented with 10% FBS. Those cells are demonstrated to induce macrophages polarization in TAM-like phenotype, and to induce VSIG4 expression on those TAM-like macrophages.

Tripartite Co-Culture Assay:

On Day 6, differentiated M0-2 macrophages are harvested and cocultured with tumour cell line at 1:1 ratio for 48 h in order to allow macrophage polarisation in TAM-like phenotype. After 2 days of bipartite coculture, CD3 T cells are activated using CD3/CD28 beads (11132D, Gibco) and added in bipartite coculture at 1:5 macrophage:CD3 T cells ratio, to generate tripartite coculture for 5 more days, with or without tested monoclonal antibodies or corresponding control isotype.

First readout is performed by flow cytometry on Day 8 to check TAM-like macrophage phenotype after polarisation step using cell surface markers CD80/CD86/CD163/CD200R/CD206/CD14. On Day 13, tumour cell killing and CD3 T lymphocyte activation and proliferation were assessed.

Tumour Cell Viability/Apoptosis:

The effect of anti-VSIG4 antibodies on tumour cells killing is assessed either by the evaluation of tumour cells viability using the CellTiter-Glo® luminescent cell viability assay (PROMEGA®) or by the evaluation of early markers of apoptosis such as 7AAD by flow cytometry.

The experimental scheme is shown in FIG. 16.

Preliminary results confirm the inhibition of VSIG4 anti-inflammatory and immunosuppressive functions demonstrated during in vitro assays. 7-4. Conclusion

The inflammatory and immunosuppressive assays show that both SA2281 and SA2297 promote M1 pro-inflammatory functions and antagonise M2 immunosuppressive activity, consistent with an effect on TAM polarisation towards an M1 phenotype. In contrast, SA3981, although sharing some of the functions of SA2281 and SA2297 (binding the M7 region of VSIG4, blocking the VSIG4/C3b and VSIG4/iC3b interactions), fails to affect the anti-inflammatory, pro-tumour activity of M2 macrophages. Thus SA2281 and SA2297 are both specifically capable of inducing an immune response, thereby conferring protective anti-tumour immunity.

1) A monoclonal anti-VSIG4 antibody, or an antigen-biding fragment thereof, selected in the group consisting of:
a) an antibody comprising the three heavy-chain CDRs of sequences SEQ ID Nos. 3, 4 and 5 and the three light-chain CDRs of sequences SEQ ID Nos. 6, 7 and 8; and
b) an antibody comprising the three heavy-chain CDRs of sequences SEQ ID Nos. 9, 10 and 11 and the three light-chain CDRs of sequences SEQ ID Nos. 12, 13 and 14.

2) The monoclonal anti-VSIG4 antibody, or antigen-biding fragment thereof, of claim 1, wherein the antibody is selected among single chain antibodies, camelised antibodies, chimeric antibodies, humanised antibodies, and human antibodies.

3) The monoclonal anti-VSIG4 antibody, or antigen-biding fragment thereof, of any one of claims 1 and 2, wherein the antibody is a human antibody.

4) The monoclonal anti-VSIG4 antibody, or antigen-biding fragment thereof, of any one of claims 1 to 3, wherein the antibody is selected among IgA1 antibodies, IgA2 antibodies, IgD antibodies, IgE antibodies, IgG1 antibodies, IgG2 antibodies, IgG3 antibodies, IgG4 antibodies and IgM antibodies.

5) The monoclonal anti-VSIG4 antibody, or antigen-biding fragment thereof, of any one of claims 1 to 3, wherein the antigen-biding fragment is selected in the group consisting of Fab, Fab′, (Fab′)₂, Fv, scFv (sc for single chain), Bis-scFv, scFv-Fc fragments, Fab2, Fab3, minibodies, diabodies, triabodies, tetrabodies, and nanobodies.

6) The monoclonal anti-VSIG4 antibody, or antigen-binding fragment thereof, of any one of claims 1 to 5, wherein said antigen-binding fragment is an scFv.

7) The monoclonal anti-VSIG4 antibody, or antigen-biding fragment thereof, of any one of claims 1 to 6, wherein the antibody is selected in the group consisting of:
a) an antibody comprising a heavy chain variable domain of sequence SEQ ID No. 45 or any sequence exhibiting at least 80%, 85%, 90%, 95% or 98% identity with SEQ ID No. 45 and the three light-chain CDRs of sequences SEQ ID Nos. 6, 7 and 8; and
b) an antibody comprising, or consisting of, a heavy chain variable domain of sequence SEQ ID No. 47 or any sequence exhibiting at least 80%, 85%, 90%, 95% or 98% identity with SEQ ID No. 47 and the three light-chain CDRs of sequences SEQ ID Nos. 12, 13 and 14.

8) The monoclonal anti-VSIG4 antibody, or antigen-binding fragment thereof, of any one of claims 1 to 6, the antibody being selected in the group consisting of:
a) an antibody comprising a light chain variable domain of sequence SEQ ID No. 46 or any sequence exhibiting at least 80%, 85%, 90%, 95% or 98% identity with SEQ ID No. 46 and the three heavy-chain CDRs of sequences SEQ ID Nos. 3, 4, and 5;
b) an antibody comprising a light chain variable domain of sequence SEQ ID No. 48 or any sequence exhibiting at least 80%, 85%, 90%, 95% or 98% identity with SEQ ID No. 48 and the three heavy-chain CDRs of sequences SEQ ID Nos. 9, 10, and 11.

9) The monoclonal anti-VSIG4 antibody, or antigen-binding fragment thereof, of any one of claims 1 to 8, the antibody being selected in the group consisting of:
a) an antibody comprising a heavy chain variable domain of sequence SEQ ID No. 45 or any sequence exhibiting at least 80%, 85%, 90%, 95% or 98% identity with SEQ ID No. 45 and a light chain variable domain of sequence SEQ ID No. 46 or any sequence exhibiting at least 80%, 85%, 90%, 95% or 98% identity with SEQ ID No. 46;
b) an antibody comprising a heavy chain variable domain of sequence SEQ ID No. 47 or any sequence exhibiting at least 80%, 85%, 90%, 95% or 98% identity with SEQ ID No. 47 and a light chain variable domain of sequence SEQ ID No. 48 or any sequence exhibiting at least 80%, 85%, 90%, 95% or 98% identity with SEQ ID NO. 48.

10) An immunoconjugate comprising the monoclonal anti-VSIG4 antibody, or antigen-binding fragment thereof, of any one of claims 1 to 9, wherein said antibody is conjugated to a cytotoxic agent.

11) A polynucleotide encoding a variable region of a light chain (V_{L}) for the monoclonal anti-VSIG4 antibody, or antigen-binding fragment thereof, of any one of claims 1 to 9.

12) A polynucleotide encoding a variable region of a heavy chain (V_{H}) for the monoclonal anti-VSIG4 antibody, or antigen-binding fragment thereof, of any one of claims 1 to 9.

13) A polynucleotide encoding a V_{L} for the monoclonal anti-VSIG4 antibody, or antigen-binding fragment thereof, of any one of claims 1 to 9 and a V_{H} for the monoclonal anti-VSIG4 antibody, or antigen-binding fragment thereof, of any one of claims 1 to 9.

14) An expression vector comprising:
a polynucleotide according to claim 11;
a polynucleotide according to claim 12;
a polynucleotide according to claim 11 and a polynucleotide according to claim 12; or
a polynucleotide according to claim 13.

15) A host cell transformed with the expression vector of claim 14.

16) A method of producing the monoclonal anti-VSIG4 antibody, or antigen-binding fragment thereof, of any one of claims 1 to 9 comprising:
a) culturing the host cell of claim 15 under suitable conditions; and
b) recovering the monoclonal anti-VSIG4 antibody, or antigen-binding fragment thereof, from the culture medium or from the cultured cells.

17) A pharmaceutical composition comprising the monoclonal anti-VSIG4 antibody, or antigen-binding fragment thereof, of any one of claims 1 to 9, or the immunoconjugate of claim 10, and a pharmaceutically acceptable carrier and/or an excipient.

18) The pharmaceutical composition of claim 17, further comprising an immune checkpoint inhibitor.

19) The pharmaceutical composition of claim 18, wherein said immune checkpoint inhibitor is an inhibitor of any one of CTLA-4, PDL1, PDL2, PD1, B7-H3, B7-H4, BTLA, HVEM, TIM3, GAL9, LAG3, VISTA, PSG-L1, TIGIT, KIR, 2B4, CD160, CGEN-15049, CHK1 and CHK2 kinases, IDO1, A2aR, and any of the various B-7 family ligands.

20) The pharmaceutical composition of any one of claims 17 and 18, wherein said immune checkpoint inhibitor is selected in the group consisting of ipilimumab, pembrolizumab, nivolumab, cemiplimab, pidilizumab, atezolizumab, avelumab, durvalumab, tiragolumab, vibostolimab, BMS 936559, JNJ 61610588, urelumab, 9B12, PF-04518600, BMS-986016, TSR-022, MBG453, MED16469, MED16383, and epacadostat.

21) The pharmaceutical composition of any one of claims 17 and 18, for simultaneous, separate, or sequential use.

22) The monoclonal anti-VSIG4 antibody, or antigen-binding fragment thereof, of any one of claims 1 to 9, or the immunoconjugate of claim 10, or the pharmaceutical composition of any one of claims 17 to 21, for use in the treatment of a cancer in a patient.

23) The monoclonal anti-VSIG4 antibody, or antigen-binding fragment thereof for the use of claim 22, wherein the use comprises inducing an immune response in the patient.

24) The monoclonal anti-VSIG4 antibody, or antigen-binding fragment thereof, of any one of claims 1 to 9, or the immunoconjugate of claim 10, or the pharmaceutical composition of any one of claims 17 to 21, for the use of claim 23, wherein the immune response includes polarisation of macrophages, notably TAM, to an M1 phenotype, induction of pro-inflammatory cytokines release by macrophages, induction of CD4⁺ T cell proliferation, induction of CD8⁺ T cell proliferation, induction of CD4⁺ T cell cytokine production, and induction of CD8⁺ T cell cytokine production.

25) The monoclonal anti-VSIG4 antibody, or antigen-binding fragment thereof, of any one of claims 1 to 9, or the immunoconjugate of claim 10, or the pharmaceutical composition of any one of claims 17 to 21, for the use of any one of claims 22 to 24, wherein the cancer is selected from bladder cancer, breast cancer, cervical cancer, colon cancer, endometrial cancer, oesophageal cancer, fallopian tube cancer, gall bladder cancer, gastrointestinal cancer, head-and-neck cancer, haematological cancer (e.g., leukaemia, lymphoma, or myeloma), laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma, and prostate cancer.

26) An in vitro method for detecting a VSIG4-expressing cancer in a subject, said method comprising the steps of:
a) contacting a biological sample of said subject with the monoclonal anti-VSIG4 antibody, or antigen-binding fragment thereof, of any one of claims 1 to 9; and
b) detecting the binding of said antibody, or antigen-binding fragment thereof, with said biological sample,
wherein the binding of the antiVSIG4 antibody indicates the presence of a VSIG4-expressing cancer.

27) The method of claim 26, wherein the monoclonal anti-VSIG4 antibody, or antigen-binding fragment thereof, is Labelled with a detectable label.


SEQUENCE LISTING (87 sequences)

SEQ ID NO: 1 - protein, 399 aa, Homo sapiens
MGILLGLLLLGHLTVDTYGRPILEVPESVTGPWKGDVNLPCTYDPLQGYTQVLVKWLVQRGSDPVTIFLRDSSGDHIQQAKYQGRLHVSHKVPGDVSLQLSTLEMDDRSHYTCEVTWQTPDGNQVVRDKITELRVQKLSVSKPTVTTGSGYGFTVPQGMRISLQCQARGSPPISYIWYKQQTNNQEPIKVATLSTLLFKPAVIADSGSYFCTAKGQVGSEQHSDIVKFVVKDSSKLLKTKTEAPTTMTYPLKATSTVKQSWDWTTDMDGYLGETSAGPGKSLPVFAIILIISLCCMVVFTMAYIMLCRKTSQQEHVYEAARAHAREANDSGETMRVAIFASGCSSDEPTSQNLGNNYSDEPCIGQEYQIIAQINGNYARLLDTVPLDYEFLATEGKSVC

SEQ ID NO: 2 - protein, 305 aa, Homo sapiens
MGILLGLLLLGHLTVDTYGRPILEVPESVTGPWKGDVNLPCTYDPLQGYTQVLVKWLVQRGSDPVTIFLRDSSGDHIQQAKYQGRLHVSHKVPGDVSLQLSTLEMDDRSHYTCEVTWQTPDGNQVVRDKITELRVQKHSSKLLKTKTEAPTTMTYPLKATSTVKQSWDWTTDMDGYLGETSAGPGKSLPVFAIILIISLCCMVVFTMAYIMLCRKTSQQEHVYEAARAHAREANDSGETMRVAIFASGCSSDEPTSQNLGNNYSDEPCIGQEYQIIAQINGNYARLLDTVPLDYEFLATEGKSVC

SEQ ID NO: 3 - protein, 8 aa, Homo sapiens
GFTFSTYA

SEQ ID NO: 4 - protein, 8 aa, Homo sapiens
VSHDGTYK

SEQ ID NO: 5 - protein, 11 aa, Homo sapiens
GRHAAGSSFDY

SEQ ID NO: 6 - protein, 6 aa, Homo sapiens
RDISNY

SEQ ID NO: 7 - protein, 3 aa, Homo sapiens
KTS

SEQ ID NO: 8 - protein, 9 aa, Homo sapiens
QQSYRTPYT

SEQ ID NO: 9 - protein, 8 aa, Homo sapiens
GFIFSDYY

SEQ ID NO: 10 - protein, 8 aa, Homo sapiens
ISPSGYTI

SEQ ID NO: 11 - protein, 14 aa, Homo sapiens
ARRFSSSWSGYFDY

SEQ ID NO: 12 - protein, 6 aa, Homo sapiens
QNINRW

SEQ ID NO: 13 - protein, 3 aa, Homo sapiens
KAF

SEQ ID NO: 14 - protein, 10 aa, Homo sapiens
QQYLSYSSSI

SEQ ID NO: 15 - protein, 8 aa, Homo sapiens
GFTFSDYY

SEQ ID NO: 16 - protein, 8 aa, Homo sapiens
ISSSGSTI

SEQ ID NO: 17 - protein, 10 aa, Homo sapiens
ARGYYYGMDV

SEQ ID NO: 18 - protein, 9 aa, Homo sapiens
SSDVGDYNL

SEQ ID NO: 19 - protein, 3 aa, Homo sapiens
EVS

SEQ ID NO: 20 - protein, 10 aa, Homo sapiens
SSYKTGDTWL

SEQ ID NO: 21 - protein, 25 aa, Homo sapiens
QVQLVESGGGVVQPGRSLRVSCAAS

SEQ ID NO: 22 - protein, 17 aa, Homo sapiens
MHWVRQAPGKGLEWLAV

SEQ ID NO: 23 - protein, 38 aa, Homo sapiens
YYGDSVKGRFSISRDNSKNTLYLQMNSLRSEDTAVYYC

SEQ ID NO: 24 - protein, 11 aa, Homo sapiens
WGQGTVVTVSS

SEQ ID NO: 25 - protein, 26 aa, Homo sapiens
DIQMTQSPSSLSASVGDRVTITCQAS

SEQ ID NO: 26 - protein, 17 aa, Homo sapiens
LNWYQQKPGKAPKLLIY

SEQ ID NO: 27 - protein, 36 aa, Homo sapiens
NLQRGVPSRFSGSGSGTEFTLTISRLQPDDSATYYC

SEQ ID NO: 28 - protein, 10 aa, Homo sapiens
FGQGTKLEIK

SEQ ID NO: 29 - protein, 25 aa, Homo sapiens
QVQLVESGGGVVQPGGSLRLSCAAS

SEQ ID NO: 30 - protein, 17 aa, Homo sapiens
MSWVRQAPGKGLEWVSY

SEQ ID NO: 31 - protein, 38 aa, Homo sapiens
SYADSVKGRFTISRDNSKNSLYLQMNSLRAEDTAVYYC

SEQ ID NO: 32 - protein, 11 aa, Homo sapiens
WGHGALVTVSS

SEQ ID NO: 33 - protein, 26 aa, Homo sapiens
DIQMTQSPSTLSASVGDRVTITCRAS

SEQ ID NO: 34 - protein, 17 aa, Homo sapiens
LAWYQQKPGKAPKLLIY

SEQ ID NO: 35 - protein, 36 aa, Homo sapiens
RLHSGVPSRFSGSGSGTEFTLTITSLQPDDFATYYC

SEQ ID NO: 36 - protein, 10 aa, Homo sapiens
FGQGTKVEIK

SEQ ID NO: 37 - protein, 25 aa, Homo sapiens
QVQLVESGGGLVKPGGSLRLSCAAS

SEQ ID NO: 38 - protein, 17 aa, Homo sapiens
MSWIRQAPGKGLEWVSY

SEQ ID NO: 39 - protein, 38 aa, Homo sapiens
YYADSVKGRFTISRDNAKNSLYLQMNSLRAEDTAVYYC

SEQ ID NO: 40 - protein, 11 aa, Homo sapiens
WGQGTTVTVSS

SEQ ID NO: 41 - protein, 25 aa, Homo sapiens
QSALTQPASVSGSPGQSITISCTGT

SEQ ID NO: 42 - protein, 17 aa, Homo sapiens
VSWYQQYPGKAPKLILY

SEQ ID NO: 43 - protein, 36 aa, Homo sapiens
KRPSGVSNRFSGSKSGNTASLTISGLQAEDEADYYC

SEQ ID NO: 44 - protein, 10 aa, Homo sapiens
FGGGTKLTVL

SEQ ID NO: 45 - protein, 118 aa, Homo sapiens
QVQLVESGGGVVQPGRSLRVSCAASGFTFSTYAMHWVRQAPGKGLEWLAVVSHDGTYKYYGDSVKGRFSISRDNSKNTLYLQMNSLRSEDTAVYYCGRHAAGSSFDYWGQGTVVTVSS

SEQ ID NO: 46 - protein, 107 aa, Homo sapiens
DIQMTQSPSSLSASVGDRVTITCQASRDISNYLNWYQQKPGKAPKLLIYKTSNLQRGVPSRFSGSGSGTEFTLTISRLQPDDSATYYCQQSYRTPYTFGQGTKLEIK

SEQ ID NO: 47 - protein, 121 aa, Homo sapiens
QVQLVESGGGVVQPGGSLRLSCAASGFIFSDYYMSWVRQAPGKGLEWVSYISPSGYTISYADSVKGRFTISRDNSKNSLYLQMNSLRAEDTAVYYCARRFSSSWSGYFDYWGHGALVTVSS

SEQ ID NO: 48 - protein, 108 aa, Homo sapiens
DIQMTQSPSTLSASVGDRVTITCRASQNINRWLAWYQQKPGKAPKLLIYKAFRLHSGVPSRFSGSGSGTEFTLTITSLQPDDFATYYCQQYLSYSSSIFGQGTKVEIK

SEQ ID NO: 49 - protein, 117 aa, Homo sapiens
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGYYYGMDVWGQGTTVTVSS

SEQ ID NO: 50 - protein, 110 aa, Homo sapiens
QSALTQPASVSGSPGQSITISCTGTSSDVGDYNLVSWYQQYPGKAPKLILYEVSKRPSGVSNRFSGSKSGNTASLTISGLQAEDEADYYCSSYKTGDTWLFGGGTKLTVL

SEQ ID NO: 51 - DNA, 22 bp, Artificial
  Primer
cgtcccatcctggaagtgccag

SEQ ID NO: 52 - DNA, 23 bp, Artificial
  Primer
gctctttcctggcccagcactgg

SEQ ID NO: 53 - DNA, 792 bp, Artificial
  DNA (hVSIG4)
cgtcccatcctggaagtgccagagagtgtaacaggaccttggaaaggggatgtgaatcttccctgcacctatgaccccctgcaaggctacacccaagtcttggtgaagtggctggtacaacgtggctcagaccctgtcaccatctttctacgtgactcttctggagaccatatccagcaggcaaagtaccagggccgcctgcatgtgagccacaaggttccaggagatgtatccctccaattgagcaccctggagatggatgaccggagccactacacgtgtgaagtcacctggcagactcctgatggcaaccaagtcgtgagagataagattactgagctccgtgtccagaaactctctgtctccaagcccacagtgacaactggcagcggttatggcttcacggtgccccagggaatgaggattagccttcaatgccaggctcggggttctcctcccatcagttatatttggtataagcaacagactaataaccaggaacccatcaaagtagcaaccctaagtaccttactcttcaagcctgcggtgatagccgactcaggctcctatttctgcactgccaagggccaggttggctctgagcagcacagcgacattgtgaagtttgtggtcaaagactcctcaaagctactcaagaccaagactgaggcacctacaaccatgacataccccttgaaagcaacatctacagtgaagcagtcctgggactggaccactgacatggatggctaccttggagagaccagtgctgggccaggaaagagcctgcct

SEQ ID NO: 54 - protein, 264 aa, Artificial
  Protein (hVSIG4)
RPILEVPESVTGPWKGDVNLPCTYDPLQGYTQVLVKWLVQRGSDPVTIFLRDSSGDHIQQAKYQGRLHVSHKVPGDVSLQLSTLEMDDRSHYTCEVTWQTPDGNQVVRDKITELRVQKLSVSKPTVTTGSGYGFTVPQGMRISLQCQARGSPPISYIWYKQQTNNQEPIKVATLSTLLFKPAVIADSGSYFCTAKGQVGSEQHSDIVKFVVKDSSKLLKTKTEAPTTMTYPLKATSTVKQSWDWTTDMDGYLGETSAGPGKSLP

SEQ ID NO: 55 - protein, 517 aa, Artificial
  Fusion protein + leader sequence (LS-hVSIG4-Fc)
MGWSYIILFLVATAADVHSRPILEVPESVTGPWKGDVNLPCTYDPLQGYTQVLVKWLVQRGSDPVTIFLRDSSGDHIQQAKYQGRLHVSHKVPGDVSLQLSTLEMDDRSHYTCEVTWQTPDGNQVVRDKITELRVQKLSVSKPTVTTGSGYGFTVPQGMRISLQCQARGSPPISYIWYKQQTNNQEPIKVATLSTLLFKPAVIADSGSYFCTAKGQVGSEQHSDIVKFVVKDSSKLLKTKTEAPTTMTYPLKATSTVKQSWDWTTDMDGYLGETSAGPGKSLPASEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - DNA, 318 bp, Artificial
  DNA (hVSIG4-V)
cgtcccatcctggaagtgccagagagtgtaacaggaccttggaaaggggatgtgaatcttccctgcacctatgaccccctgcaaggctacacccaagtcttggtgaagtggctggtacaacgtggctcagaccctgtcaccatctttctacgtgactcttctggagaccatatccagcaggcaaagtaccagggccgcctgcatgtgagccacaaggttccaggagatgtatccctccaattgagcaccctggagatggatgaccggagccactacacgtgtgaagtcacctggcagactcctgatggcaaccaagtc

SEQ ID NO: 57 - protein, 106 aa, Artificial
  Protein (hVSIG4-V)
RPILEVPESVTGPWKGDVNLPCTYDPLQGYTQVLVKWLVQRGSDPVTIFLRDSSGDHIQQAKYQGRLHVSHKVPGDVSLQLSTLEMDDRSHYTCEVTWQTPDGNQV

SEQ ID NO: 58 - protein, 359 aa, Artificial
  Fusion protein + leader sequence (LS-hVSIG4-V-Fc)
MGWSYIILFLVATAADVHSRPILEVPESVTGPWKGDVNLPCTYDPLQGYTQVLVKWLVQRGSDPVTIFLRDSSGDHIQQAKYQGRLHVSHKVPGDVSLQLSTLEMDDRSHYTCEVTWQTPDGNQVASEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - DNA, 348 bp, Artificial
  DNA (VSIG4-M1)
cgtcccatcctgaccgccccccacagcctggccggaccttggaaaggggatgtgaatcttccctgcacctatgaccccctgcaaggctacacccaagtcttggtgaagtggctggtacaacgtggctcagaccctgtcaccatctttctacgtgactcttctggagaccatatccagcaggcaaagtaccagggccgcctgcatgtgagccacaaggttccaggagatgtatccctccaattgagcaccctggagatggatgaccggagccactacacgtgtgaagtcacctggcagactcctgatggcaaccaagtcgtgagagataagattactgagctccgtgtt

SEQ ID NO: 60 - protein, 116 aa, Artificial
  Protein (VSIG4-M1)
RPILTAPHSLAGPWKGDVNLPCTYDPLQGYTQVLVKWLVQRGSDPVTIFLRDSSGDHIQQAKYQGRLHVSHKVPGDVSLQLSTLEMDDRSHYTCEVTWQTPDGNQVVRDKITELRV

SEQ ID NO: 61 - protein, 380 aa, Artificial
  Fusion protein + leader sequence (LS-VSIG4-M1-Fc)
MGWSYIILFLVATATDVHSQGAVGARPILTAPHSLAGPWKGDVNLPCTYDPLQGYTQVLVKWLVQRGSDPVTIFLRDSSGDHIQQAKYQGRLHVSHKVPGDVSLQLSTLEMDDRSHYTCEVTWQTPDGNQVVRDKITELRVLAASAASEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - DNA, 345 bp, Artificial
  DNA (VSIG4-M2)
cccatcctggaagtgccagagagtgtaacaggaccttggaaagggaccgtgcggatgccctgcagctatgaccccctgcaaggctacacccaagtcttggtgaagtggctggtacaacgtggctcagaccctgtcaccatctttctacgtgactcttctggagaccatatccagcaggcaaagtaccagggccgcctgcatgtgagccacaaggttccaggagatgtatccctccaattgagcaccctggagatggatgaccggagccactacacgtgtgaagtcacctggcagactcctgatggcaaccaagtcgtgagagataagattactgagctccgtgtt

SEQ ID NO: 63 - protein, 115 aa, Artificial
  Protein (VSIG4-M2)
PILEVPESVTGPWKGTVRMPCSYDPLQGYTQVLVKWLVQRGSDPVTIFLRDSSGDHIQQAKYQGRLHVSHKVPGDVSLQLSTLEMDDRSHYTCEVTWQTPDGNQVVRDKITELRV

SEQ ID NO: 64 - protein, 379 aa, Artificial
  Fusion protein + leader sequence (LS-VSIG4-M2-Fc)
MGWSYIILFLVATATDVHSQGAVGAPILEVPESVTGPWKGTVRMPCSYDPLQGYTQVLVKWLVQRGSDPVTIFLRDSSGDHIQQAKYQGRLHVSHKVPGDVSLQLSTLEMDDRSHYTCEVTWQTPDGNQVVRDKITELRVLAASAASEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - DNA, 345 bp, Artificial
  DNA (VSIG4-M3)
cccatcctggaagtgccagagagtgtaacaggaccttggaaaggggatgtgaatcttccctgcacctatgaccccctgcaaggctacacccaagtcttggtgaagtggctggtactgcggaactaccaccccgccaccatctttctacgtgactcttctggagaccatatccagcaggcaaagtaccagggccgcctgcatgtgagccacaaggttccaggagatgtatccctccaattgagcaccctggagatggatgaccggagccactacacgtgtgaagtcacctggcagactcctgatggcaaccaagtcgtgagagataagattactgagctccgtgtc

SEQ ID NO: 66 - protein, 115 aa, Artificial
  Protein (VSIG4-M3)
PILEVPESVTGPWKGDVNLPCTYDPLQGYTQVLVKWLVLRNYHPATIFLRDSSGDHIQQAKYQGRLHVSHKVPGDVSLQLSTLEMDDRSHYTCEVTWQTPDGNQVVRDKITELRV

SEQ ID NO: 67 - protein, 379 aa, Artificial
  Fusion protein + leader sequence (LS-VSIG4-M3-Fc)
MGWSYIILFLVATATDVHSQGAVGAPILEVPESVTGPWKGDVNLPCTYDPLQGYTQVLVKWLVLRNYHPATIFLRDSSGDHIQQAKYQGRLHVSHKVPGDVSLQLSTLEMDDRSHYTCEVTWQTPDGNQVVRDKITELRVLAASAASEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - DNA, 345 bp, Artificial
  DNA (VSIG4-M4)
cccatcctggaagtgccagagagtgtaacaggaccttggaaaggggatgtgaatcttccctgcacctatgaccccctgcaaggctacacccaagtcttggtgaagtggctggtacaacgtggctcagaccctgtcaccatctttctacgtgactcttctggagaccatgtgcagcagaccaagttccggggccgcctgcatgtgagccacaaggttccaggagatgtatccctccaattgagcaccctggagatggatgaccggagccactacacgtgtgaagtcacctggcagactcctgatggcaaccaagtcgtgagagataagattactgagctccgtgtt

SEQ ID NO: 69 - protein, 115 aa, Artificial
  Protein (VSIG4-M4)
PILEVPESVTGPWKGDVNLPCTYDPLQGYTQVLVKWLVQRGSDPVTIFLRDSSGDHVQQTKFRGRLHVSHKVPGDVSLQLSTLEMDDRSHYTCEVTWQTPDGNQVVRDKITELRV

SEQ ID NO: 70 - protein, 379 aa, Artificial
  Fusion protein + leader sequence (LS-VSIG4-M4-Fc)
MGWSYIILFLVATATDVHSQGAVGAPILEVPESVTGPWKGDVNLPCTYDPLQGYTQVLVKWLVQRGSDPVTIFLRDSSGDHVQQTKFRGRLHVSHKVPGDVSLQLSTLEMDDRSHYTCEVTWQTPDGNQVVRDKITELRVLAASAASEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - DNA, 345 bp, Artificial
  DNA (VSIG4-M5)
cccatcctggaagtgccagagagtgtaacaggaccttggaaaggggatgtgaatcttccctgcacctatgaccccctgcaaggctacacccaagtcttggtgaagtggctggtacaacgtggctcagaccctgtcaccatctttctacgtgactcttctggagaccatatccagcaggcaaagtaccagggccgcctggaggtgagccggcagcccccaggagatgtatccctccaattgagcaccctggagatggatgaccggagccactacacgtgtgaagtcacctggcagactcctgatggcaaccaagtcgtgagagataagattactgagctccgtgtt

SEQ ID NO: 72 - protein, 115 aa, Artificial
  Protein (VSIG4-M5)
PILEVPESVTGPWKGDVNLPCTYDPLQGYTQVLVKWLVQRGSDPVTIFLRDSSGDHIQQAKYQGRLEVSRQPPGDVSLQLSTLEMDDRSHYTCEVTWQTPDGNQVVRDKITELRV

SEQ ID NO: 73 - protein, 379 aa, Artificial
  Fusion protein + leader sequence (LS-VSIG4-M5-Fc)
MGWSYIILFLVATATDVHSQGAVGAPILEVPESVTGPWKGDVNLPCTYDPLQGYTQVLVKWLVQRGSDPVTIFLRDSSGDHIQQAKYQGRLEVSRQPPGDVSLQLSTLEMDDRSHYTCEVTWQTPDGNQVVRDKITELRVLAASAASEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - DNA, 345 bp, Artificial
  DNA (VSIG4-M6)
cccatcctggaagtgccagagagtgtaacaggaccttggaaaggggatgtgaatcttccctgcacctatgaccccctgcaaggctacacccaagtcttggtgaagtggctggtacaacgtggctcagaccctgtcaccatctttctacgtgactcttctggagaccatatccagcaggcaaagtaccagggccgcctgcatgtgagccacaaggttccaggagatgtagccctgaccctgaaccccctggagatggatgaccggagccactacacgtgtgaagtcacctggcagactcctgatggcaaccaagtcgtgagagataagattactgagctccgtgtt

SEQ ID NO: 75 - protein, 115 aa, Artificial
  Protein (VSIG4-M6)
PILEVPESVTGPWKGDVNLPCTYDPLQGYTQVLVKWLVQRGSDPVTIFLRDSSGDHIQQAKYQGRLHVSHKVPGDVALTLNPLEMDDRSHYTCEVTWQTPDGNQVVRDKITELRV

SEQ ID NO: 76 - protein, 379 aa, Artificial
  Fusion protein + leader sequence (LS-VSIG4-M6-Fc)
MGWSYIILFLVATATDVHSQGAVGAPILEVPESVTGPWKGDVNLPCTYDPLQGYTQVLVKWLVQRGSDPVTIFLRDSSGDHIQQAKYQGRLHVSHKVPGDVALTLNPLEMDDRSHYTCEVTWQTPDGNQVVRDKITELRVLAASAASEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - DNA, 345 bp, Artificial
  DNA (VSIG4-M7)
cccatcctggaagtgccagagagtgtaacaggaccttggaaaggggatgtgaatcttccctgcacctatgaccccctgcaaggctacacccaagtcttggtgaagtggctggtacaacgtggctcagaccctgtcaccatctttctacgtgactcttctggagaccatatccagcaggcaaagtaccagggccgcctgcatgtgagccacaaggttccaggagatgtatccctccaattgagcaccctggagatggatgaccagggctactacgtgtgcgccgtcacctggcagactcctgatggcaaccaagtcgtgagagataagattactgagctccgtgtt

SEQ ID NO: 78 - protein, 115 aa, Artificial
  Protein (VSIG4-M7)
PILEVPESVTGPWKGDVNLPCTYDPLQGYTQVLVKWLVQRGSDPVTIFLRDSSGDHIQQAKYQGRLHVSHKVPGDVSLQLSTLEMDDQGYYVCAVTWQTPDGNQVVRDKITELRV

SEQ ID NO: 79 - protein, 379 aa, Artificial
  Fusion protein + leader sequence (LS-VSIG4-M7-Fc)
MGWSYIILFLVATATDVHSQGAVGAPILEVPESVTGPWKGDVNLPCTYDPLQGYTQVLVKWLVQRGSDPVTIFLRDSSGDHIQQAKYQGRLHVSHKVPGDVSLQLSTLEMDDQGYYVCAVTWQTPDGNQVVRDKITELRVLAASAASEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - DNA, 345 bp, Artificial
  DNA (VSIG4-M8)
cccatcctggaagtgccagagagtgtaacaggaccttggaaaggggatgtgaatcttccctgcacctatgaccccctgcaaggctacacccaagtcttggtgaagtggctggtacaacgtggctcagaccctgtcaccatctttctacgtgactcttctggagaccatatccagcaggcaaagtaccagggccgcctgcatgtgagccacaaggttccaggagatgtatccctccaattgagcaccctggagatggatgaccggagccactacacgtgtgaagtcacctggcaggaccaggccggccacctgatcgtgagagataagattactgagctccgtgtt

SEQ ID NO: 81 - protein, 115 aa, Artificial
  Protein (VSIG4-M8)
PILEVPESVTGPWKGDVNLPCTYDPLQGYTQVLVKWLVQRGSDPVTIFLRDSSGDHIQQAKYQGRLHVSHKVPGDVSLQLSTLEMDDRSHYTCEVTWQDQAGHLIVRDKITELRV

SEQ ID NO: 82 - protein, 379 aa, Artificial
  Fusion protein + leader sequence (LS-VSIG4-M8-Fc)
MGWSYIILFLVATATDVHSQGAVGAPILEVPESVTGPWKGDVNLPCTYDPLQGYTQVLVKWLVQRGSDPVTIFLRDSSGDHIQQAKYQGRLHVSHKVPGDVSLQLSTLEMDDRSHYTCEVTWQDQAGHLIVRDKITELRVLAASAASEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 513 aa, Artificial
  Fusion Protein (signal peptide-hVSIG4(L)-Fc)
  MISC_FEATURE: human IgG1 Fc domain, including hinge
QGILLGLLLLGHLTVDTYGRPILEVPESVTGPWKGDVNLPCTYDPLQGYTQVLVKWLVQRGSDPVTIFLRDSSGDHIQQAKYQGRLHVSHKVPGDVSLQLSTLEMDDRSHYTCEVTWQTPDGNQVVRDKITELRVQKLSVSKPTVTTGSGYGFTVPQGMRISLQCQARGSPPISYIWYKQQTNNQEPIKVATLSTLLFKPAVIADSGSYFCTAKGQVGSEQHSDIVKFVVKDSSKLLKTKTEAPTTMTYPLKATSTVKQSWDWTTDMDGYLGETSAGPGKSLPAAADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 419 aa, Artificial
  Fusion Protein (signal peptide-hVSIG4(S)-Fc)
MGILLGLLLLGHLTVDTYGRPILEVPESVTGPWKGDVNLPCTYDPLQGYTQVLVKWLVQRGSDPVTIFLRDSSGDHIQQAKYQGRLHVSHKVPGDVSLQLSTLEMDDRSHYTCEVTWQTPDGNQVVRDKITELRVQKHSSKLLKTKTEAPTTMTYPLKATSTVKQSWDWTTDMDGYLGETSAGPGKSLPAAADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 6 aa, Artificial
  Linker
QGAVGA

SEQ ID NO: 87 - protein, 7 aa, Artificial
  Linker
LAASAAS